(19) United States
(12) Patent Application Publication
(10) Pub. No.: US 2020/0009088 A1
(43) Pub. Date: Jan. 9, 2020

(54) COMPOSITIONS AND METHODS FOR TREATING ANDROGEN-INDEPENDENT CANCER

(71) Applicant: The Regents of the University of California, Oakland, CA (US)

(72) Inventors: Allen Gao, Davis, CA (US); Pui-Kai Li, Columbus, OH (US)

(73) Assignees: The Regents of the University of California, Oakland, CA (US); Ohio State Innovation Foundation, Columbus, OH (US); U.S. Government represented by the Department of Veterans Affairs, Washington, DC (US)

(21) Appl. No.: 16/510,621

(22) Filed: Jul. 12, 2019

Related U.S. Application Data
(63) Continuation of application No. PCT/US2018/014261, filed on Jan. 18, 2018.
(60) Provisional application No. 62/448,094, filed on Jan. 19, 2017.

(51) Int. Cl. A61K 31/167; A61P 35/00; A61K 31/275; A61K 31/4439; A61K 31/58

(52) U.S. Cl. CPC A61K 31/167; A61P 35/00; A61K 31/58; A61K 31/4439; A61K 31/275

(57) ABSTRACT

Compositions and methods are provided herein for the prevention and treatment of cancer, including prostate cancer, breast cancer, and androgen-independent cancer. In one aspect, the present invention provides a composition comprising a niclosamide analog and an antiandrogen drug. In some embodiments, the compositions and methods provided herein inhibit a mutant or splice variant androgen receptor. In particular embodiments, the compositions and methods provided herein reduce the androgen independence of a cancer. Kits are also provided herein.

### CROSS-REFERENCE TO RELATED APPLICATIONS

This application is a continuation of International Patent Application No. PCT/US2018/014261, filed Jan. 18, 2018, which claims priority to U.S. Provisional Application No. 62/448,094, filed Jan. 19, 2017, the disclosures of which are herein incorporated by reference in their entirety for all purposes.

### REFERENCE TO A “SEQUENCE LISTING”

The Sequence Listing written in file SequenceListing 070772-224210US-1147683.txt created on Jul. 12, 2019, 14,022 bytes, machine format IBM-PC, MS-Windows operating system, is hereby incorporated by reference in its entirety for all purposes.

### BACKGROUND OF THE INVENTION

Prostate cancer is the second leading cause of cancer-related death and the most commonly diagnosed cancer in men, with an estimated 220,800 new cases annually in the United States alone. First-line treatments for prostate cancer aim to reduce circulating androgen levels through the use of androgen deprivation therapies (ADT). While ADT is initially effective at reducing prostate cancer growth, after two to three years of treatment the majority of patients progress to castration-resistant prostate cancer (CRPC) and tumor growth will proceed even in the presence of castrate levels of androgen. At this point of disease progression, the number of therapeutic options becomes very limited.

Treatment of CRPC currently includes either the administration of taxanes, such as docetaxel and cabazitaxel, which interrupt the growth of fast-dividing cells through disruption of microtubule function, or next-generation antiandrogen drugs such as enzalutamide and abiraterone. Unfortunately, it is estimated that about one-third of patients given abiraterone and about one-fourth of patients given enzalutamide fail to respond to initial treatment with these drugs. Furthermore, within 12-24 months of initiating treatment, even those who initially respond to the drugs often develop resistance. Accordingly, there is a need in the art for more effective treatments for androgen-independent cancers such as CRPC. The present invention satisfies this need, and provides related advantages as well.

### BRIEF SUMMARY OF THE INVENTION

In one aspect, the invention provides a composition comprising an antiandrogen drug and a compound according to Formula (I):

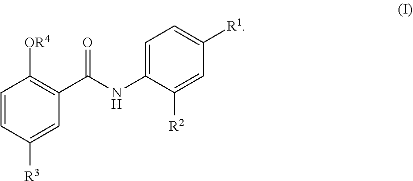

In some embodiments, R¹ is selected from the group consisting of X, CX₃, NO₂, OH, and alkoxy; R² is selected from the group consisting of H, X, CX₃, NO₂, OH, and alkoxy; R³ is selected from the group consisting of X, CX₃, NO₂, OH, and alkoxy; and R⁴ is selected from the group consisting of H and C(O)R⁵, wherein R⁵ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R¹ is CX₃ or NO₂. In other instances, R² is H or X. In some other instances, R³ is X. In other instances, R⁵ is C₂ alkyl or C₂ alkenyl. In particular instances, X is independently selected from the group consisting of F and Cl. In particular embodiments, the composistion further comprises a pharmaceutically acceptable carrier.

In some embodiments, the compound of Formula (I) is selected from the group consisting of

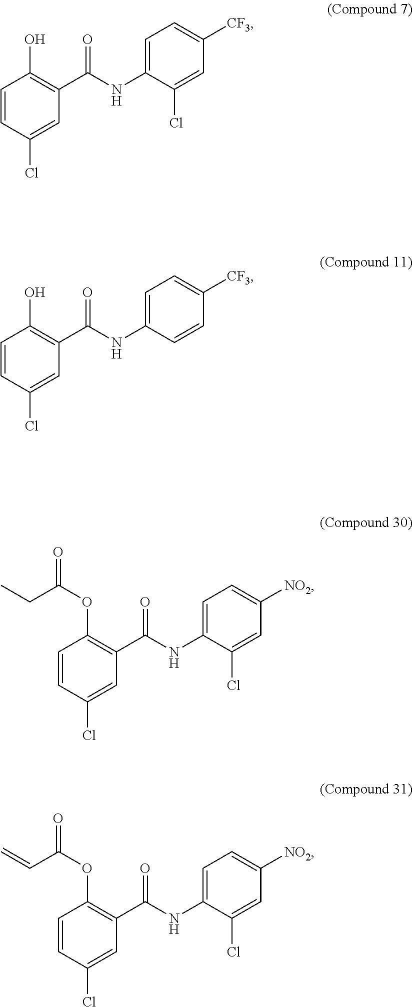 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.

In other embodiments, the antiandrogen drug is selected from the group consisting of a non-steroidal androgen receptor antagonist, a CYP17A1 inhibitor, and a combination thereof. In some instances, the antiandrogen drug is selected from the group consisting of bicalutamide, apalutamide, enzalutamide, abiraterone acetate, and a combination thereof.

In some embodiments, the composition inhibits the expression and/or activity of an androgen receptor or a variant thereof. In particular embodiments, the androgen receptor variant is selected from the group consisting of a splice variant, a mutant variant, and a combination thereof. In some instances, the splice variant is an AR-V1, AR-V3, AR-V7, AR-V9, and/or AR-V12 splice variant. In particular instances, the splice variant is an AR-V7 splice variant. In other instances, the mutant variant comprises one or more mutations selected from the group consisting of K581R, L702H, T878A, V716M, and a combination thereof relative to the amino acid sequence set forth in SEQ ID NO: 1.

In other embodiments, the composition is an effective inhibitor of cancer cell proliferation. In some instances, the cancer cell is a prostate cancer cell or a breast cancer cell. In other instances, the cancer cell is selected from the group consisting of an androgen-independent cancer cell, a metastatic cancer cell, a castrate-resistant cancer cell, a castration recurrent cancer cell, a hormone-resistant cancer cell, a metastatic castrate-resistant cancer cell, and a combination thereof.

In a second aspect, the invention provides a method for preventing or treating cancer in a subject. In some embodiments, the method comprises administering to the subject a therapeutically effective amount of a composition comprising an antiandrogen drug and a compound according to Formula (I):

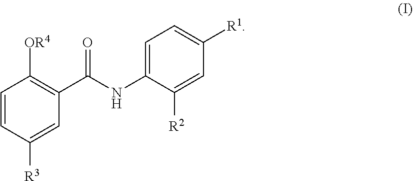

In particular embodiments, R¹ is selected from the group consisting of X, CX₃, NO₂, OH, and alkoxy; R² is selected from the group consisting of H, X, CX₃, NO₂, OH, and alkoxy; R³ is selected from the group consisting of X, CX₃, NO₂, OH, and alkoxy; and R⁴ is selected from the group consisting of H and C(O)R⁵, wherein R⁵ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R¹ is CX₃ or NO₂. In other instances, R² is H or X. In some other instances, R³ is X. In other instances, R⁵ is C₂ alkyl or C₂ alkenyl. In particular instances, X is independently selected from the group consisting of F and Cl. In particular embodiments, the composistion further comprises a pharmaceutically accpetable carrier.

In some embodiments, the compound of Formula (I) is selected from the group consisting of

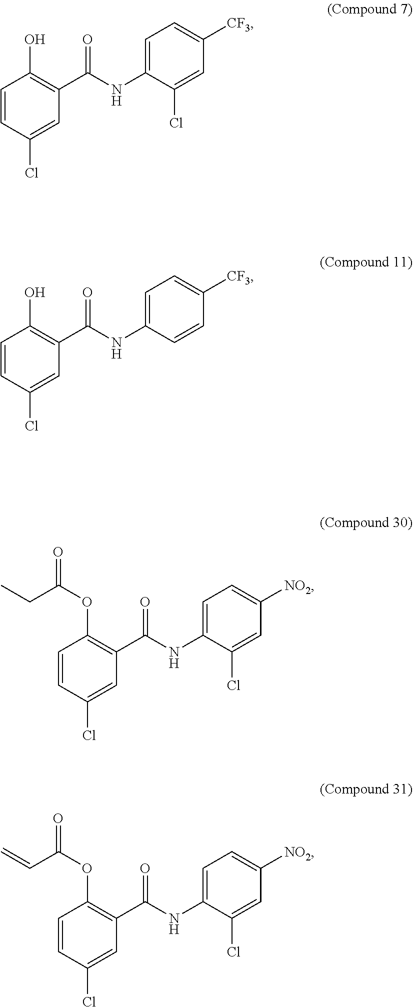 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.

In some embodiments, the antiandrogen drug is selected from the group consisting of a non-steroidal androgen receptor antagonist, a CYP17A1 inhibitor, and a combination thereof. In some instances, the antiandrogen drug is selected from the group consisting of bicalutamide, apalutamide, enzalutamide, abiraterone acetate, and a combination thereof.

In some embodiments, the expression and/or activity of an androgen receptor or a variant thereof is inhibited. In particular embodiments, the androgen receptor variant is selected from the group consisting of a splice variant, a mutant variant, and a combination thereof. In some instances, the splice variant is an AR-V1, AR-V3, AR-V7, AR-V9, and/or AR-V12 splice variant. In particular instances, the splice variant is an AR-V7 splice variant. In other instances, the mutant variant comprises one or more mutations selected from the group consisting of K581R, L702H, T878A, V716M, and a combination thereof relative to the amino acid sequence set forth in SEQ ID NO: 1.

In some embodiments, the cancer is prostate cancer or breast cancer. In other embodiments, the cancer is selected from the group consisting of an androgen-independent cancer, a metastatic cancer, a castrate-resistant cancer, a castration recurrent cancer, a hormone-resistant cancer, a metastatic castrate-resistant cancer, and a combination thereof. In some instances, the androgen independence, castrate resistance, or hormone resistance of the cancer is decreased or reversed.

In other embodiments, the antiandrogen drug and the compound of Formula (I) are given concomitantly. In some other embodiments, the antiandrogen drug and the compound of Formula (I) are given sequentially. In particular embodiments, the subject does not have cancer. In some embodiments, treating the subject results in an improvement in one or more symptoms of the cancer.

In some embodiments, a test sample is obtained from the subject before and/or after the antiandrogen drug and the compound of Formula (I) are administered to the subject. In some instances, the test sample comprises tissue, blood, or a combination thereof. In particular instances, the test tissue sample comprises cancer tissue. In some embodiments, the level of one or more biomarkers is determined in the sample. In some instances, the one or more biomarkers comprises prostate-specific antigen (PSA).

In some embodiments, the level of the one or more biomarkers in the test sample is compared to the level of the one or more biomarkers in a reference sample. In some instances, the reference sample is normal blood or tissue obtained from the same subject before and/or after the antiandrogen drug and the compound of Formula (I) are administered to the subject. In other instances, the reference sample is obtained from a different subject or a population of subjects. In some embodiments, the level of PSA in the test sample is higher than the level of PSA in the reference sample, and the test sample is obtained before the antiandrogen drug and the compound of Formula (I) are administered to the subject. In other embodiments, administering the antiandrogen drug and the compound of Formula (I) to the subject results in a decrease in the level of PSA in a test sample obtained from the subject after administration compared to a test sample obtained from the subject before administration.

In a third aspect, the invention provides a method for inhibiting the expression and/or activity of an androgen receptor in a cell. In some embodiments, the method comprises contacting the androgen receptor or the cell with a therapeutically effective amount of a composition comprising an antiandrogen drug and a compound according to Formula (I):

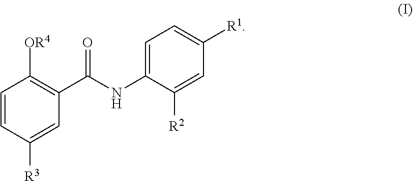

In particular embodiments, R¹ is selected from the group consisting of X, CX₃, NO₂, OH, and alkoxy; R² is selected from the group consisting of H, X, CX₃, NO₂, OH, and alkoxy; R³ is selected from the group consisting of X, CX₃, NO₂, OH, and alkoxy; and R⁴ is selected from the group consisting of H and C(O)R⁵, wherein R⁵ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R¹ is CX₃ or NO₂. In other instances, R² is H or X. In some other instances, R³ is X. In other instances, R⁵ is C₂ alkyl or C₂ alkenyl. In particular instances, X is independently selected from the group consisting of F and Cl. In particular embodiments, the composistion further comprises a pharmaceutically accpetable carrier.

In some embodiments, the compound of Formula (I) is selected from the group consisting of

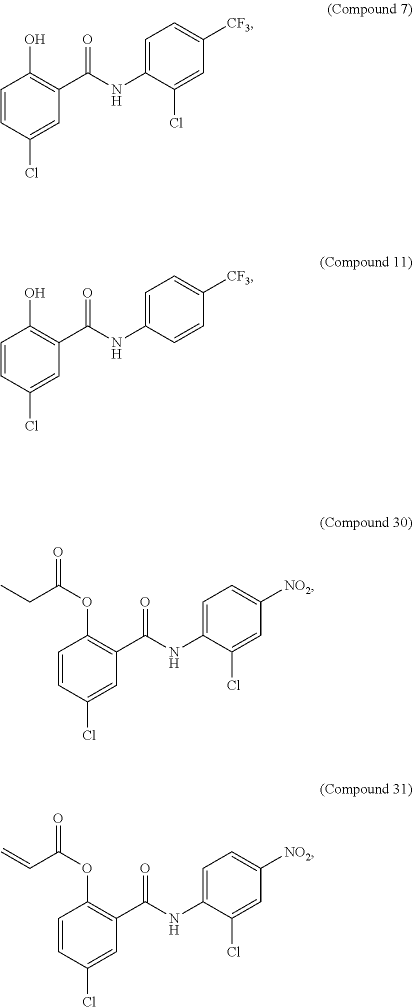 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.

In some embodiments, the antiandrogen drug is selected from the group consisting of a non-steroidal androgen receptor antagonist, a CYP17A1 inhibitor, and a combination thereof. In some instances, the antiandrogen drug is selected from the group consisting of bicalutamide, apalutamide, enzalutamide, abiraterone acetate, and a combination thereof.

In some embodiments, androgen receptor transactivation is inhibited. In some embodiments, androgen receptor expression is inhibited. In some embodiments, androgen receptor-mediated transcriptional activity is inhibited.

In some embodiments, the expression and/or activity of an androgen receptor variant is inhibited. In particular embodiments, recruitment of the androgen receptor variant to a prostate-specific antigen (PSA) promoter is inhibited. In some embodiments, the androgen receptor variant is selected from the group consisting of a splice variant, a mutant variant, and a combination thereof. In some instances, the splice variant is an AR-V1, AR-V3, AR-V7, AR-V9, and/or AR-V12 splice variant. In particular instances, the splice variant is an AR-V7 splice variant. In some instances, the mutant variant comprises one or more mutations selected from the group consisting of K581R, L702H, T878A, V716M, and a combination thereof relative to the amino acid sequence set forth in SEQ ID NO: 1.

In some embodiments, the cell is a cancer cell. In particular emboidments, the cancer cell is a metastatic cancer cell. In other embodiments, the cancer cell is a prostate cancer cell or a breast cancer cell. In some embodiments, the cancer cell is selected from the group consisting of an androgen-independent cancer cell, a castrate-resistant cancer cell, a hormone-resistant cancer cell, and a combination thereof. In some instances, the androgen independence, castrate resistance, and/or hormone resistance of the cancer cell is reduced, decreased, or reversed. In some embodiments, the cancer cell is resensitized to the antiandrogen drug. In other embodiments, resistance of the cancer cell to the antiandrogen drug is reduced, decreased, or reversed. In some embodiments, the invasive ability of the cancer cell and/or the ability of the cancer cell to migrate is inhibited. In other embodiments, the ability of the cancer cell to grow and/or form a colony is inhibited.

In another aspect, the invention provides a kit for preventing or treating cancer in a subject. In some embodiments, the kit comprises an antiandrogen drug and a compound according to Formula (I):

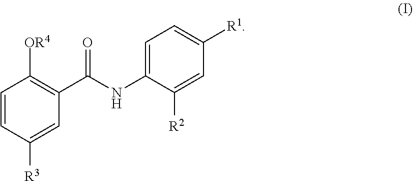

In particular embodiments, R¹ is selected from the group consisting of X, CX₃, NO₂, OH, and alkoxy; R² is selected from the group consisting of H, X, CX₃, NO₂, OH, and alkoxy; R³ is selected from the group consisting of X, CX₃, NO₂, OH, and alkoxy; and R⁴ is selected from the group consisting of H and C(O)R⁵, wherein R⁵ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R¹ is CX₃ or NO₂. In other instances, R² is H or X. In some other instances, R³ is X. In other instances, R⁵ is C₂ alkyl or C₂ alkenyl. In particular instances, X is independently selected from the group consisting of F and Cl. In particular embodiments, the kit further comprises a pharmaceutically accpetable carrier.

In some embodiments, the compound of Formula (I) is selected from the group consisting of

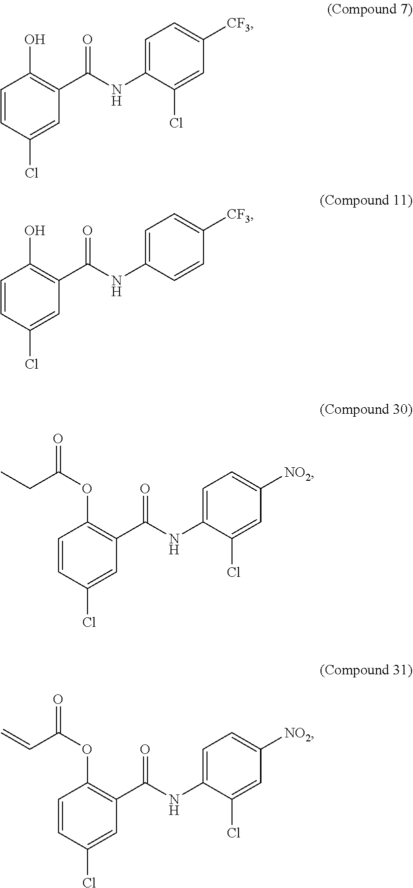 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.

In other embodiments, the antiandrogen drug is selected from the group consisting of a non-steroidal androgen receptor antagonist, a CYP17A1 inhibitor, and a combination thereof. In some instances, the antiandrogen drug is selected from the group consisting of bicalutamide, apalutamide, enzalutamide, abiraterone acetate, and a combination thereof.

In some embodiments, the cancer is prostate cancer or breast cancer. In other embodiments, the cancer is selected from the group consisting of an androgen-independent cancer, a metastatic cancer, a castrate-resistant cancer, a castration recurrent cancer, a hormone-resistant cancer, a metastatic castrate-resistant cancer, and a combination thereof.

In some other embodiments, the kit further comprises instructions for use. In some embodiments, the kit further comprises paraphernalia and/or one or more reagents for administering the antiandrogen drug and/or the compound of Formula (I) to the subject. In other embodiments, the kit further comprises paraphernalia and/or one or more reagents for obtaining a sample from the subject. In some embodiments, the kit further comprises paraphernalia and/or one or more reagents for determining the level of one or more biomarkers in the sample. In some instances, the one or more biomarkers comprises prostate-specific antigen (PSA). In some embodiments, the kit further comprises negative and/or positive control samples.

In another aspect, the invention provides a composition comprising an antiandrogen drug and a compound according to Formula (II):

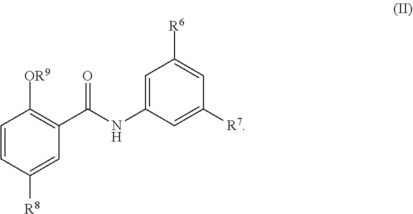

In particular embodiments, R⁶ and R⁷ are independently selected from the group consisting of H, X, CX₃, NO₂, OH, and alkoxy; R⁸ is selected from the group consisting of X, CX₃, NO₂, OH, and alkoxy; and R⁹ is selected from the group consisting of H and C(O)R¹⁰, wherein R¹⁰ is selected from the group consisting of H, optionally substituted C₁₋₁₈ alkyl, optionally substituted C₂₋₁₈ alkenyl, and optionally substituted C₂₋₁₈ alkynyl; and wherein X is an independently selected halogen. In some embodiments, R⁶ and/or R⁷ are CX₃. In some embodiments, R⁸ is X. In some embodiments, R⁹ is H. In particular embodiments, X is independently selected from the group consisting of F and Cl.

In some embodiments, the compound of Formula (II) is

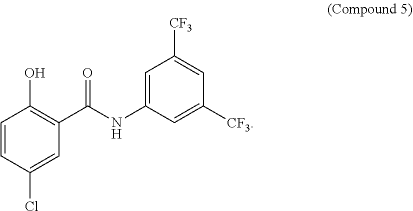 [SMILES: O=C(Nc1cc(C(F)(F)F)cc(C(F)(F)F)c1)c1cc(Cl)ccc1O]

In other embodiments, the antiandrogen drug is selected from the group consisting of a non-steroidal androgen receptor antagonist, a CYP17A1 inhibitor, and a combination thereof. In some instances, the antiandrogen drug is selected from the group consisting of bicalutamide, apalutamide, enzalutamide, abiraterone acetate, and a combination thereof.

In some embodiments, the composition inhibits the expression and/or activity of an androgen receptor or a variant thereof. In particular embodiments, the androgen receptor variant is selected from the group consisting of a splice variant, a mutant variant, and a combination thereof. In some instances, the splice variant is an AR-V1, AR-V3, AR-V7, AR-V9, and/or AR-V12 splice variant. In particular instances, the splice variant is an AR-V7 splice variant. In other instances, the mutant variant comprises one or more mutations selected from the group consisting of K581R, L702H, T878A, V716M, and a combination thereof relative to the amino acid sequence set forth in SEQ ID NO: 1.

In other embodiments, the composition is an effective inhibitor of cancer cell proliferation. In some instances, the cancer cell is a prostate cancer cell or a breast cancer cell. In other instances, the cancer cell is selected from the group consisting of an androgen-independent cancer cell, a metastatic cancer cell, a castrate-resistant cancer cell, a castration recurrent cancer cell, a hormone-resistant cancer cell, a metastatic castrate-resistant cancer cell, and a combination thereof.

In some embodiments, the composition further comprises a pharmaceutically acceptable carrier.

In still another aspect, the invention provides a method for preventing or treating cancer in a subject, the method comprising administering to the subject a therapeutically effective amount of a composition comprising an antiandrogen drug and a compound of Formula (II).

In yet another aspect, the invention provides a method for inhibiting the expression and/or activity of an androgen receptor in a cell, the method comprising contacting the androgen receptor or cell with a composition comprising an antiandrogen drug and a compound of Formula (II).

In another aspect, the invention provides a kit comprising a composition comprising an antiandrogen drug and a compound of Formula (II).

Other objects, features, and advantages of the present invention will be apparent to one of skill in the art from the following detailed description and figures.

### BRIEF DESCRIPTION OF THE DRAWINGS

FIGS. 1A-1C show the chemical structures and synthetic pathways of niclosamide analogs. FIG. 1A shows the synthetic pathways of Compounds 7 and 30. FIG. 1B shows the structures of niclosamide and Compounds 5, 7, 11, 30, and 31. FIG. 1C shows the structures of niclosamide and Compounds 1, 2, 5, 7, 8, 11, 17, 29, 30, 31, 34, and 35.

FIGS. 2A and 2B show that niclosamide analogs inhibited AR and AR-V7 expression. FIG. 2A shows a Western blot of lysates of CWR22Rv1 cells that were treated with niclosamide or one of the indicated compounds. FIG. 2B shows a Western blot of lysates of CWR22Rv1 cells that were treated with increasing doses of the indicated compounds. “AR-Variants” represents a combination of all forms of AR.

FIG. 3 shows that niclosamide analogs inhibited PSA expression. C4-2 V7 cells (i.e., C4-2 cells overexpressing AR-V7) were treated with niclosamide or one of the indicated compounds in the presence or absence of enzalutamide (MDV). PSA protein expression was determined by ELISA assay. * denotes p<0.05.

FIG. 4 shows that niclosamide analogs inhibited AR-V7-mediated transcriptional activity. LNCaP cells were co-transfected with PSA-luc reporter with or without AR-V7, and the transfected cells were then treated with enzalutamide, niclosamide or the indicated niclosamide analogs. The luciferase activity was then determined. * denotes p<0.05.

FIGS. 5A-5F show that niclosamide analogs suppressed mutant AR transactivation. HEK 293 cells were transiently transfected with expression vectors encoding the corresponding mutant AR and an androgen-responsive luciferase reporter gene construct. The cells were treated with the indicated antiandrogen drugs or niclosamide analogs. Luciferase activity was then determined. FIG. 5A shows the activation of wild-type AR. FIG. 5B shows the activation of AR-V7. FIG. 5C shows the activation of the T878A variant. FIG. 5D shows the activation of the K581R variant. FIG. 5E shows the activation of the V716M variant. FIG. 5F shows the activation of the L702H variant. * denotes p<0.05.

FIGS. 6A-6D show that niclosamide analogs inhibited cell growth in vitro. CWR22Rv1 and C4-2B MDVR cells were treated with either abiraterone, enzalutamide, ARN509, or increasing doses of niclosamide or one of Compounds 7 and 31 for 48 hours, after which time cell numbers were counted. FIG. 6A shows the results of a cell growth assay using CWR22Rv1 cells. FIG. 6B shows the results of a cell growth assay using C4-2B MDVR cells. FIG. 6C shows cell growth over time of CWR22Rv1 cells. FIG. 6D shows cell growth over time of C4-2B MDVR cells. * denotes p<0.05.

FIGS. 7A-7D show that niclosamide analogs inhibited cell growth in vitro. CWR22Rv1, C4-2B MDVR, C4-2B AbiR, and C4-2-V7 cells were treated with DMSO or 0.5 μM niclosamide or one of the niclosamide analogs in the presence or absence of enzalutamide (MDV) for 48 hours, after which time cell numbers were determined. FIG. 7A shows the results of a cell growth assay using CWR22Rv1 cells. FIG. 7B shows the results of a cell growth assay using C4-2B MDVR cells. FIG. 7C shows the results of a cell growth assay of C4-2B AbiR cells. FIG. 7D shows the results of a cell growth assay of C4-2-V7 cells. * denotes p<0.05.

FIG. 8 shows that niclosamide analogs induced apoptosis. C4-2BMDVR cells were treated with niclosamide or one of the indicated analogs, with or without enzalutamide (MDV), and apoptosis was measured. * denotes p<0.05.

FIGS. 9A-9D show that some but not all niclosamide analogs were able to synergize with antiandrogen drugs to inhibit CWR22Rv1 cancer cell growth. CWR22Rv1 cells were treated with the indicated niclosamide analog (0.25 μM), enzalutamide (20 μM), or abiraterone (5 μM), either alone or in combination. DMSO treatment was used as a negative control. Total cell numbers were counted at 0, 3, and 5 days. FIG. 9A shows the cell growth assay results when the niclosamide analog was Compound 1. FIG. 9B shows the cell growth assay results when the niclosamide analog was Compound 34. FIG. 9C shows the cell growth assay results when the niclosamide analog was Compound 30. FIG. 9D shows the cell growth assay results when the niclosamide analog was Compound 31. Compounds 30 and 31, but not Compounds 1 and 34, were able to synergize with enzalutamide and abiraterone. * denotes p<0.05.

FIGS. 10A-10D show that some but not all niclosamide analogs were able to synergize with the antiandrogen drugs enzalutamide and abiraterone to inhibit C4-2BMDVR cancer cell growth. C4-2BMDVR cells were treated with the indicated niclosamide analog (0.25 μM), enzalutamide (20 μM), or abiraterone (5 μM), either alone or in combination. DMSO treatment was used as a negative control. Total cell numbers were counted at 0, 3, and 5 days. FIG. 10A shows the cell growth assay results when the niclosamide analog was Compound 1. FIG. 10B shows the cell growth assay results when the niclosamide analog was Compound 34. FIG. 10C shows the cell growth assay results when the niclosamide analog was Compound 30. FIG. 10D shows the cell growth assay results when the niclosamide analog was Compound 31. Compounds 30 and 31, but not Compounds 1 and 34, were able to synergize with enzalutamide and abiraterone. * denotes p<0.05.

FIGS. 11A-11D show that some but not all niclosamide analogs were able to synergize with the antiandrogen drugs enzalutamide and abiraterone to inhibit CWR22Rv1 cancer cell growth. CWR22Rv1 cells were treated with the indicated niclosamide analog (0.25 μM), enzalutamide (20 μM), or abiraterone (5 μM), either alone or in combination. DMSO treatment was used as a negative control. Total cell numbers were counted at 0, 3, and 5 days. FIG. 11A shows the cell growth assay results when the niclosamide analog was Compound 7. FIG. 11B shows the cell growth assay results when the niclosamide analog was Compound 11. FIG. 11C shows the cell growth assay results when the niclosamide analog was Compound 2. FIG. 11D shows the cell growth assay results when the niclosamide analog was Compound 17. Compounds 7 and 11, but not Compounds 2 and 17, were able to synergize with enzalutamide and abiraterone. * denotes p<0.05.

FIGS. 12A-12D show that some but not all niclosamide analogs were able to synergize with the antiandrogen drug bicalutamide to inhibit CWR22Rv1 cancer cell growth. CWR22Rv1 cells were treated with the indicated niclosamide analog (0.25 μM), bicalutamide (20 μM), or a combination thereof. DMSO treatment was used as a negative control. Total cell numbers were counted at 0, 3, and 5 days. FIG. 12A shows the cell growth assay results when the niclosamide analog was Compound 1. FIG. 12B shows the cell growth assay results when the niclosamide analog was Compound 34. FIG. 12C shows the cell growth assay results when the niclosamide analog was Compound 31. FIG. 12D shows the cell growth assay results when the niclosamide analog was Compound 30. Compounds 30 and 31, but not Compounds 1 and 34, were able to synergize with bicalutamide. * denotes p<0.05.

FIGS. 13A-13D show that niclosamide analogs inhibited tumor growth and AR-V7 expression in vivo. CWR22Rv1 tumor xenografts were treated with niclosamide or Compound 7. Tumor volume, tumor weight, and body weight were measured. Protein lysates were isolated from the tumors and analyzed for AR and AR-V7 expression by Western blot. FIG. 13A shows a graph of CWR22Rv1 tumor xenograft volume as a function of time. FIG. 13B shows a graph of body weight for each of the three groups. FIG. 13C shows a picture of the tumors at the end of treatment. FIG. 13D shows a Western blot depicting full-length AR (AR-FL) and AR-V7 expression. “AR-Variants” represents a combination of all forms of AR. * denotes p<0.05.

FIGS. 14A-14F show that niclosamide and its analogs suppressed transcriptional activity of androgen receptor (AR) and its variants in fetal bovine serum, as assessed using a PSA-luc assay. FIG. 14A shows data for pcDnA. FIG. 14B shows data for AR-V1. FIG. 14C shows data for AR-V3. FIG. 14D shows data for AR-V7. FIG. 14E shows data for AR-V9. FIG. 14F shows data for AR-V12. Abbreviations: NIC, niclosamide; AA, abiraterone; ENZA, enzalutamide; ARN, apalutamide, #7, Compound 7; #31, Compound 31. * denotes p<0.05.

FIGS. 15A-15C show that niclosamide and its analogs degraded AR variants through proteasome-ubiquitination system activation in CWR22Rv1 cells. FIG. 15A shows the results of a Western blot (left) depicting AR variant protein degradation in CWR22Rv1 cells and relative protein levels (right). FIG. 15B shows a Western blot of CWR22Rv1 whole-cell lysate. FIG. 15C shows a Western blot following immunoprecipitation of CWR22Rv1 whole-cell lysate with an anti-AR antibody. Abbreviations: NIC, niclosamide; #7, Compound 7; #31, Compound 31. “AR-Variants” represents a combination of all forms of AR.

FIGS. 16A-16C show that niclosamide and its analogs degraded AR variants through proteasome-ubiquitination system activation in C4-2B MDVR cells. FIG. 16A shows the results of a Western blot (left) depicting AR variant protein degradation in C4-2B MDVR cells and relative protein levels (right). FIG. 16B shows a Western blot of C4-2B MDVR whole-cell lysate. FIG. 16C shows a Western blot following immunoprecipitation of C4-2B MDVR whole-cell lysate with an anti-AR antibody. Abbreviations: NIC, niclosamide; #7, Compound 7; #31, Compound 31. “AR-Variants” represents a combination of all forms of AR.

FIGS. 17A and 17B show that Compound 7 enhanced abiraterone and apalutamide (ARN509) treatment in resistant prostate cancer. FIG. 17A shows the results of treating CWR22Rv1 cells with apalutamide (ARN) and/or Compound 7 (#7). FIG. 17B shows the results of treating CWR22Rv1 cells with abiraterone (ABI) and/or Compound 7. * denotes p<0.05.

FIG. 18 shows that both Compound 7 (#7) and Compound 31 (#31) exhibited better bioavailability than niclosamide when administered orally.

FIGS. 19A-19D show that Compound 7 (#7) suppressed LuCaP 35CR PDX xenograft tumor growth and exhibited better anti-tumor activity than niclosamide (NIC) when orally administered. FIG. 19A shows tumor volume data (left) and images of tumors (right). FIG. 19B shows tumor weight data. FIG. 19C shows body weight data. FIG. 19D shows data for PSA levels in mouse serum samples. * denotes p<0.05.

FIGS. 20A and 20B show that Compound 7 (#7) and Compound 31 (#31) inhibited breast cancer cell growth. FIG. 20A shows data for MDA-MB-468 cells. FIG. 20B shows data for MCF-7 cells.

### DETAILED DESCRIPTION OF THE INVENTION

### I. INTRODUCTION

Development of cancer resistance to antiandrogen drugs such as enzalutamide and abiraterone is practically inevitable given that several potential pathways to the development of resistance are available. Recent studies have linked androgen receptor (AR) alternative splicing, particularly the splice variant AR-V7, to the development of cancer resistance to antiandrogen drugs. AR splice variants are associated with docetaxel resistance as well. Furthermore, it has been shown that AR-V7 expression in cancer patients treated with enzalutamide or abiraterone correlates to a significantly lower PSA response, shorter progression-free time, and lower overall survival time compared to patients who do not express AR-V7.

The present invention is based, in part, on the discovery of niclosamide analogs that can inhibit the growth of cancer cells, including androgen-independent prostate cancer cells expressing the androgen receptor variants AR-VI, AR-V3, AR-V7, AR-V9, and AR-V12. In addition, the invention is based, in part, on the surprising discovery that niclosamide analogs of the present invention can synergize with antiandrogen drugs in the inhibition of cancer cell growth. The compositions and methods of the present invention are useful for treating any number of cancers, including prostate cancers. Furthermore, the compositions and methods provided herein are useful for treating androgen-independent cancers and cancers that express androgen receptor splice variants such as AR-VI, AR-V3, AR-V7, AR-V9, and AR-V12, or any number of mutant variants of the androgen receptor.

### II. DEFINITIONS

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The terms “subject,” “individual,” and “patient” as used herein are used interchangeably herein to refer to a vertebrate, preferably a mammal, more preferably a human. Mammals include, but are not limited to, murines, rats, simians, humans, farm animals, sport animals, and pets. Tissues, cells and their progeny of a biological entity obtained in vivo or cultured in vitro are also encompassed.

As used herein, the term “therapeutically effective amount” includes a dosage sufficient to produce a desired result with respect to the indicated disorder, condition, or mental state. The desired result may comprise a subjective or objective improvement in the recipient of the dosage. For example, an effective amount of a niclosamide analog (e.g., Compound 5, 7, 11, 30, 31, or a combination thereof) and an antiandrogen drug (e.g., enzalutamide, apalutamide, abiraterone acetate, bicalutamide, or a combination thereof) includes an amount sufficient to alleviate the signs, symptoms, or causes of cancer, e.g. prostate or breast cancer. As another example, an effective amount of a niclosamide analog (e.g., Compound 5, 7, 11, 30, 31, or a combination thereof) and an anti-androgen drug (e.g., enzalutamide, apalutamide, abiraterone acetate, bicalutamide, or a combination thereof) includes an amount sufficient to alleviate the signs, symptoms, or causes of resistant, recurrent, or advanced cancer, e.g. androgen-independent, metastatic, castrate-resistant, castration recurrent, hormone-resistant, or metastatic castrate-resistant cancer. As another example, an effective amount of a niclosamide analog (e.g., Compound 5, 7, 11, 30, 31, or a combination thereof) and an anti-androgen drug (e.g., enzalutamide, apalutamide, abiraterone acetate, bicalutamide, or a combination thereof) includes an amount sufficient to prevent the development of a cancer.

Thus, a therapeutically effective amount can be an amount that slows, reverses, or prevents tumor growth, increases mean time of survival, inhibits tumor progression or metastasis, or re-sensitizes a cancer cell to a cancer drug to which it has become or is resistant (e.g., an antiandrogen drug such as enzalutamide, apalutamide, abiraterone acetate, or bicalutamide). Also, for example, an effective amount of a combination of a niclosamide analog and an antiandrogen drug includes an amount sufficient to cause a substantial improvement in a subject having cancer when administered to the subject. The effective mount can vary with the type and stage of the cancer being treated, the type and concentration of one or more compositions administered, and the amounts of other drugs that are also administered.

For example, the amount can vary with the type of cancer being treated, the stage of advancement of cancer, the type and concentration of one or more compositions applied, and the amounts of other drugs that are also administered to the subject. An effective amount of a niclosamide analog (e.g., Compound 5, 7, 11, 30, 31, or a combination thereof) and an anti-androgen drug (e.g., enzalutamide, apalutamide, abiraterone acetate, bicalutamide, or a combination thereof) can include an amount that is effective in enhancing the anti-cancer therapeutic activity of an antiandrogen drug such as enzalutamide, apalutamide, abiraterone acetate, or bicalutamide.

As used herein, the term “treating” includes, but is not limited to, methods and manipulations to produce beneficial changes in a recipient's health status, e.g., a patient's cancer status. The changes can be either subjective or objective and can relate to features such as symptoms or signs of the cancer being treated. For example, if the patient notes decreased pain, then successful treatment of pain has occurred. For example, if a decrease in the amount of swelling has occurred, then a beneficial treatment of inflammation has occurred. Similarly, if the clinician notes objective changes, such as reducing the number of cancer cells, the growth of the cancer cells, the size of cancer tumors, or the resistance of the cancer cells to another cancer drug (e.g., an anti-androgen drug such as enzalutamide, apalutamide, abiraterone acetate, or bicalutamide), then treatment of cancer has also been beneficial. Preventing the deterioration of a recipient's status is also included by the term. Treating, as used herein, also includes administering a combination of a niclosamide analog (e.g., Compound 5, 7, 11, 30, 31, or a combination thereof) and an antiandrogen drug (e.g., enzalutamide, apalutamide, abiraterone acetate, bicalutamide, or a combination thereof) to a patient having cancer (e.g., prostate cancer, breast cancer, androgen-independent cancer, metastatic cancer, castrate-resistant cancer, castration recurrent cancer, hormone-resistant cancer, or metastatic castrate-resistant cancer).

As used herein, the term “administering” includes activities associated with providing a patient an amount (e.g., a therapeutically effective amount) of a compound or composition described herein, e.g., a combination of a niclosamide analog and an antiandrogen drug. Administering includes providing unit dosages of compositions set forth herein to a patient in need thereof. Administering includes providing effect amounts of compounds or compositions described herein for specified period of time, e.g., for about 1, 2, 3, 4, 5, 6, 7, 8, 9, 10, 12, 13, 14, 15, 16, 17, 18, 19, 20, 21, 22, 23, 24, 25, 26, 27, 28, 29, 30, 60, 90, 120, or more days, or in a specified sequence, e.g., administration of a niclosamide analog (e.g., Compound 5, 7, 11, 30, 31, or a combination thereof) followed by the administration of an antiandrogen drug (e.g., enzalutamide, apalutamide, abiraterone acetate, bicalutamide, or a combination thereof), or vice versa.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the invention and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor, etc. One of skill in the art will recognize that other pharmaceutical carriers are useful in the present invention.

As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. The two or more structurally different compounds can be comprised of a niclosamide analog (e.g., Compound 5, 7, 11, 30, 31, or a combination thereof) and an antiandrogen drug (e.g., enzalutamide, apalutamide, abiraterone acetate, bicalutamide, or a combination thereof). In some instances, the co-administered compounds are administered by the same route. In other instances, the co-administered compounds are administered via different routes. For example, one compound can be administered orally, and the other compound can be administered, e.g., sequentially or simultaneously, via intravenous or intraperitoneal injection. The simultaneously or sequentially administered compounds or compositions can be administered such that at least one niclosamide analog and one antiandrogen drug are simultaneously present in a subject or in a cell at an effective concentration.

As used herein, the term “cancer” is intended to include any member of a class of diseases characterized by the uncontrolled growth of aberrant cells, The term includes all known cancers and neoplastic conditions, whether characterized as malignant, benign, recurrent, soft tissue, or solid, and cancers of all stages and grades including advanced, recurrent, pre- and post-metastatic cancers. Additionally, the term includes androgen-independent, castrate-resistant, castration recurrent, hormone-resistant, drug-resistant, and metastatic castrate-resistant cancers. Examples of different types of cancer include, but are not limited to, prostate cancer (e.g., prostate adenocarcinoma); breast cancers (e.g., triple-negative breast cancer, ductal carcinoma in situ, invasive ductal carcinoma, tubular carcinoma, medullary carcinoma, mucinous carcinoma, papillary carcinoma, cribriform carcinoma, invasive lobular carcinoma, inflammatory breast cancer, lobular carcinoma in situ, Paget's disease, Phyllodes tumors); gynecological cancers (e.g., ovarian, cervical, uterine, vaginal, and vulvar cancers); lung cancers non-small cell lung cancer, small cell lung cancer, mesothelioma, carcinoid tumors, lung adenocarcinoma); digestive and gastrointestinal cancers such as gastric cancer (e.g., stomach cancer), colorectal cancer, gastrointestinal stromal tumors (GIST), gastrointestinal carcinoid tumors, colon cancer, rectal cancer, anal cancer, bile duct cancer, small intestine cancer, and esophageal. cancer; thyroid cancer; gallbladder cancer; liver cancer; pancreatic cancer; appendix cancer; renal cancer (e.g., renal cell carcinoma), cancer of the central nervous system (e.g., glioblastoma, neuroblastoma); skin cancer (e.g., melanoma); bone and soft tissue sarcomas (e.g., Ewing's sarcoma); lymphomas; choriocarcinomas; urinary cancers (e.g., urothelial bladder cancer); head and neck cancers; and bone marrow and blood cancers (e.g., chronic lymphocytic leukemia, lymphoma). As used herein, a “tumor” comprises one or more cancerous cells.

As used herein, the terms “prostate cancer” and “prostate cancer cell” refer to a cancer cell or cells that reside in prostate tissue or are derived from prostate tissue. The prostate cancer can be benign, malignant, or metastatic. The prostate cancer can be androgen-insensitive, hormone-resistant, or castrate-resistant. The prostate cancer can be “advanced stage prostate cancer” or “advanced prostate cancer.” Advanced stage prostate cancer includes a class of prostate cancers that have progressed beyond early stages of the disease. Typically, advanced stage prostate cancers are associated with a poor prognosis. Types of advanced stage prostate cancers include, but are not limited to, metastatic prostate cancer, drug-resistant prostate cancer such as anti-androgen-resistant prostate cancer (e.g., enzalutamide-resistant prostate cancer, apalutamide-resistant prostate cancer, abiraterone-resistant prostate cancer, bicalutamide-resistant prostate cancer, and the like), taxane-resistant prostate cancer, hormone refractory prostate cancer, castrate-resistant prostate cancer, metastatic castrate-resistant prostate cancer, and combinations thereof. In some instances, the advanced stage prostate cancers do not generally respond, or are resistant, to treatment with one or more of the following conventional prostate cancer therapies: enzalutamide, abiraterone, bicalutamide, or apalutamide. Compounds, compositions, and methods of the present invention are provided for treating prostate cancer, such as advanced stage prostate cancer, including any one or more (e.g., two, three, four, five, six, seven, eight, nine, ten, or more) of the types of advanced stage prostate cancers disclosed herein.

As used herein, the phrase “enhancing the therapeutic effects” includes any of a number of subjective or objective factors indicating a beneficial response or improvement of the condition being treated as discussed herein. For example, enhancing the therapeutic effects of an antiandrogen drug (e.g., enzalutamide, apalutamide, abiraterone acetate, bicalutamide, or a combination thereof) includes re-sensitizing antiandrogen-resistant cancer (e.g., antiandrogen-resistant prostate or breast cancer) to antiandrogen therapy. Also, for example, enhancing the therapeutic effects of an antiandrogen drug (e.g., enzalutamide, apalutamide, abiraterone acetate, bicalutamide, or a combination thereof) includes altering antiandrogen-resistant cancer cells (e.g., antiandrogen-resistant prostate or breast cancer cells) so that the cells are not resistant to the antiandrogen drug (e.g., enzalutamide, apalutamide, abiraterone acetate, bicalutamide, or a combination thereof). Also, for example, enhancing the therapeutic effects of an antiandrogen drug (e.g., enzalutamide, apalutamide, abiraterone acetate, bicalutamide, or a combination thereof) includes additively or synergistically improving or increasing the activity of the antiandrogen drug. In some embodiments, the enhancement includes, or includes at least, about a one-fold, two-fold, three-fold, four-fold, five-fold, ten-fold, twenty-fold, fifty-fold, hundred-fold, or thousand-fold increase in the therapeutic activity of the antiandrogen drug used to treat cancer (e.g., prostate or breast cancer). In some embodiments, the enhancement includes, or includes at least, about a 10%, 20%, 30%, 40%, 50%, 60%, 75%, 80%, 90%, or 100% increase in the therapeutic activity (e.g., efficacy) of the antiandrogen used to treat cancer (e.g., prostate or breast cancer).

As used herein, the terms “reversing cancer cell resistance,” “reducing cancer cell resistance,” or “re-sensitizing cancer cell resistance” to a compound or drug includes altering or modifying a cancer cell that is resistant to a therapy such as antiandrogen therapy (e.g., enzalutamide, abiraterone, bicalutamide, or apalutamide) so that the cell is no longer resistant to antiandrogen therapy, or is less resistant to the antiandrogen therapy. As such, as used herein, the phrase “reversing prostate cancer cell resistance” to an antiandrogen includes altering or modifying a prostate cancer cell that is resistant to an antiandrogen (e.g., enzalutamide, abiraterone, bicalutamide, or apalutamide) therapy so that the cell is no longer resistant to antiandrogen therapy, or is less resistant to the antiandrogen therapy.

As used herein, the phrase “antiandrogen drug” or “antiandrogen” includes antiandrogen compounds that alter the androgen pathway by blocking the androgen receptors, competing for binding sites on the cell's surface, or affecting or mediating androgen production. Antiandrogens are useful for treating several diseases including, but not limited to, cancer (e.g., prostate cancer or breast cancer). Antiandrogen drugs include, but are not limited to, non-steroidal androgen receptor (AR) antagonists and CYP17A1 inhibitors (i.e., androgen synthesis inhibitors that are inhibitors of cytochrome P450 17A1). Non-steroidal AR antagonists include, as non-limiting examples, first-generation drugs (e.g., bicalutamide, flutamide, and nilutamide), second-generation drugs (e.g., apalutamide, darolutamide, and enzalutamide), and others such as cimetidine and topilutamide. Typically, non-steroidal AR antagonists are selective AR antagonists and have little to no antigonadotropic activity. Non-limiting examples of CYP17A1 inhibitors include abiraterone acetate, ketoconazole, and seviteronel.

As used herein, the term “AR variant” includes a splice variant or a mutant variant of full-length AR. The amino acid sequence of isoform 1 of the human AR is set forth in NCBI Reference Sequence NP_000035.2 (SEQ ID NO: 1). Various AR splice variants are known. See, Guo et al., Cancer Res. 2009 Mar 15;69(6):2305-13. Exemplary AR splice variants include, but are not limited to, AR-V1, AR-V3, AR-V7, AR-V9, and AR-V12, as well as variants lacking a functional ligand binding domain (LBD). An example of an AR splice variant that lacks an LBD is AR-V7. “AR-V7” includes androgen receptor splice variant 7, a contituitively active variant of an AR that lacks a functional LBD. See, e.g., Hu et al., Cancer Research, 69(1):16-22 (2009). Various AR mutant variants are also known. See, e.g., Marcelli et al., Cancer Research 60(4):944-949 (2000) and Brooke et al., Curr. Genomics, 10(1):18-25 (2009). AR mutations can result in, among other things, alterations in cofactor binding and/or decreased ligand specificity, both of which can confer a growth advantage to cancer cells. Mutations can occur at any number of positions within the AR. Non-limiting examples of AR mutant variants include those with amino acid substitutions at positions K581, L702, V716, T878, or any combination thereof, relative to the amino acid sequence set forth in SEQ ID NO: 1. The wild-type amino acid at a given position can be replaced with any other amino acid. In some instances, the AR mutant variants comprise one or more amino acid substitutions selected from the group consisting of K581R, L702H, V716M, and T878A.

Non-limiting examples of human amino acid sequences for AR-VI, AR-V3, AR-V7, and AR-V12 are set forth under NCBI Reference Sequences ACN39560.1, ACN39563.1, ACN39559.1, and ACZ81436.1, respectively. A con-1 imi ling, example of an AR-V9 amino acid sequence is set forth under SEQ ID NO: 2.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can refer to alkyl groups having up to 20 carbon atoms, such as, but not limited to heptyl, octyl, nonyl, decyl, etc. Alkyl groups can be optionally substituted with one or more moieties selected from halo, hydroxy, amino, alkylamino, alkoxy, haloalkyl, carboxy, amido, nitro, oxo, and cyano.

As used herein, the term “alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be optionally substituted with one or more moieties selected from halo, hydroxy, amino, alkylamino, alkoxy, haloalkyl, carboxy, amido, nitro, oxo, and cyano.

As used herein, the term “alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be optionally substituted with one or more moieties selected from halo, hydroxy, amino, alkylamino, alkoxy, haloalkyl, carboxy, amido, nitro, oxo, and cyano.

As used herein, the terms “halo” and “halogen” refer to fluorine, chlorine, bromine and iodine.

As used herein, the term “alkoxy” refers to an alkyl group having an oxygen atom that connects the alkyl group to the point of attachment: i.e., alkyl-O—. As for alkyl group, alkoxy groups can have any suitable number of carbon atoms, such as C₁₋₆ or C₁₋₄. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. Alkoxy groups can be optionally substituted with one or more moieties selected from halo, hydroxy, amino, alkylamino, alkoxy, haloalkyl, carboxy, amido, nitro, oxo, and cyano.

As used herein, the term “oxo” refers to an oxygen atom that is double-bonded to a compound (i.e., O═).

As used herein, the term “haloalkyl” refers to an alkyl moiety as defined above substituted with at least one halogen atom.

As used herein, the term “carboxy” refers to a moiety —C(O)OH. The carboxy moiety can be ionized to form the carboxylate anion.

As used herein, the term “amino” refers to a moiety —NR₃, wherein each R group is H or alkyl.

As used herein, the term “amido” refers to a moiety —NRC(O)R or —C(O)NR₂, wherein each R group is H or alkyl.

The term “synergy” or “synergistic effect” refers to an effect produced by two or more compounds (e.g., an antiandrogen drug or a niclosamide analog) that is greater than the effect produced by a sum of the effects of the individual compounds (i.e., an effect that is greater than an additive effect). Several methods are available for determining whether a combination of drugs produces a synergistic effect. As a non-limiting example, the Highest Single Agent approach simply reflects that the fact that the resulting effect of a combination of drugs (E_{AB}) is greater than the effects of the individual drugs (E_{A} and E_{B}). A combination index (CI) can be calculated according to the formula:

$$CI = \frac{max ( {E}_{A ,} {E}_{B} )}{{E}_{AB}} .$$

As another non-limiting example, according to the Response Additivity Approach, a synergistic drug combination effect occurs when the E_{AB} is greater than the expected additive effects of the individual drugs (E_{A} and E_{B}). Here, the CI is calculated using the formula:

$$CI = \frac{{E}_{A} + {E}_{B}}{{E}_{AB}} .$$

As yet another non-limiting example, the Bliss Independence model is based on the principle that drug effects are the outcomes of probabilistic processes, and makes the assumption that drugs act independently such that they do not interfere with each other (i.e., different sites of action). However, the model also assumes that each drug contributes to the production of a common result. According to this method, the observed combination effect is expressed as a probability (0<E_{AB}<1) and is compared to the expected additive effect expressed as

E_{A}+E_{B}(1−E_{A})=E_{A}+E_{B}−E_{A}E_{B},

where 0<E_{A}<1 and 0<E_{B}<1. The CI for this method is calculated using the formula:

$$CI = \frac{{E}_{A} + {E}_{B} - {E}_{A} {E}_{B}}{{E}_{AB}} .$$

Methods of identifying synergistic effects are further discussed in Foucquier J. and Guedj M. Pharmacology Research & Perspectives (2015) (3)3:e00149, incorporated herein by reference in its entirety for all purposes.

### III. DESCRIPTION OF THE EMBODIMENTS

A. Compositions

The present invention provides compositions comprising an antiandrogen drug and a niclosamide analog. Niclosamide ((5-chloro-N-2-chloro-4-nitro-phenyl)-2-hydroxybenzamide) is a Food and Drug Administration (FDA) approved drug effective against human tapeworms. The chemical structure of niclosamide is set forth below:

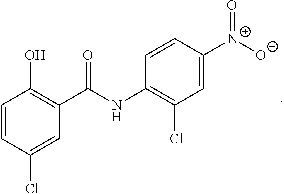 [SMILES: O=C(Nc1ccc([N+](=O)[O-])cc1Cl)c1cc(Cl)ccc1O]

As used herein, the term “niclosamide analog” includes structural analogs (i.e., compounds having structural similarity to niclosamide). Structural analogs of niclosamide can differ from niclosamide in one or more atoms, functional groups, or substructures. The term also includes derivatives of niclosamide analogs, as well as prodrugs that are converted to niclosamide analogs and derivatives thereof. Salts, such as pharmaceutically acceptable salts of niclosamide analogs, are also included.

In some embodiments, the niclosamide analog is a compound according to Formula (I):

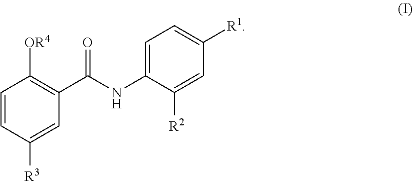

In some embodiments, R¹ is selected from the group consisting of X, CX₃, NO₂, OH, and alkoxy; R² is selected from the group consisting of H, X, CX₃, NO₂, OH, and alkoxy; R³ is selected from the group consisting of X, CX₃, NO₂, OH, and alkoxy; and R⁴ is selected from the group consisting of H and C(O)R⁵, wherein R⁵ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R¹ is CX₃ or NO₂. In other instances, R² is H or X. In some other instances, R³ is X. In other instances, R⁵ is C₂ alkyl or C₂ alkenyl. In particular instances, X is independently selected from the group consisting of F and Cl.

In some embodiments, R¹ is CX₃ (e.g., CF₃) and R² is H or X (e.g., Cl). In some embodiments, R¹ is CX₃, R³ is X (e.g., Cl), and R⁴ is H. In some embodiments, R¹ is CX₃ (e.g., CF₃), R² is H or X (e.g., Cl), R³ is X (e.g., Cl), and R⁴ is H. In some embodiments, R¹ is NO₂ and R⁴ is

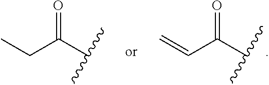

In some embodiments, R² is X (e.g., Cl) and R⁴ is

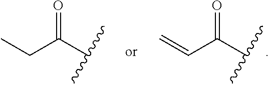

In some embodiments, R³ is X (e.g., Cl) and R⁴ is

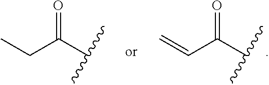

In some embodiments, R¹ is NO₂, R² is X (e.g., Cl), R³ is X (e.g., Cl), and R⁴ is

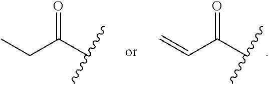

In some embodiments, R¹ is not NO₂ when R² is Cl, R³, is Cl, and R⁴ is H. In some embodiments, R² is not Cl when R¹ is NO₂, R³ is Cl, and R⁴ is H. In some embodiments, R³ is not Cl when R¹ is NO₂, R² is Cl, and R⁴ is H. In some embodiments, R⁴ is not H when R¹ is NO₂, R² is Cl, and R³ is Cl.

In particular embodiments, the compound of Formula (I) is selected from the group consisting of

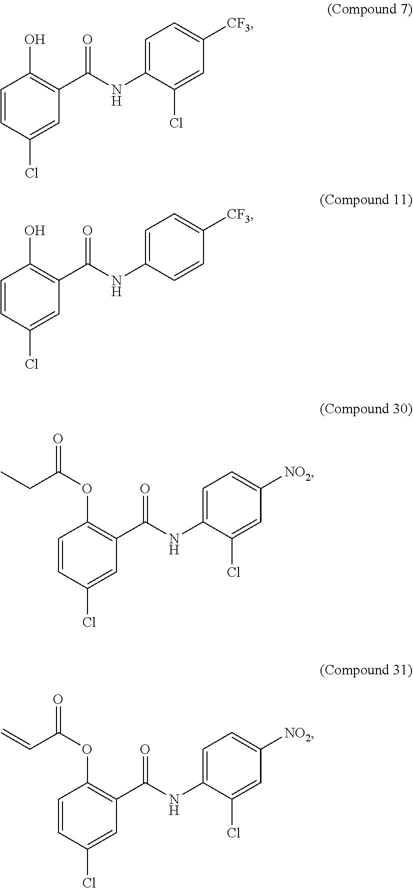 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.

In some embodiments, the niclosamide analog is a compound according to Formula (II):

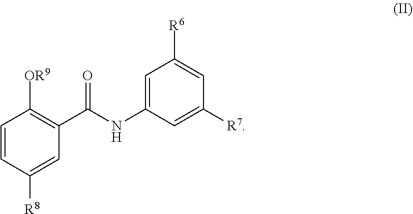

In some embodiments, R⁶ and R⁷ are independently selected from the group consisting of H, X, CX₃, NO₂, OH, and alkoxy; R⁸ is selected from the group consisting of X, CX₃, NO₂, OH, and alkoxy; and R⁹ is selected from the group consisting of H and C(O)R¹⁰, wherein R¹⁰ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R⁶ and/or R⁷ are CX₃. In some other instances, R⁸ is X. In some instances, R⁹ is H. In particular instances, X is independently selected from the group consisting of F and Cl.

In some embodiments, R⁶ and R⁷ are CX₃ (e.g., CF₃). In some embodiments, R⁶ and R⁷ are CX₃ (e.g., CF₃) and R⁸ is X (e.g., Cl). In some embodiments, R⁶ and R⁷ are CX₃ (e.g., CF₃) and R⁹ is H. In some embodiments, the compound of Formula (II) is

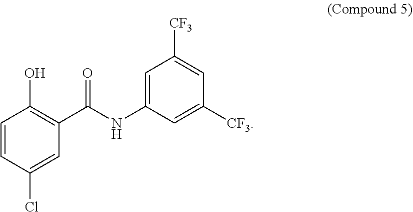 [SMILES: O=C(Nc1cc(C(F)(F)F)cc(C(F)(F)F)c1)c1cc(Cl)ccc1O]

In some embodiments, R⁶ is not F when R⁷ is F, R⁸ is Cl, and R⁹ is H. In some embodiments, R⁷ is not F when R⁶ is F, R⁸ is Cl, and R⁹ is H.

In some embodiments, the compound of Formula (I) or (II) is not niclosamide or Compound 1, 2, 8, 17, 29, 34, or 35.

In some embodiments, the niclosamide analog is a compound of Formula (I) and a compound of Formula (II).

In some embodiments, the compositions of the present invention include one or more niclosamide analogs at a concentration of between about 0.1 μM and 10 μM (e.g., about 0.1 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2 μM, 2.1 μM, 2.2 μM, 2.3 μM, 2.4 μM, 2.5 μM, 2.6 μM, 2.7 μM, 2.8 μM, 2.9 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, or 10 μM). In particular embodiments, the concentration is about 0.1 μM, 0.11 μM, 0.12 μM, 0.13 μM, 0.14 μM, 0.15 μM, 0.16 μM, 0.17 μM, 0.18 μM, 0.19 μM, 0.2 μM, 0.21 μM, 0.22 μM, 0.23 μM, 0.24 μM, 0.25 μM, 0.26 μM, 0.27 μM, 0.28 μM, 0.29 μM, 0.3 μM, 0.31 μM, 0.32 μM, 0.33 μM, 0.34 μM, 0.35 μM, 0.36 μM, 0.37 μM, 0.38 μM, 0.39 μM, or 0.4 μM. In other embodiments, the one or more niclosamide analogs are present at a concentration of between about 10 μM and 1,000 μM (e.g., about 10 μM, 11 μM, 12 μM, 13 μM, 14 μM, 15 μM, 16 μM, 17 μM, 18 μM, 19 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 150 μM, 200 μM, 250 μM, 300 μM, 350 μM, 400 μM, 450 μM, 500 μM, 550 μM, 600 μM, 650 μM, 700 μM, 750 μM, 800 μM, 850 μM, 900 μM, 950 μM, or 1,000 μM). In some instances, the niclosamide analog or a combination thereof is present in the composition at a concentration of about 0.25 μM. In some other instances, the niclosamide analog or a combination thereof is present in the composition at a concentration of about 0.5 μM. In other instances, the niclosamide analog or a combination thereof is present in the composition at a concentration of about 1.0 μM. In some other instances, the niclosamide analog or a combination thereof is present in the composition at a concentration of about 1.5 μM.

Compositions of the present invention comprise a niclosamide analog and an antiandrogen drug. Antiandrogen drugs include non-steroidal androgen receptor (AR) antagonists and CYP17A1 inhibitors. Suitable non-steroidal AR antagonists include bicalutamide (Casodex, Cosudex, Calutide, Kalumid), flutamide, nilutamide, apalutamide (ARN-509, JNJ-56021927), darolutamide, enzalutamide (Xtandi), cimetidine and topilutamide. Suitable CYP17A1 inhibitors include abiraterone acetate (Zytiga), ketoconazole, and seviteronel. Any combination of antiandrogen drugs can be used in compositions of the present invention.

In some embodiments, the compositions of the present invention include one or more antiandrogen drugs at a concentration of between about 0.1 μM and 10 μM (e.g., about 0.1 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2 μM, 2.1 μM, 2.2 μM, 2.3 μM, 2.4 μM, 2.5 μM, 2.6 μM, 2.7 μM, 2.8 μM, 2.9 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, or 10 μM). In other embodiments, the one or more antiandrogen drugs are present at a concentration of between about 10 μM and 100 μM (e.g., about 10 μM, 11 μM, 12 μM, 13 μM, 14 μM, 15 μM, 16 μM, 17 μM, 18 μM, 19 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM). In some other embodiments, the one or more antiandrogen drugs are present in the composition at a concentration between about 100 μM and 1,000 μM (e.g., about 100 μM, 150 μM, 200 μM, 250 μM, 300 μM, 350 μM, 400 μM, 450 μM, 500 μM, 550 μM, 600 μM, 650 μM, 700 μM, 750 μM, 800 μM, 850 μM, 900 μM, 950 μM, or 1,000 μM). In some instances, the antiandrogen drug or combination thereof is present at a concentration of about 5 μM. In other instances, the antiandrogen drug or combination thereof is present at a concentration of about 20 μM.

Compositions of the present invention are useful for inhibiting androgen receptor (AR) variants, including mutant variants and splice variants. In particular, many AR splice variants and mutant variants are associated with androgen-indepenent cancers and hormone-resistant cancers. Among AR mutant variants, compositions of the present invention are useful for inhibiting T878A, K581R, L702H and V716M AR mutant variants, as well as combinations thereof. Among AR splice variants, compositions of the present invention are useful as AR-V1, AR-V3, AR-V7, AR-V9, and AR-V12 splice variant inhibitors. In one aspect of this invention, inhibition of AR-V1, AR-V3, AR-V7, AR-V9, and/or AR-V12 can resensitize drug-resistant cancer cells (e.g., prostate cancer cells) to cancer drugs such as antiandrogen drugs (e.g., bicalutamide, enzalutamide, apalutmide and arbiraterone acetate). In another aspect of this invention, inhibition of AR-V1, AR-V3, AR-V7, AR-V9, and/or AR-V12 can enhance the effectiveness of antiandrogen drugs (e.g., bicalutamide, ell is resensiapalutamide, and arbiraterone acetate), and combinations thereof.

Compounds of the present invention can inhibit AR-V1, AR-V3, AR-V7, AR-V9, and/or AR-V12 transcription, translation, stability, or activity. Inhibition of AR-V1, AR-V3, AR-V7, AR-V9, and/or AR-V12 activity can include inhibition of recruitment of AR-V1, AR-V3, AR-V7, AR-V9, and/or AR-V12 to Androgen Response Elements (AREs). In some embodiments, inhibition of AR-V1, AR-V3, AR-V7, AR-V9, and/or AR-V12 activity can include inhibition of recruitment of AR-V1, AR-V3, AR-V7, AR-V9, and/or AR-V12 to the PSA promoter. In some embodiments, inhibition of AR-V1, AR-V3, AR-V7, AR-V9, and/or AR-V12 activity can include inhibition of AR-V1-, AR-V3-, AR-V7-, AR-V9-, and/or AR-V12-induced activation of the PSA promoter. In some embodiments, inhibition of AR-V1, AR-V3, AR-V7, AR-V9, and/or AR-V12 activity can include inhibition of AR-V1-, AR-V3-, AR-V7-, AR-V9-, and/or AR-V12-induced PSA production. For example, inhibition of AR-V1, AR-V3, AR-V7, AR-V9, and/or AR-V12 can include inhibition of production of PSA in the absence of DHT.

The present invention also provides such compositions which are tailored for patients having advanced stage cancer, such as drug-resistant cancer, metastatic cancer, castration-resistant cancer, or combinations thereof. In some instances, the advanced stage cancer is an advanced stage prostate cancer such as drug-resistant prostate cancer, metastatic prostate cancer, castration-resistant prostate cancer, or combinations thereof. In other instances, the advanced stage cancer is an advanced stage breast cancer, such as drug-resistant breast cancer, metastatic breast cancer, or a combination thereof.

B. Pharmaceutical Compositions

The pharmaceutical compositions of the present invention encompass compositions comprising a niclosamide analog (e.g., Compound 5, 7, 11, 30, 31, or a combination thereof), an antiandrogen drug (e.g., bicalutamide, apalutamide, enzalutamide, abiraterone acetate, or a combination thereof), and a pharmaceutically acceptable carrier and/or excipient or diluent. Such compositions are suitable for pharmaceutical use in an animal or human.

The pharmaceutical compositions of the present invention may be prepared by any of the methods well-known in the art of pharmacy. Pharmaceutically acceptable carriers suitable for use with the present invention include any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent.

The pharmaceutical compositions of the present invention can include a combination of drugs (e.g., a niclosamide analog such as Compound 5, 7, 11, 30, and/or 31 and an antiandrogen drug such as enzalutamide, abiraterone, bicalutamide, and/or apalutamide), or any pharmaceutically acceptable salts thereof, as active ingredients and a pharmaceutically acceptable carrier and/or excipient or diluent. A pharmaceutical composition may optionally contain other therapeutic ingredients.

The compositions (i.e., combinations of niclosamide analogs and antiandrogen drugs) of the present invention can be combined as the active ingredients in intimate admixture with a suitable pharmaceutical carrier and/or excipient according to conventional pharmaceutical compounding techniques. Any carrier and/or excipient suitable for the form of preparation desired for administration is contemplated for use with the compounds disclosed herein.

The pharmaceutical compositions include those suitable for topical, parenteral, pulmonary, nasal, rectal, or oral administration. The most suitable route of administration in any given case will depend in part on the nature and severity of the cancer (e.g., prostate or breast cancer) condition and also optionally the stage of the cancer.

Other pharmaceutical compositions include those suitable for systemic (enteral or parenteral) administration. Systemic administration includes oral, rectal, sublingual, or sublabial administration. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In particular embodiments, pharmaceutical compositions of the present invention may be administered intratumorally.

Compositions for pulmonary administration include, but are not limited to, dry powder compositions consisting of the powder of a compound described herein, or a salt thereof, and the powder of a suitable carrier and/or lubricant. The compositions for pulmonary administration can be inhaled from any suitable dry powder inhaler device known to a person skilled in the art.

Compositions for systemic administration include, but are not limited to, dry powder compositions consisting of the composition as set forth herein and the powder of a suitable carrier and/or excipient. The compositions for systemic administration can be represented by, but not limited to, tablets, capsules, pills, syrups, solutions, and suspensions.

In some embodiments, the present invention provides compositions further including a pharmaceutical surfactant. In other embodiments, the present invention provides compositions further including a cryoprotectant. In some embodiments, the cryoprotectant is selected from the group consisting of glucose, sucrose, trehalose, lactose, sodium glutamate, PVP, HPβCD, CD, glycerol, maltose, mannitol, and saccharose.

Pharmaceutical compositions or medicaments for use in the present invention can be formulated by standard techniques using one or more physiologically acceptable carriers or excipients. Suitable pharmaceutical carriers are described herein and in Remington: The Science and Practice of Pharmacy, 21st Ed., University of the Sciences in Philadelphia, Lippencott Williams & Wilkins (2005).

Controlled release parenteral formulations of the compositions of the present invention can be made as implants, oily injections, or as particulate systems. For a broad overview of delivery systems see, Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995) incorporated herein by reference. Particulate systems include microspheres, microparticles, microcapsules, nanocapsules, nanospheres, and nanoparticles.

Polymers can be used for ion-controlled release of compositions of the present invention. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has shown to be an effective vehicle for formulation and sustained delivery of recombinant interleukin 2 and urease (Johnston et al., Pharm. Res., 9:425-434 (1992); and Pec et al., J. Parent. Sci. Tech., 44(2):58 65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, PA (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

C. Methods for Preventing and Treating Cancer

In some embodiments, the present invention provides a method of preventing or treating cancer in a patient (e.g., prostate cancer, breast cancer, or an androgen-independent cancer), wherein the method comprises administering to the patient an effective amount of a niclosamide analog and an antiandrogen drug.

In some embodiments, the niclosamide analog is a compound according to Formula (I):

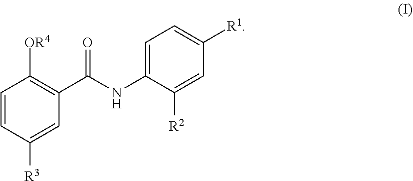

In some embodiments, R¹ is selected from the group consisting of X, CX₃, NO₂, OH, and alkoxy; R² is selected from the group consisting of H, X, CX₃, NO₂, OH, and alkoxy; R³ is selected from the group consisting of X, CX₃, NO₂, OH, and alkoxy; and R⁴ is selected from the group consisting of H and C(O)R⁵, wherein R⁵ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R¹ is CX₃ or NO₂. In other instances, R² is H or X. In some other instances, R³ is X. In other instances, R⁵ is C₂ alkyl or C₂ alkenyl. In particular instances, X is independently selected from the group consisting of F and Cl.

In some embodiments, R¹ is CX₃ (e.g., CF₃) and R² is H or X (e.g., Cl). In some embodiments, R¹ is CX₃, R³ is X (e.g., Cl), and R⁴ is H. In some embodiments, R¹ is CX₃ (e.g., CF₃), R² is H or X (e.g., Cl), R³ is X (e.g., Cl), and R⁴ is H. In some embodiments, R¹ is NO₂ and R⁴ is

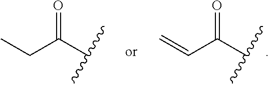

In some embodiments, R² is X (e.g., Cl) and R⁴ is

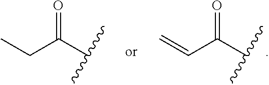

In some embodiments, R³ is X (e.g., Cl) and R⁴ is

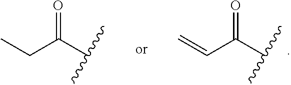

In some embodiments, R¹ is NO₂, R² is X (e.g., Cl), R³ is X (e.g., Cl), and R⁴ is

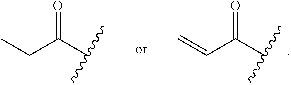

In some embodiments, R¹ is not NO₂ when R² is Cl, R³, is Cl, and R⁴ is H. In some embodiments, R² is not Cl when R¹ is NO₂, R³ is Cl, and R⁴ is H. In some embodiments, R³ is not Cl when R¹ is NO₂, R² is Cl, and R⁴ is H. In some embodiments, R⁴ is not H when R¹ is NO₂, R² is Cl, and R³ is Cl.

In particular embodiments, the compound of Formula (I) is selected from the group consisting of

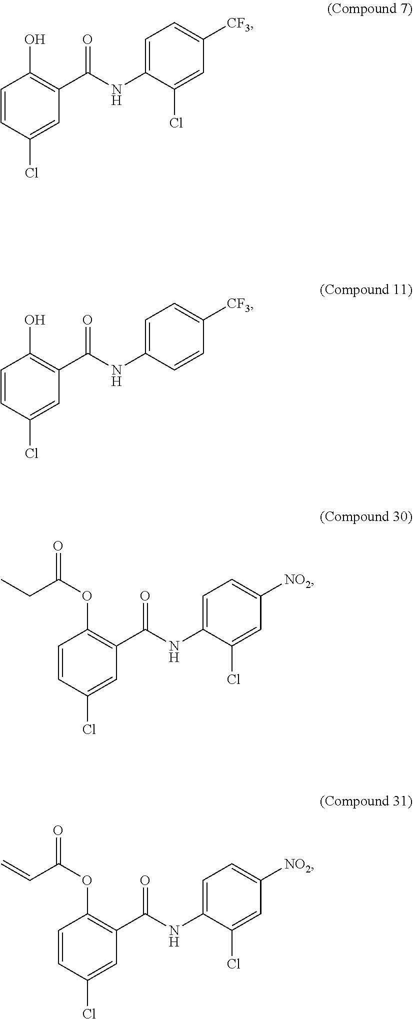 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.

In some embodiments, the niclosamide analog is a compound according to Formula (II):

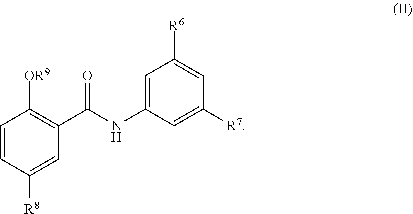

In some embodiments, R⁶ and R⁷ are independently selected from the group consisting of H, X, CX₃, NO₂, OH, and alkoxy; R⁸ is selected from the group consisting of X, CX₃, NO₂, OH, and alkoxy; and R⁹ is selected from the group consisting of H and C(O)R¹⁰, wherein R¹⁰ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R⁶ and/or R⁷ are CX₃. In some other instances, R⁸ is X. In some instances, R⁹ is H. In particular instances, X is independently selected from the group consisting of F and Cl.

In some embodiments, R⁶ and R⁷ are CX₃ (e.g., CF₃). In some embodiments, R⁶ and R⁷ are CX₃ (e.g., CF₃) and R⁸ is X (e.g., Cl). In some embodiments, R⁶ and R⁷ are CX₃ (e.g., CF₃) and R⁹ is H. In some embodiments, the compound of Formula (II) is

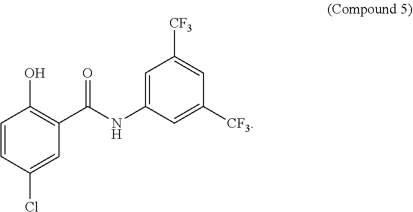 [SMILES: O=C(Nc1cc(C(F)(F)F)cc(C(F)(F)F)c1)c1cc(Cl)ccc1O]

In some embodiments, R⁶ is not F when R⁷ is F, R⁸ is Cl, and R⁹ is H. In some embodiments, R⁷ is not F when R⁶ is F, R⁸ is Cl, and R⁹ is H.

In some embodiments, the compound of Formula (I) or (II) is not niclosamide or Compound 1, 2, 8, 17, 29, 34, or 35.

In some embodiments, the niclosamide analog is a compound of Formula (I) and a compound of Formula (II).

In some embodiments, the antiandrogen drug is a non-steroidal AR antagonist, a CYP17A1 inhibitor, or a combination thereof. Suitable non-steroidal AR antagonists include bicalutamide (Casodex, Cosudex, Calutide, Kalumid), flutamide, nilutamide, apalutamide (ARN-509, JNJ-56021927), darolutamide, enzalutamide (Xtandi), cimetidine and topilutamide. Suitable CYP17A1 inhibitors include abiraterone acetate (Zytiga), ketoconazole, and seviteronel. Any combination of antiandrogen drugs can be used in methods of the present invention.

In some of these embodiments, the cancer is advanced stage cancer. In some of these embodiments, the cancer is drug resistant. In some of these embodiments, the cancer is antiandrogen drug resistant or androgen independent. In some of these embodiments, the cancer is metastatic. In some of these embodiments, the cancer is metastatic and drug resistant (e.g., antiandrogen drug resistant). In some of these embodiments, the cancer is castration resistant. In some of these embodiments, the cancer is metastatic and castration resistant. In some of these embodiments, the cancer is enzalutamide resistant. In some of these embodiments, the cancer is enzalutamide and arbiraterone resistant. In some of these embodiments, the cancer is enzalutamide, arbiraterone, and bicalutamide resistant. In some of these embodiments, the cancer is enzalutamide, arbiraterone, bicalutamide, and apalutamide resistant. In other embodiments, the cancer is resistant (e.g., docetaxel, cabazitaxel, paclitaxel). The cancer (e.g., prostate or breast cancer) can be resistant to any combination of these drugs.

In some embodiments, treatment comprises inhibiting cancer cell (e.g., prostate or breast cancer cell) growth, inhibiting cancer cell proliferation, inhibiting cancer cell migration, inhibiting cancer cell invasion, ameliorating the symptoms of cancer, reducing the size of a cancer tumor, reducing the number of cancer tumors, reducing the number of cancer cells, inducing cancer cell necrosis, pyroptosis, oncosis, apoptosis, autophagy, or other cell death, or enhancing the therapeutic effects of a composition or pharmaceutical composition comprising a niclosamide analog and an antiandrogen drug. In particular instances, the subject does not have cancer.

In particular methods of treating cancer (e.g., prostate cancer, breast cancer, androgen-independent cancer, or drug-resistant cancer), described herein, treatment comprises enhancing the therapeutic effects of an antiandrogen drug (e.g., a non-steroidal adrogen recept antagonist or a CYP17A1 inhibitor). In certain embodiments, treatment comprises enhancing the therapeutic effects of enzalutamide. In certain other embodiments, treatment comprises enhancing the therapeutic effects of abiraterone. In yet other embodiments, treatment comprises enhancing the therapeutic effects of apalutamide. In some other embodiments, treatment comprises enhancing the therapeutic effects of bicalutamide. The enhancement can be synergistic or additive.

In certain embodiments of the methods set forth herein, treatment comprises reversing, reducing, or decreasing cancer cell (e.g., prostate cancer cell or breast cancer cell) resistance to antiandrogen drugs. In certain embodiments of the methods set forth herein, treatment comprises resensitizing cancer cells (e.g., prostate cancer cells or breast cancer cells) to antiandrogen drugs. In any of the methods described herein, the antiandrogen drug is a compound selected from the group consisting of a non-steroidal androgen receptor antagonist, a CYP17A1 inhibitor, and a combination thereof. In certain embodiments, the antiandrogen drug is enzalutamide, apalutamide, bicalutamide, and/or abiraterone acetate.

In any of the aforementioned methods, treatment may comprise reversing cancer cell (e.g., prostate or breast cancer cell) resistance to an antiandrogen drug (e.g., a non-steroidal androgen receptor antagonist or CYP17A1 inhibitor); reducing or decreasing cancer cell resistance to an antiandrogen drug; or resensitizing cancer cells to an antiandrogen drug. In some embodiments, treatment comprises reversing cancer cell (e.g., prostate or breast cancer cell) resistance to enzalutamide, apalutamide, bicalutamide, abiraterone acetate, or a combination thereof. In some other embodiments, treatment comprises reducing or decreasing cancer cell resistance to enzalutamide, apalutamide, bicalutamide, abiraterone acetate, or a combination thereof. In yet other embodiments of the present invention, treatment comprises resensitizing cancer cells to enzalutamide, apalutamide, bicalutamide, abiraterone acetate, or a combination thereof.

In any of the methods described herein, the cancer is selected from the group consisting of castration-resistant cancer, metastatic castration-resistant cancer, advanced stage cancer, drug-resistant cancer, anti-androgen-resistant cancer, bicalutamide resistant cancer, enzalutamide-resistant cancer, abiraterone acetate-resistant cancer, apalutamide-resistant cancer, AR-V1-, AR-V3-, AR-V7-, AR-V9-, and/or AR-V12-induced drug-resistant cancer, AR-V1-, AR-V3-, AR-V7-, AR-V9-, and/or AR-V12-induced antiandrogen drug-resistant cancer, AR-VI-, AR-V3-, AR-V7-, AR-V9-, and/or AR-V12-induced enzalutamide-resistant cancer, AR-V1-, AR-V3-, AR-V7-, AR-V9-, and/or AR-V12-induced abiraterone acetate-resistant cancer, AR-V1-, AR-V3-, AR-V7-, AR-V9-, and/or AR-V12-induced apalutamide-resistant cancer, AR-V1-, AR-V3-, AR-V7-, AR-V9-, and/or AR-V12-induced bicalutamide-resistant cancer, and combinations thereof.

In particular embodiments, a test sample is obtained from the subject. The test sample can be obtained before and/or after the niclosamide analog(s) and antiandrogen drug(s) are administered to the subject. Non-limiting examples of suitable samples include blood, serum, plasma, cerebrospinal fluid, tissue, saliva, and urine. In some instances, the sample comprises normal tissue. In other instances, the sample comprises cancer tissue. The sample can also be made up of a combination of normal and cancer cells.

In some embodiments, a reference sample is obtained. The reference sample can be obtained, for example, from the subject and can comprise normal tissue. The reference sample can be also be obtained from a different subject and/or a population of subjects. In some instances, the reference sample is either obtained from the subject, a different subject, or a population of subjects before and/or after the niclosamide analog(s) and antiandrogen drug(s) are administered to the subject, and comprises normal tissue. However, in some instances the reference sample comprises cancer tissue and is obtained from the subject and/or from a different subject or a population of subjects.

In some embodiments, the level of one or more biomarkers is determined in the test sample and/or reference sample. Non-limiting examples of suitable biomarkers include prostate-specific antigen (PSA), alpha-methylacyl-CoA racemase (AMACR), endoglin (CD105), engrailed 2 (EN-2), prostate-specific membrane antigen (PSMA), caveolin-1, interleukin-6 (IL-6), CD147, members of the S100 protein family (e.g., S100A2, S100A4, S100A8, S100A9, S100A11), annexin A3 (ANXA3), human kallikrein-2 (KLK2), TGF-Betal, beta-microseminoprotein (MSMB), estrogen receptor (ER), progesterone receptor (PgR), HER2, Ki67, cyclin D1, and cyclin E.

Prostate-specific antigen (PSA) is a protein produced primarily by prostate cells. Most PSA is released into the semen, but some PSA is also released into the blood. In the blood, PSA exists in unbound and complexed (cPSA) forms. Conventional laboratory tests can measure unbound and/or total (unbound and complexed) PSA. Elevated PSA levels can be caused by benign prostatic hyperplasia (BPH) and inflammation of the prostate, but can also be caused by prostate cancer. Determining PSA levels may also include one or more determinations of PSA velocity (i.e., the change in PSA level over time), PSA doubling time (i.e., how quickly the PSA level doubles), PSA density (i.e., a comparison of the PSA concentration and the volume of the prostate (which can be evaluated, for example, by ultrasound)), and age-specific PSA ranges.

Typically, the level of the one or more biomarkers in one or more test samples is compared to the level of the one or more biomarkers in one or more reference samples. Depending on the biomarker, and increase or a decrease relative to a normal control or reference sample can be indicative of the presence of cancer or a higher risk for cancer. As a non-limiting example, levels of one or biomarkers in test samples taken before and after the niclosamide analog(s) and antiandrogen drug(s) are administered to the subject are compared to the level of the one or more biomarkers in a reference sample that is either normal tissue obtained from the subject, or normal tissue that is obtained from a different subject or a population of subjects. In some instances, the biomarker is serum, and the level of PSA in a test sample obtained from the subject before the niclosamide analog(s) and antiandrogen drug(s) are administered to the subject is higher than the level of PSA in the reference sample. In other instances, the level of PSA in a test sample obtained from the subject after administration of the niclosamide analog(s) and antiandrogen drug(s) is decreased relative to the level of PSA in a test sample obtained prior to administration. Alternatively, as another non-limiting example, the difference in PSA level between a sample obtained from the subject after administration and a reference sample is smaller than a difference between the PSA level in a sample obtained from the subject prior to administration and the reference sample (i.e., administration results in a decrease in PSA in the test sample such that the difference between the level measured in the test sample and the level measured in the reference sample is diminished or eliminated).

The differences between the reference sample or value and the test sample need only be sufficient to be detected. In some embodiments, an increased level of a biomarker (e.g., PSA) in the test sample, and hence the presence of cancer or increased risk of cancer, is determined when the biomarker levels are at least, e.g., about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold higher in comparison to a negative control. In other embodiments, a decreased level of a biomarker in the test sample, and hence the presence of cancer or increased risk of cancer, is determined when the biomarker levels are at least, e.g., about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold lower in comparison to a negative control.

The biomarker levels can be detected using any method known in the art, including the use of antibodies specific for the biomarkers. Exemplary methods include, without limitation, PCR, Western Blot, dot blot, enzyme-linked immunosorbent assay (ELISA), radioimmunoassay (RIA), immunoprecipitation, immunofluorescence, FACS analysis, electrochemiluminescence, and multiplex bead assays (e.g., using Luminex or fluorescent microbeads). In some instances, nucleic acid sequencing is employed.

In certain embodiments, the presence of decreased or increased levels of one or more biomarkers is indicated by a detectable signal (e.g., a blot, fluorescence, chemiluminescence, color, radioactivity) in an immunoassay or PCR reaction (e.g., quantitative PCR). This detectable signal can be compared to the signal from a control sample or to a threshold value. In some embodiments, a decreased presence is detected, and the presence or increased risk of cancer is indicated, when the detectable signal of biomarker(s) in the test sample is at least, e.g., about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold lower in comparison to the signal of antibodies in the reference sample or the predetermined threshold value. In other embodiments, an increased presence is detected, and the presence or increased risk of cancer is indicated, when the detectable signal of biomarker(s) in the test sample is at least, e.g., about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold greater in comparison to the signal of antibodies in the reference sample or the predetermined threshold value.

In some embodiments, the results of the biomarker level determinations are recorded in a tangible medium. For example, the results of diagnostic assays (e.g., the observation of the presence or decreased or increased presence of one or more biomarkers) and the diagnosis of whether or not there is an increased risk or the presence of cancer can be recorded, e.g., on paper or on electronic media (e.g., audio tape, a computer disk, a CD, a flash drive, etc.).

In other embodiments, the methods further comprise the step of providing the diagnosis to the patient (i.e., the subject) and/or the results of treatment.

D. Methods for Inhibiting Cancer Cells

In some embodiments, the present invention provides a method for inhibiting the expression and/or activity of an androgen receptor (e.g., a full-length androgen receptor, a wild-type androgen receptor, an androgen receptor variant, an androgen receptor mutant variant, or an androgen receptor splice variant) in a cell, wherein the method comprises contacting the cell or the androgen receptor with a combination of a niclosamide analog described herein and an antiandrogen drug described herein (e.g., a composition comprising a niclosamide analog and an antiandrogen drug).

In some embodiments, the niclosamide analog is a compound according to Formula (I):

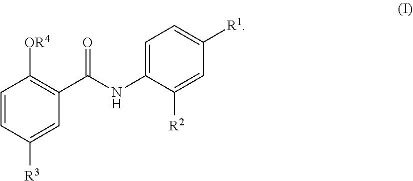

In some embodiments, R¹ is selected from the group consisting of X, CX₃, NO₂, OH, and alkoxy; R² is selected from the group consisting of H, X, CX₃, NO₂, OH, and alkoxy; R³ is selected from the group consisting of X, CX₃, NO₂, OH, and alkoxy; and R⁴ is selected from the group consisting of H and C(O)R⁵, wherein R⁵ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R¹ is CX₃ or NO₂. In other instances, R² is H or X. In some other instances, R³ is X. In other instances, R⁵ is C₂ alkyl or C₂ alkenyl. In particular instances, X is independently selected from the group consisting of F and Cl.

In some embodiments, R¹ is CX₃ (e.g., CF₃) and R² is H or X (e.g., Cl). In some embodiments, R¹ is CX₃, R³ is X (e.g., Cl), and R⁴ is H. In some embodiments, R¹ is CX₃ (e.g., CF₃), R² is H or X (e.g., Cl), R³ is X (e.g., Cl), and R⁴ is H. In some embodiments, R¹ is NO₂ and R⁴ is

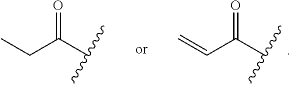

In some embodiments, R² is X (e.g., Cl) and R⁴ is

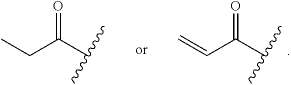

In some embodiments, R³ is X (e.g., Cl) and R⁴ is

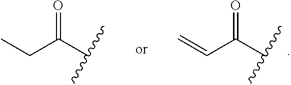

In some embodiments, R¹ is NO₂, R² is X (e.g., Cl), R³ is X (e.g., Cl), and R⁴ is

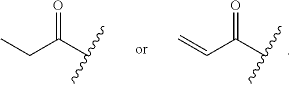

In some embodiments, R¹ is not NO₂ when R² is Cl, R³, is Cl, and R⁴ is H. In some embodiments, R² is not Cl when R¹ is NO₂, R³ is Cl, and R⁴ is H. In some embodiments, R³ is not Cl when R¹ is NO₂, R² is Cl, and R⁴ is H. In some embodiments, R⁴ is not H when R¹ is NO₂, R² is Cl, and R³ is Cl.

In particular embodiments, the compound of Formula (I) is selected from the group consisting of

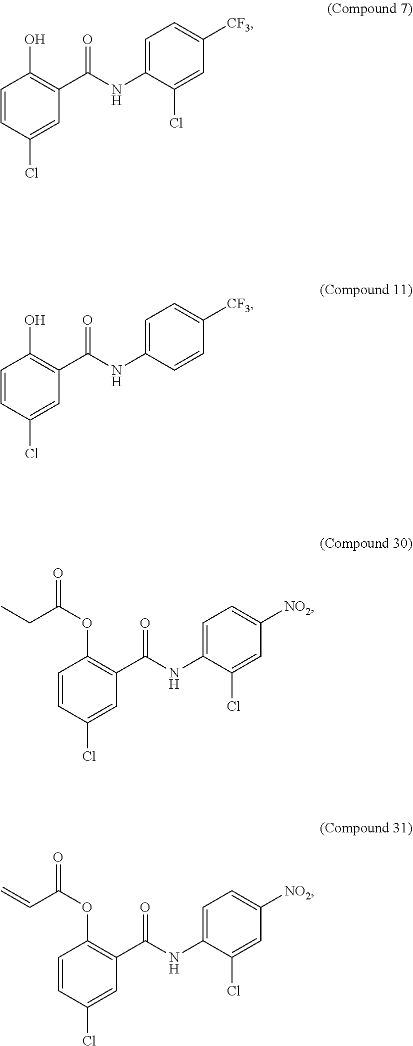 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.

In some embodiments, the niclosamide analog is a compound according to Formula (II):

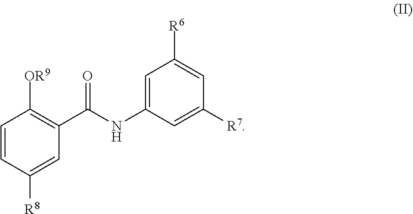

In some embodiments, R⁶ and R⁷ are independently selected from the group consisting of H, X, CX₃, NO₂, OH, and alkoxy; R⁸ is selected from the group consisting of X, CX₃, NO₂, OH, and alkoxy; and R⁹ is selected from the group consisting of H and C(O)R¹⁰, wherein R¹⁰ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R⁶ and/or R⁷ are CX₃. In some other instances, R⁸ is X. In some instances, R⁹ is H. In particular instances, X is independently selected from the group consisting of F and Cl.

In some embodiments, R⁶ and R⁷ are CX₃ (e.g., CF₃). In some embodiments, R⁶ and R⁷ are CX₃ (e.g., CF₃) and R⁸ is X (e.g., Cl). In some embodiments, R⁶ and R⁷ are CX₃ (e.g., CF₃) and R⁹ is H. In some embodiments, the compound of Formula (II) is

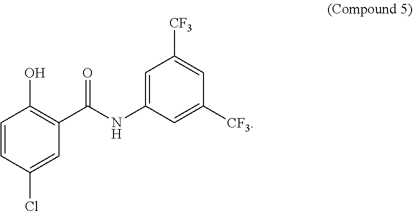 [SMILES: O=C(Nc1cc(C(F)(F)F)cc(C(F)(F)F)c1)c1cc(Cl)ccc1O]

In some embodiments, R⁶ is not F when R⁷ is F, R⁸ is Cl, and R⁹ is H. In some embodiments, R⁷ is not F when R⁶ is F, R⁸ is Cl, and R⁹ is H.

In some embodiments, the compound of Formula (I) or (II) is not niclosamide or Compound 1, 2, 8, 17, 29, 34, or 35.

In some embodiments, the niclosamide analog is a compound of Formula (I) and a compound of Formula (II).

In some embodiments, the antiandrogen drug is a non-steroidal AR antagonist, a CYP17A1 inhibitor, or a combination thereof. Suitable non-steroidal AR antagonists include bicalutamide (Casodex, Cosudex, Calutide, Kalumid), flutamide, nilutamide, apalutamide (ARN-509, JNJ-56021927), darolutamide, enzalutamide (Xtandi), cimetidine and topilutamide. Suitable CYP17A1 inhibitors include abiraterone acetate (Zytiga), ketoconazole, and seviteronel. Any combination of antiandrogen drugs can be used in methods of the present invention.

In certain embodiments, the variants are AR-V1, AR-V3, AR-V7, AR-V9, and/or AR-V12 splice variants. In some instances, the variants are AR-V7 splice variants. In certain other embodimetns, the variants are mutant variants comprising one or more mutations selected from the group consisting of K581R, L702H, T878A, and V716M relative to the amino acid sequence set forth in SEQ ID NO: 1. In certain embodiments, the amount is an effective amount or a therapeutically effective amount. In certain embodiments, the cell is a cancer cell, such as a castration-resistant cancer cell, an androgen independent or antiandrogen-resistant (e.g., enzalutamide-, apalutamide-, bicalutamide-, or abiraterone acetate-resistant) cancer cell, or a combination thereof. The cancer cell can be a prostate cancer cell, breast cancer cell, or other relevant cancer cell.

In some embodiments, the present invention provides a method for inhibiting cancer cell (e.g., prostate cancer or breast cancer cell) growth, wherein the method comprises contacting cancer cells with a niclosamide analog described herein such as Compound 5, 7, 11, 30, and/or 31 and an antiandrogen drug described herein such as enzalutamide, abiraterone acetate, apalutamide, and/or bicalutamide.

In some other embodiments, the present invention provides a method for inhibiting cancer cell (e.g., prostate or breast cancer cell) migration, wherein the method comprises contacting cancer cells with a niclosamide analog described herein such as Compound 5, 7, 11, 30, and/or 31 and an antiandrogen drug described herein such as enzalutamide, abiraterone acetate, apalutamide, and/or bicalutamide.

In some embodiments, the present invention provides a method for inhibiting cancer cell (e.g., prostate or breast cancer cell) invasion, wherein the method comprises contacting cancer cells with a niclosamide analog described herein such as Compound 5, 7, 11, 30, and/or 31 and an antiandrogen drug described herein such as enzalutamide, abiraterone acetate, apalutamide, and/or bicalutamide.

In some embodiments, the present invention provides a method for reversing cancer cell (e.g., prostate or breast cancer cell) resistance to antiandrogen drugs (such as enzalutamide, apalutamide, bicalutamide, abiraterone acetate, or a combination thereof), wherein the method comprises contacting cancer cells with a niclosamide analog described herein such as Compound 5, 7, 11, 30, and/or 31 and an antiandrogen drug described herein such as enzalutamide, abiraterone acetate, apalutamide, and/or bicalutamide. In certain emodiments, the amount is an effective amount or a therapetucially effective amount.

In some embodiments, the present invention provides a method for resensitizing cancer cells (e.g., prostate or breast cancer cells) to antiandrogen drugs such as enzalutamide, bicalutamide, apalutamide, abiraterone acetate, or a combination thereof), wherein the method comprises contacting cancer cells with a niclosamide analog described herein such as Compound 5, 7, 11, 30, and/or 31 and an antiandrogen drug described herein such as enzalutamide, abiraterone acetate, apalutamide, and/or bicalutamide. In certain emodiments, the amount is an effective amount or a therapeutically effective amount.

In some embodiments, the present invention provides a method for reducing or decreasing cancer cell (e.g., prostate cancer cell) resistance to antiandrogen drugs such as enzalutamide, bicalutamide, apalutamide, abiraterone acetate, or a combination thereof, wherein the method comprises contacting cancer cells with a niclosamide analog described herein such as Compound 5, 7, 11, 30, and/or 31 and an antiandrogen drug described herein such as enzalutamide, abiraterone acetate, apalutamide, and/or bicalutamide.

In some embodiments set forth herein, the reducing, reversing, or decreasing cancer cell (e.g., prostate or breast cancer cell) resistance to antiandrogen drugs or resensitizing the cancer cell to antiandrogen drugs occurs in a patient having cancer (e.g., prostate or breast cancer), although any number of other suscepticle cancers, including androgen-independent cancers, are appropriate for the methods of the present invention.

In some embodiments, the present invention also provides a method for enhancing the therapeutic effects of an antiandrogen drug (e.g., enzalutamide, apalutamide, bicalutamide, abiraterone acetate, or a combination thereof) in a patient having cancer (e.g., prostate or breast cancer), wherein the method comprises contacting cancer cells with or administering to a patient a niclosamide analog described herein such as Compound 5, 7, 11, 30, and/or 31 and an antiandrogen drug described herein such as enzalutamide, abiraterone acetate, apalutamide, and/or bicalutamide.

In other embodiments, the present invention provides a method for inhibiting an androgen receptor (AR) splice variant, comprising contacting an AR splice variant with a niclosamide analog described herein such as Compound 5, 7, 11, 30, and/or 31 and an antiandrogen drug described herein such as enzalutamide, abiraterone acetate, apalutamide, and/or bicalutamide. In some of these embodiments, the AR splice variant is AR-VI, AR-V3, AR-V7, AR-V9, and/or AR-V12. In some instances, the AR splice variant is AR-V7.

In other embodiments, the present invention provides a method for inhibiting an androgen receptor (AR) mutant variant, comprising contacting an AR mutant variant with a niclosamide analog described herein such as Compound 5, 7, 11, 30, and/or 31 and an antiandrogen drug described herein such as enzalutamide, abiraterone acetate, apalutamide, and/or bicalutamide. In some embodiments, the AR mutant variant comprises one or more mutations at positions selected from the gorup consisting of K581, L7602, T878, V716, and a combination thereof relative to the amino acid sequence set forth in SEQ ID NO: 1. In some instances, the AR mutant variant comprises one or more mutations selected from the group consisting of K581R, L702H, T878A, V716M, and a combination thereof relative to the amino acid sequence set forth in SEQ ID NO: 1.

In certain other embodiments, the present invention provides a method for inhibiting AR transactivation, inhibiting AR expression (e.g., mRNA expression and/or protein expression), inhibiting AR-mediated transcriptional activity, inhibiting AR-mediated cell migration, inhibiting AR-mediated cell invasion in cancer cells, inhibiting cancer cell colony formation, and inhibiting recruitment of an AR variant to a prostate-specific antigen (PSA) promoter. In certain instances, this method of inhibition comprises contacting cancer cells with a niclosamide analog described herein such as Compound 5, 7, 11, 30, and/or 31 and an antiandrogen drug described herein such as enzalutamide, abiraterone acetate, apalutamide, and/or bicalutamide.

The present invention also provides a method for inhibiting AR full length, AR-V1, AR-V3, AR-V7, AR-V9, and/or AR-V12 expression (e.g., mRNA and/or protein expression), wherein the method comprises contacting an AR or a cancer cell with a niclosamide analog described herein such as Compound 5, 7, 11, 30, and/or 31 and an antiandrogen drug described herein such as enzalutamide, abiraterone acetate, apalutamide, and/or bicalutamide.

The present invention further provides a method for inhibiting AR full length, AR-V1-, AR-V3-, AR-V7-, AR-V9-, and/or AR-V12-mediated transcriptional activity, wherein the method comprises contacting an AR or a cancer cell with a niclosamide analog described herein such as Compound 5, 7, 11, 30, and/or 31 and an antiandrogen drug described herein such as enzalutamide, abiraterone acetate, apalutamide, and/or bicalutamide.

In certain other embodiments, the present invention provides a method for inhibiting androgen-independent or antiandrogen drug-resistant CRPC cell growth, migration or invasion, wherein the method comprises contacting a prostate cancer cell with a niclosamide analog described herein such as Compound 5, 7, 11, 30, and/or 31 and an antiandrogen drug described herein such as enzalutamide, abiraterone acetate, apalutamide, and/or bicalutamide. In some embodiments, the prostate cancer cell is a CRPC cell.

E. Administration

In some embodiments of the present invention, a combination of a niclosamide analog and an antiandrogen drug is administered to a patient having cancer. The cancer can be any susceptible cancer. In some instances, the cancer is an androgen-independent cancer, a prostate cancer, or a breast cancer. In some embodiments, the niclosamide analog has Formula (I):

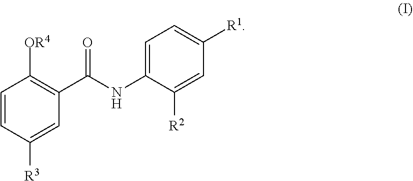

In some embodiments, R¹ is selected from the group consisting of X, CX₃, NO₂, OH, and alkoxy; R² is selected from the group consisting of H, X, CX₃, NO₂, OH, and alkoxy; R³ is selected from the group consisting of X, CX₃, NO₂, OH, and alkoxy; and R⁴ is selected from the group consisting of H and C(O)R⁵, wherein R⁵ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R¹ is CX₃ or NO₂. In other instances, R² is H or X. In some other instances, R³ is X. In other instances, R⁵ is C₂ alkyl or C₂ alkenyl. In particular instances, X is independently selected from the group consisting of F and Cl.

In some embodiments, R¹ is CX₃ (e.g., CF₃) and R² is H or X (e.g., Cl). In some embodiments, R¹ is CX₃, R³ is X (e.g., Cl), and R⁴ is H. In some embodiments, R¹ is CX₃ (e.g., CF₃), R² is H or X (e.g., Cl), R³ is X (e.g., Cl), and R⁴ is H. In some embodiments, R¹ is NO₂ and R⁴ is

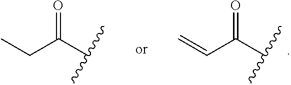

In some embodiments, R² is X (e.g., Cl) and R⁴ is

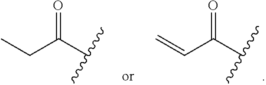

In some embodiments, R³ is X (e.g., Cl) and R⁴ is

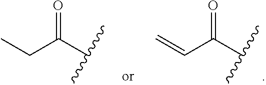

In some embodiments, R¹ is NO₂, R² is X (e.g., Cl), R³ is X (e.g., Cl), and R⁴ is

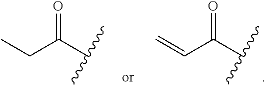

In some embodiments, R¹ is not NO₂ when R² is Cl, R³, is Cl, and R⁴ is H. In some embodiments, R² is not Cl when R¹ is NO₂, R³ is Cl, and R⁴ is H. In some embodiments, R³ is not Cl when R¹ is NO₂, R² is Cl, and R⁴ is H. In some embodiments, R⁴ is not H when R¹ is NO₂, R² is Cl, and R³ is Cl.

In particular embodiments, the niclosamide analog is selected from the group consisting of

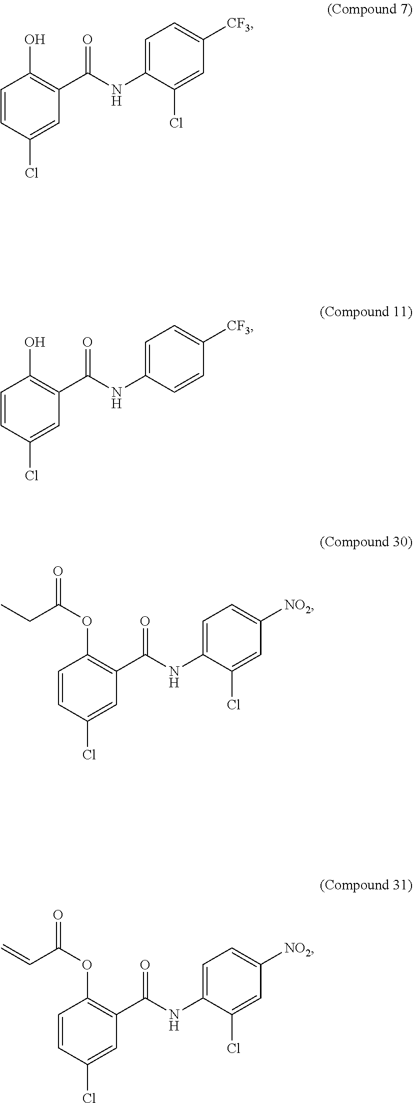 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.

In some embodiments, the niclosamide analog is a compound according to Formula (II):

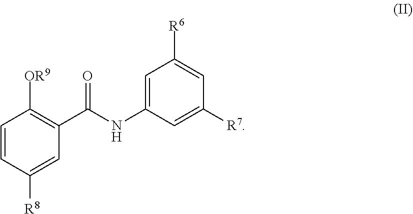

In some embodiments, R⁶ and R⁷ are independently selected from the group consisting of H, X, CX₃, NO₂, OH, and alkoxy; R⁸ is selected from the group consisting of X, CX₃, NO₂, OH, and alkoxy; and R⁹ is selected from the group consisting of H and C(O)R¹⁰, wherein R¹⁰ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R⁶ and/or R⁷ are CX₃. In some other instances, R⁸ is X. In some instances, R⁹ is H. In particular instances, X is independently selected from the group consisting of F and Cl.

In some embodiments, R⁶ and R⁷ are CX₃ (e.g., CF₃). In some embodiments, R⁶ and R⁷ are CX₃ (e.g., CF₃) and R⁸ is X (e.g., Cl). In some embodiments, R⁶ and R⁷ are CX₃ (e.g., CF₃) and R⁹ is H. In some embodiments, the compound of Formula (II) is

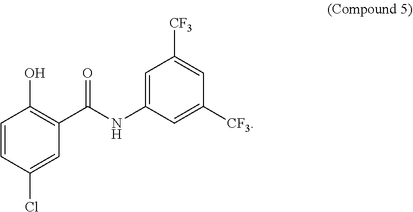 [SMILES: O=C(Nc1cc(C(F)(F)F)cc(C(F)(F)F)c1)c1cc(Cl)ccc1O]

In some embodiments, R⁶ is not F when R⁷ is F, R⁸ is Cl, and R⁹ is H. In some embodiments, R⁷ is not F when R⁶ is F, R⁸ is Cl, and R⁹ is H.

In some embodiments, the compound of Formula (I) or (II) is not niclosamide or Compound 1, 2, 8, 17, 29, 34, or 35.

In some embodiments, the niclosamide analog is a compound of Formula (I) and a compound of Formula (II).

In some embodiments, the antiandrogen drug is a non-steroidal AR antagonist, a CYP17A1 inhibitor, or a combination thereof. Suitable non-steroidal AR antagonists include bicalutamide (Casodex, Cosudex, Calutide, Kalumid), flutamide, nilutamide, apalutamide (ARN-509, JNJ-56021927), darolutamide, enzalutamide (Xtandi), cimetidine and topilutamide. Suitable CYP17A1 inhibitors include abiraterone acetate (Zytiga), ketoconazole, and seviteronel. Any combination of antiandrogen drugs can be used in methods of the present invention.

In certain methods of treating prostate cancer set forth herein, the methods comprise first administering a niclosamide analog to a patient having cancer, and then administering an antiandrogen drug to the patient. In certain methods of treating cancer set forth herein, the methods comprise first administering an antiandrogen drug to a patient having cancer, and then administering a niclosamide analog to the patient.

In some embodiments, the present invention provides a method of delivering an effective amount or a therapeutically effective amount of a niclosamide analog and an antiandrogen drug to a patient having cancer.

The niclosamide analog and antiandrogen drug formulations of the present invention are useful in the manufacture of a pharmaceutical composition or a medicament. A pharmaceutical composition or medicament can be administered to a subject in need thereof, e.g. a patient having cancer or at risk for cancer.

In any of the aforementioned embodiments, the cancer is selected from the group consisting of castration-resistant cancer, metastatic castration-resistant cancer, advanced stage cancer, androgen-independent cancer, drug-resistant cancer such as antiandrogen-resistant prostate cancer (e.g., enzalutamide-resistant cancer, abiraterone-resistant cancer, bicalutamide-resistant cancer, abiraterone acetate-resistant cancer, and the like), AR-V1-, AR-V3-, AR-V7-, AR-V9-, and/or AR-V12-induced antiandrogen-resistant cancer such as AR-V1-, AR-V3-, AR-V7-, AR-V9-, and AR-V12-induced enzalutamide-, apalutamide-, bicalutamide-, or abiraterone acetate-resistant cancer, and combinations thereof.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E. W. Martin. Compounds and agents of the present invention and their physiologically acceptable salts and solvates can be formulated for administration by any suitable route, including via inhalation, topically, nasally, orally, intravenously, parenterally, rectally, or intratumorally.

1. Routes of Administration

Typical formulations for topical administration include creams, ointments, sprays, lotions, and patches. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral or rectal administration is also contemplated.

Suitable formulations for transdermal application include an effective amount of one or more compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

For oral administration, a pharmaceutical composition or a medicament can take the form of, for example, a tablet or a capsule prepared by conventional means with a pharmaceutically acceptable caner or excipient. The present invention provides tablets and gelatin capsules comprising a niclosamide analog and an antiandrogen drug, or a dried solid powder of these drugs, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound(s).

The compositions and formulations set forth herein can be formulated for parenteral administration by injection, for example by bolus injection or continuous infusion. Formulations for injection can be presented in unit dosage form, for example, in ampules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively.

For administration by inhalation, the compositions of the present invention may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions set forth herein can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

In particular embodiments, a pharmaceutical composition or medicament of the present invention can comprise (i) an effective amount of a niclosamide analog, and (ii) a an antiandrogen drug. The therapeutic agent(s) may be used sequentially, or concomitantly. Administration may be by the same or different route of administration or together in the same pharmaceutical formulation.

2. Dosage

Pharmaceutical compositions or medicaments can be administered to a subject at a therapeutically effective dose to prevent, treat, resensitize, or control cancer as described herein. The pharmaceutical composition or medicament is administered to a subject in an amount sufficient to elicit an effective therapeutic response in the subject.

In some embodiments, the dosage of active agents administered is dependent on the subject's body weight, age, individual condition, surface area or volume of the area to be treated and on the form of administration. The size of the dose also can be determined by the existence, nature, and extent of any adverse effects that accompany the administration of a particular formulation in a particular subject. A unit dosage for oral administration to a mammal of about 50 to about 70 kg may contain between about 5 and about 500 mg, about 25 and about 200 mg, about 100 and about 1000 mg, about 200 and about 2000 mg, about 500 and about 5000 mg, or between about 1000 and about 2000 mg of one or more active ingredients. A unit dosage for oral administration to a mammal of about 50 to about 70 kg may contain about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,875 mg, 1,900 mg, 1,950 mg, 2,000 mg, 2,050 mg, 2,100 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg, 3,000 mg, 4,000 mg, 4,500 mg, 5,000, 5,500 mg, 6,000 mg, 6,500 mg, 7,000 mg, 7,500 mg, 8,000 mg, 8,500 mg, 9,000 mg, 9,500 mg, 10,000 mg, or more of one or more active ingredients (e.g., niclosamide analog(s), antiandrogen drug(s), or additional active ingredients). Typically, a dosage of the active compound(s) of the present invention is a dosage that is sufficient to achieve the desired effect. Optimal dosing schedules can be calculated from measurements of active agent accumulation in the body of a subject. In general, dosage may be given once or more of daily, weekly, or monthly. Persons of ordinary skill in the art can easily determine optimum dosages, dosing methodologies and repetition rates.

In some embodiments, the composition contains between about 0.1 mg/kg and about 500 mg/kg or more (e.g., about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.5 mg/kg, 3 mg/kg, 3.5 mg/kg, 4 mg/kg, 4.5 mg/kg, 5 mg/kg mg/kg, 5.5 mg/kg, 6 mg/kg. 6.5 mg/kg, 7 mg/kg, 7.5 mg/kg, 8 mg/kg, 8.5 mg/kg, 9 mg/kg, 9.5 mg/kg 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg/kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, 500 mg/kg, or more) of niclosamide analog(s).

In other embodiments, the composition contains between about 0.1 mg/kg and about 500 mg/kg or more (e.g., about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.5 mg/kg, 3 mg/kg, 3.5 mg/kg, 4 mg/kg, 4.5 mg/kg, 5 mg/kg mg/kg, 5.5 mg/kg, 6 mg/kg. 6.5 mg/kg, 7 mg/kg, 7.5 mg/kg, 8 mg/kg, 8.5 mg/kg, 9 mg/kg, 9.5 mg/kg 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg/kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, 500 mg/kg, or more) of antiandrogen drug(s).

In other embodiments, the composition contains between about 0.1 mg/kg and about 500 mg/kg or more (e.g., about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.5 mg/kg, 3 mg/kg, 3.5 mg/kg, 4 mg/kg, 4.5 mg/kg, 5 mg/kg mg/kg, 5.5 mg/kg, 6 mg/kg. 6.5 mg/kg, 7 mg/kg, 7.5 mg/kg, 8 mg/kg, 8.5 mg/kg, 9 mg/kg, 9.5 mg/kg 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg/kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, 500 mg/kg, or more) of a combination of niclosamide analog(s) and antiandrogen drug(s).

A unit dosage may contain about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,875 mg, 1,900 mg, 1,950 mg, 2,000 mg, 2,050 mg, 2,100 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg, 3,000 mg, 4,000 mg, 4,500 mg, 5,000, 5,500 mg, 6,000 mg, 6,500 mg, 7,000 mg, 7,500 mg, 8,000 mg, 8,500 mg, 9,000 mg, 9,500 mg, 10,000 mg, or more of one or more active ingredients.

In some instances, a unit dosage contains at least about 1 to about 2,000 mg (e.g., about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1,050 mg, 1,075 mg, 1,100 mg, 1,125 mg, 1,150 mg, 1,175 mg, 1,200 mg, 1,225 mg, 1,250 mg, 1,275 mg, 1,300 mg, 1,325 mg, 1,350 mg, 1,375 mg, 1,400 mg, 1,425 mg, 1,450 mg, 1,475 mg, 1,500 mg, 1,525 mg, 1,550 mg, 1,575 mg, 1,600 mg, 1,625 mg, 1,650 mg, 1,675 mg, 1,700 mg, 1,725 mg, 1,750 mg, 1,775 mg, 1,800 mg, 1,825 mg, 1,850 mg, 1,875 mg, 1,900 mg, 1,925 mg, 1,950 mg, 1,975 mg, or 2,000 mg, or more) of niclosamide analog(s). Multiple doses may be given one or more times per day (e.g., 1, 2, 3, 4, 5, 6, 7, 8, 9, or 10 doses per day), or one or more times per week (e.g., 1, 2, 3, 4, 5, 6, or 7) times per week, with one or more doses being given per day.

In some instances, a unit dosage contains at least about 1 to about 2,000 mg (e.g., about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1,050 mg, 1,075 mg, 1,100 mg, 1,125 mg, 1,150 mg, 1,175 mg, 1,200 mg, 1,225 mg, 1,250 mg, 1,275 mg, 1,300 mg, 1,325 mg, 1,350 mg, 1,375 mg, 1,400 mg, 1,425 mg, 1,450 mg, 1,475 mg, 1,500 mg, 1,525 mg, 1,550 mg, 1,575 mg, 1,600 mg, 1,625 mg, 1,650 mg, 1,675 mg, 1,700 mg, 1,725 mg, 1,750 mg, 1,775 mg, 1,800 mg, 1,825 mg, 1,850 mg, 1,875 mg, 1,900 mg, 1,925 mg, 1,950 mg, 1,975 mg, or 2,000 mg, or more) of antiandrogen drug(s). Multiple doses may be given one or more times per day (e.g., 1, 2, 3, 4, 5, 6, 7, 8, 9, or 10 doses per day), or one or more times per week (e.g., 1, 2, 3, 4, 5, 6, or 7) times per week, with one or more doses being given per day.

The data obtained from, for example, animal studies (e.g. rodents and monkeys) can be used to formulate a dosage range for use in humans. The dosage of compounds of the present invention lies preferably within a range of circulating concentrations that include the ED₅₀ with little or no toxicity. The dosage can vary within this range depending upon the dosage form employed and the route of administration. For any composition for use in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

It is furthermore understood that appropriate doses of a composition depend upon the potency of the composition with respect to the desired effect to be achieved. When one or more of these compositions is to be administered to a mammal, a physician, veterinarian, or researcher may, for example, prescribe a relatively low dose at first, subsequently increasing the dose until an appropriate response is obtained. In addition, it is understood that the specific dose level for any particular mammal subject will depend upon a variety of factors including the activity of the specific composition employed, the age, body weight, general health, gender, and diet of the subject, the time of administration, the route of administration, the rate of excretion, any drug combination, and the degree of expression or activity to be modulated.

In some embodiments, both the niclosamide analog(s) and antiandrogen drug(s) are administered at the same time. In other embodiments, the niclosamide analog(s) and antiandrogen drug(s) are not administered at the same time but are administered the same number of times per day, or same number of times per week, or some number of times per month (e.g., both are adminsitered once per day, twice per day, once per week, twice per week, and so on). In some other embodiments, the niclosamide analog(s) and antiandrogen drug(s) are given on different dosing schedules. As a non-limiting example, the niclosamide analog(s) are administered once per day, and the antiandrogen drug(s) are adminsitered twice per day, or vice versa. As another non-limiting example, the niclosamide analog(s) are administered once per day, and the antiandrogen drug(s) are administered once every 2, 3, 4, 5, 6, or more days, or vice versa. The skilled artisan will also appreciate that certain factors may influence the dosage and timing required to effectively treat a subject, including but not limited to the severity of the disease or malignant condition, previous treatments, the general health and/or age of the subject, and other diseases present. Moreover, treatment of a subject with a therapeutically effective amount of a composition can include a single treatment or, preferably, can include a series of treatments.

Optimum dosages, toxicity, and therapeutic efficacy of the compositions of the present invention may vary depending on the relative potency of the administered composition and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED50. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

Optimal dosing schedules can be calculated from measurements of active ingredient accumulation in the body of a subject. In general, dosage is from about 1 ng to about 1,000 mg per kg of body weight and may be given once or more daily, weekly, monthly, or yearly. Persons of ordinary skill in the art can easily determine optimum dosages, dosing methodologies and repetition rates. One of skill in the art will be able to determine optimal dosing for administration of a combination of niclosamide analog(s) and antiandrogen drug(s) to a human being following established protocols known in the art and the disclosure herein.

In some embodiments of the present invention, a pharmaceutical composition or medicament is administered to a patient at a therapeutically effective dose to prevent, treat, or control cancer (e.g., prostate cancer, breast cancer, androgen-independent cancer, drug-resistant cancer). The pharmaceutical composition or medicament is administered to a patient in an amount sufficient to elicit an effective therapeutic or diagnostic response in the patient. An effective therapeutic or diagnostic response is a response that at least partially arrests or slows the symptoms or complications of cancer. An amount adequate to accomplish this is defined as “therapeutically effective dose.”

Following successful treatment, it may be desirable to have the subject undergo maintenance therapy to prevent the recurrence of the cancer (e.g., prostate cancer, breast cancer, androgen-independent cancer, drug-resistant cancer).

Determination of an effective amount is well within the capability of those skilled in the art, especially in light of the detailed disclosure provided herein. Generally, an efficacious or effective amount of a composition is determined by first administering a low dose or small amount of the composition, and then incrementally increasing the administered dose or dosages, until a desired effect of is observed in the treated subject with minimal or no toxic side effects.

Single or multiple administrations of the compositions are administered depending on the dosage and frequency as required and tolerated by the patient. In any event, a sufficient quantity of the compositions of this invention to effectively treat the patient should be provided. Generally, the dose is sufficient to prevent, treat, or ameliorate symptoms or signs of disease without producing unacceptable toxicity to the patient.

F. Kits

In another aspect, the present invention provides a kit for preventing or treating cancer in a subject. The kits are useful for treating any cancer, some non-limiting examples of which include prostate cancer, breast cancer, uterine cancer, ovarian cancer, colorectal cancer, stomach cancer, pancreatic cancer, lung cancer (e.g., mesothelioma, lung adenocarcinoma), esophageal cancer, head and neck cancer, sarcomas, melanomas, thyroid carcinoma, CNS cancers (e.g., neuroblastoma, glioblastoma), chronic lymphocytic leukemia, and any other cancer described herein. The kits are also suitable for treating androgen-independent, castrate-resistant, castration recurrent, hormone-resistant, drug-resistant, and metastatic castrate-resistant cancers.

In some embodiments, the kits comprise a niclosamide analog and an antiandrogen drug. In some other embodiments, the kits further comprise a pharmaceutically acceptable carrier. In particular embodiments, the niclosamide analog is a compound according to Formula (I):

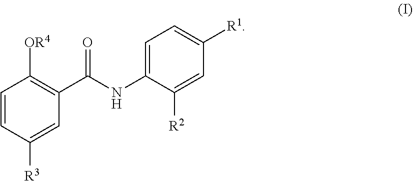

In some embodiments, R¹ is selected from the group consisting of X, CX₃, NO₂, OH, and alkoxy; R² is selected from the group consisting of H, X, CX₃, NO₂, OH, and alkoxy; R³ is selected from the group consisting of X, CX₃, NO₂, OH, and alkoxy; and R⁴ is selected from the group consisting of H and C(O)R⁵, wherein R⁵ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R¹ is CX₃ or NO₂. In other instances, R² is H or X. In some other instances, R³ is X. In other instances, R⁵ is C₂ alkyl or C₂ alkenyl. In particular instances, X is independently selected from the group consisting of F and Cl.

In some embodiments, R¹ is CX₃ (e.g., CF₃) and R² is H or X (e.g., Cl). In some embodiments, R¹ is CX₃, R³ is X (e.g., Cl), and R⁴ is H. In some embodiments, R¹ is CX₃ (e.g., CF₃), R² is H or X (e.g., Cl), R³ is X (e.g., Cl), and R⁴ is H. In some embodiments, R¹ is NO₂ and R⁴ is

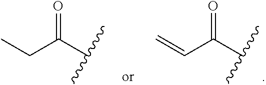

In some embodiments, R² is X (e.g., Cl) and R⁴ is

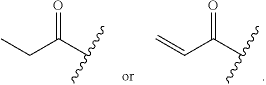

In some embodiments, R³ is X (e.g., Cl) and R⁴ is

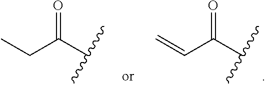

In some embodiments, R¹ is NO₂, R² is X (e.g., Cl), R³ is X (e.g., Cl), and R⁴ is

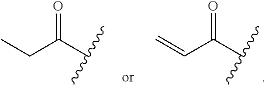

In some embodiments, R¹ is not NO₂ when R² is Cl, R³, is Cl, and R⁴ is H. In some embodiments, R² is not Cl when R¹ is NO₂, R³ is Cl, and R⁴ is H. In some embodiments, R³ is not Cl when R¹ is NO₂, R² is Cl, and R⁴ is H. In some embodiments, R⁴ is not H when R¹ is NO₂, R² is Cl, and R³ is Cl.

In some embodiments, the compound of Formula (I) is selected from the group consisting of

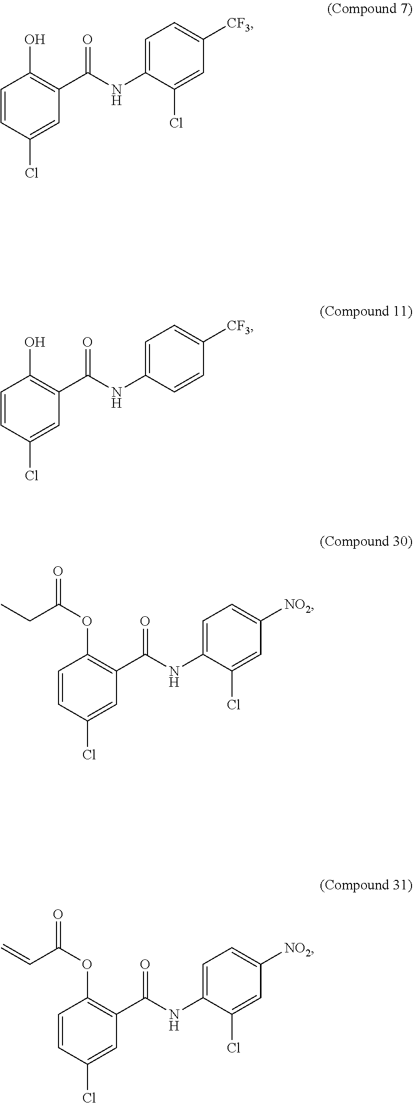 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.

In some embodiments, the niclosamide analog is a compound according to Formula (II):

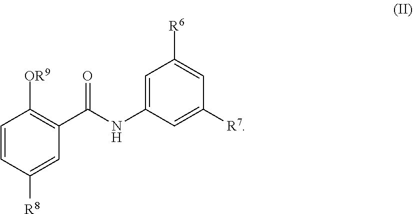

In some embodiments, R⁶ and R⁷ are independently selected from the group consisting of H, X, CX₃, NO₂, OH, and alkoxy; R⁸ is selected from the group consisting of X, CX₃, NO₂, OH, and alkoxy; and R⁹ is selected from the group consisting of H and C(O)R¹⁰, wherein R¹⁰ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen. In some instances, R⁶ and/or R⁷ are CX₃. In some other instances, R⁸ is X. In some instances, R⁹ is H. In particular instances, X is independently selected from the group consisting of F and Cl.

In some embodiments, R⁶ and R⁷ are CX₃ (e.g., CF₃). In some embodiments, R⁶ and R⁷ are CX₃ (e.g., CF₃) and R⁸ is X (e.g., Cl). In some embodiments, R⁶ and R⁷ are CX₃ (e.g., CF₃) and R⁹ is H. In some embodiments, the compound of Formula (II) is

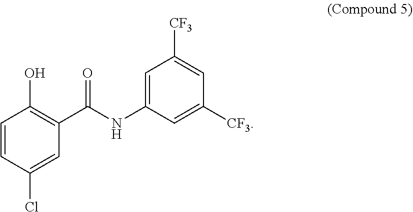 [SMILES: O=C(Nc1cc(C(F)(F)F)cc(C(F)(F)F)c1)c1cc(Cl)ccc1O]

In some embodiments, R⁶ is not F when R⁷ is F, R⁸ is Cl, and R⁹ is H. In some embodiments, R⁷ is not F when R⁶ is F, R⁸ is Cl, and R⁹ is H.

In some embodiments, the compound of Formula (I) or (II) is not niclosamide or Compound 1, 2, 8, 17, 29, 34, or 35.

In some embodiments, the niclosamide analog is a compound of Formula (I) and a compound of Formula (II).

In some embodiments, the antiandrogen drug is a non-steroidal androgen receptor antagonist, a CYP17A1 inhibitor, or a combination thereof. Suitable non-steroidal AR antagonists include bicalutamide (Casodex, Cosudex, Calutide, Kalumid), flutamide, nilutamide, apalutamide (ARN-509, JNJ-56021927), darolutamide, enzalutamide (Xtandi), cimetidine and topilutamide. Suitable CYP17A1 inhibitors include abiraterone acetate (Zytiga), ketoconazole, and seviteronel. Any combination of antiandrogen drugs can be used in kits of the present invention.

Materials and reagents to carry out the various methods of the present invention can be provided in kits to facilitate execution of the methods. As used herein, the term “kit” includes a combination of articles that facilitates a process, assay, analysis, or manipulation. In particular, kits of the present invention find utility in a wide range of applications including, for example, diagnostics, prognostics, therapy, and the like.

Kits can contain chemical reagents as well as other components. In addition, the kits of the present invention can include, without limitation, instructions to the kit user, apparatus and reagents for sample collection and/or purification, apparatus and reagents for product collection and/or purification, apparatus and reagents for administering niclosamide analog(s) and/or antiandrogen drug(s), apparatus and reagents for determining the level(s) of biomarker(s), sample tubes, holders, trays, racks, dishes, plates, solutions, buffers or other chemical reagents, suitable samples to be used for standardization, normalization, and/or control samples. Kits of the present invention can also be packaged for convenient storage and safe shipping, for example, in a box having a lid.

In some embodiments, the kits also contain negative and positive control samples for detection of biomarkers. Non-limiting examples of suitable biomarkers include prostate-specific antigen (PSA), alpha-methylacyl-CoA racemase (AMACR), endoglin (CD105), engrailed 2 (EN-2), prostate-specific membrane antigen (PSMA), caveolin-1, interleukin-6 (IL-6), CD147, members of the S100 protein family (e.g., S100A2, S100A4, S100A8, S100A9, S100A11), annexin A3 (ANXA3), human kallikrein-2 (KLK2), TGF-Betal, beta-microseminoprotein (MSMB), estrogen receptor (ER), progesterone receptor (PgR), HER2, Ki67, cyclin D1, and cyclin E. In some instances, the one or more biomarkers comprises PSA. In some embodiments, the negative control samples are obtained from individuals or groups of individuals who do not have cancer. In other embodiments, the positive control samples are obtained from individuals or groups of individuals who have cancer. In some embodiments, the kits contain samples for the preparation of a titrated curve of one or more biomarkers in a sample, to assist in the evaluation of quantified levels of the one or more biomarkers in a test biological sample.

### IV. EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

### Small Molecule Inhibitors of Androgen Receptor Variants

This example describes the identification of several niclosamide analogs that functioned as small molecule androgen receptor (AR) modulators. The compounds significantly inhibited AR-V7 expression and suppressed enzalutamide/abiraterone resistant tumor growth and induced apoptosis of resistant prostate cancer cells in vitro and in vivo. In addition, some compounds blocked the activity of mutant ARs, including the K581R, L702H, T878A, and V716M variants that confer resistance to antiandrogen therapies such as enzalutamide. Furthermore, some compounds were able to synergize with enzalutamide, abiraterone, bicalutamide and improve their therapeutic activity.

### Introduction

Prostate cancer is the second leading cause of cancer-related death and the most commonly diagnosed cancer in men, with an estimated 220,800 new cases annually in the United States alone (1,2). First-line treatments for prostate cancer aim to reduce circulating androgen levels through the use of androgen deprivation therapies (ADT). This is accomplished using one of two methods: surgical bilateral orchiectomy, which inhibits androgen synthesis by the testes, or the use of castration-inducing drugs to reduce androgen levels and androgen receptor (AR) activation. While ADT is initially effective at reducing prostate cancer growth, after two to three years of treatment the majority of patients progress to castration-resistant prostate cancer (CRPC) and tumor growth will proceed even in the presence of castrate levels of androgen. At this point of disease progression, the number of therapeutic options is currently limited but is the focus of intense research to improve the outcome for patients (3).

Clinically, CRPC is defined as the progression of prostate cancer in the presence of castrate levels of circulating testosterone (4,5). Often, the AR is either overexpressed, hyper-activated, or both, leading to the transcription of downstream target genes which ultimately promote tumor progression despite the patient having negligible levels of androgen present. The mechanisms which lead to the development of CRPC from hormone-sensitive prostate cancer are widely studied. The identified mechanisms include AR amplification and mutation, AR co-activator and co-repressor modifications, aberrant activation and/or post-translational modification, AR splice variants, and altered steroidogenesis, each of which results in an increase in AR activation and signaling. This can be due to an increased amount of androgen, enhanced response to existing androgen, and activation of the AR by non-classical ligands or no ligand at all, among other methods (6-10).

Treatment of CRPC is currently achieved with the administration of taxanes, such as docetaxel and cabazitaxel, which interrupt the growth of fast-dividing cells through disruption of microtubule function, or with next-generation antiandrogen therapies including enzalutamide and abiraterone. The primary mechanism of antiandrogens is to inhibit AR activation either directly, by antagonizing the receptor, or indirectly by blocking androgen synthesis. Unfortunately, it is estimated that one-third of patients given abiraterone and one-fourth of patients given enzalutamide fail to respond to initial treatment with these drugs (11,12). Furthermore, within 12-24 months of initiating treatment, even those who initially respond to the drugs often develop resistance.

Development of resistance to antiandrogen drugs such as enzalutamide and abiraterone is practically inevitable given the several potential pathways of resistance (13,14). Recent studies have linked AR alternative splicing, particularly the splice variant AR-V7, to the development of enzalutamide/abiraterone resistance (15-18). AR splice variants can be formed by genome rearrangement and alternative splicing involving splicing factors such as hnRNPAs (19,20). Most commonly, AR variants lack the C-terminal ligand-binding domain, and these truncated versions of AR are often ligand-independent and result in constitutive activation and uncontrolled downstream AR signaling (21-25). Expression of these AR variants is strongly associated with resistance to both abiraterone and enzalutamide, and though not as well studied, to docetaxel resistance as well. Of the variants, AR-V7, appears to be of particular importance. It has been shown that AR-V7 expression in patients treated with enzalutamide or abiraterone correlates to a significantly lower PSA response, shorter progression-free time, and lower overall survival compared to men who do not express AR-V7 (26). Targeting of AR signaling, especially AR variants, would improve current antiandrogen therapies for advanced prostate cancer.

### Synthesis of Small Molecule Modulators: Niclosamide Analogs

Previously it has been identified that niclosamide, an anthelmintic agent approved by the FDA for the treatment of tapeworm infections, inhibits AR variants such as AR-V7 expression and overcomes resistance to enzalutamide and abiraterone (27). In this work, several analogs of niclosamide were synthesized in order to identify more effective inhibitors of AR variants for the treatment of advanced prostate cancer.

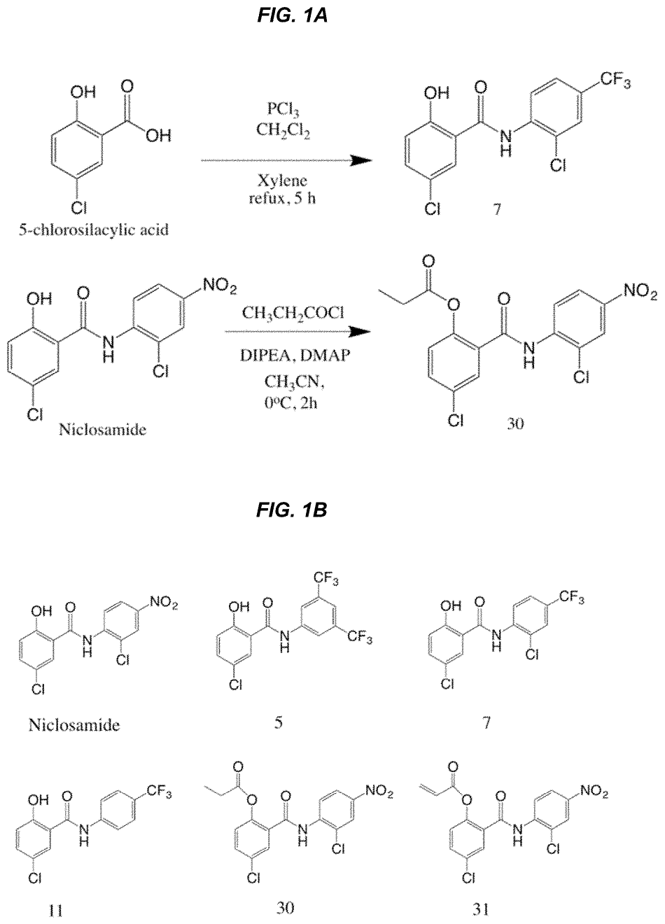

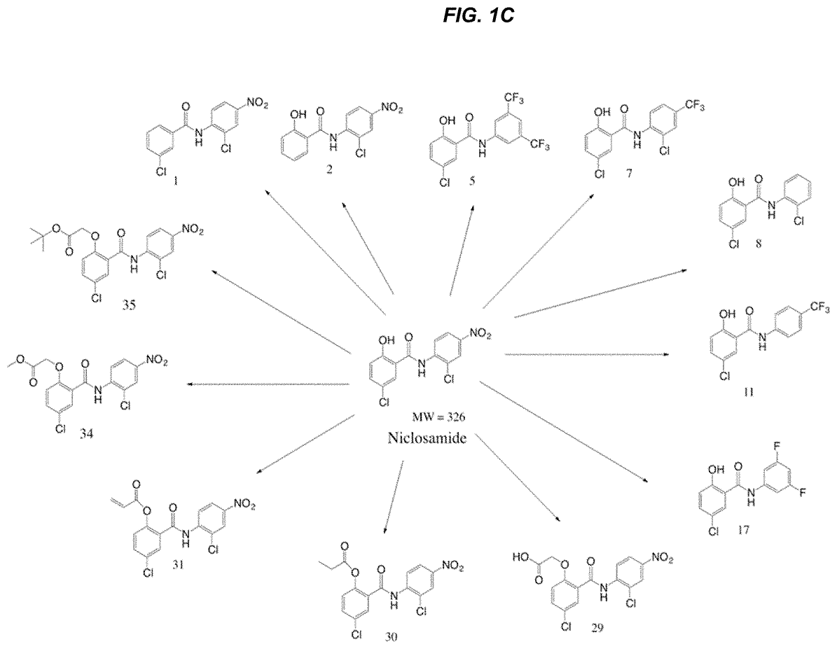

The synthesis of Compounds 7 and 30 is shown in FIG. 1A. Both compounds were synthesized using a one-step reaction. For the synthesis of Compound 7, commercially-available 5-chlorosalicylic acid was coupled with 2-chloro-4-trifluoromethyl aniline using a 2 M solution of phosphorus trichloride in dichloromethane. Xylene was used as the solvent for the reaction and the reaction mixture was refluxed for 5 hours (or until the reactants disappeared). The hot solution was transferred and brought to room temperature for the product to precipitate out of the solution. The precipitate was then re-dissolved in ethyl acetate and recrystallized to obtain the pure Compound 7.

For the synthesis of Compound 30, niclosamide was dissolved in anhydrous CH₃CN. DIPEA (N,N-Diisopropylethylamine) and DMAP (4-Dimethylaminopyridine) were added at −10 ° C. After stirring for 30 minutes, propionyl chloride was added and the reaction mixture was stirred for 2 hours at 0 ° C. Water was then added to the reaction mixture and the mixture was extracted with ethyl acetate. The organic phase was then combined and washed successively with water and brine, dried with Na₂SO_{4,} then evaporated under reduced pressure to afford a light yellow solid. The compound was purified with column chromatography to afford Compound 30. Compounds 5 and 11 were synthesized with the same procedure as Compound 7, and Compound 31 was obtained in a similar manner as Compound 30. The chemical structures of the newly synthesized compounds are illustrated in FIGS. 1B and 1C.

### Identification of Small Molecule Modulators as Potent Inhibitors of AR Variants

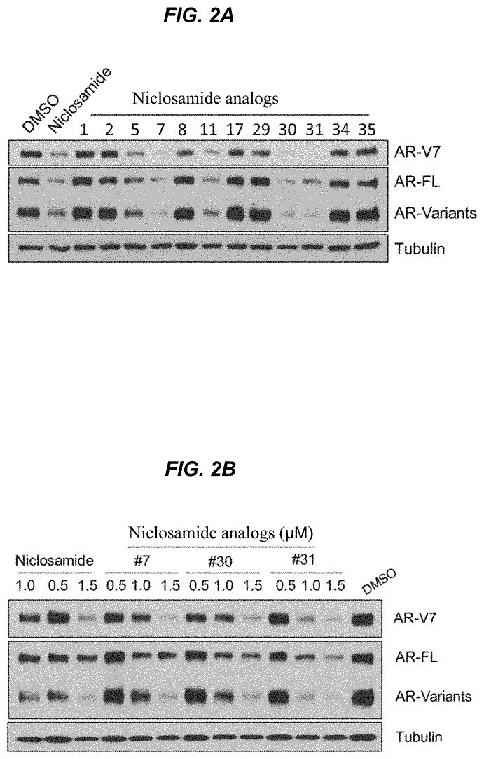

The effects of the synthetic small molecule modulators on AR and AR variant expression were determined. CWR22Ry1 prostate cancer cells were treated with different compounds. Protein lysates were analyzed for AR and AR variant expression. FIG. 2A shows that Compounds 5, 7, 11, 30, and 31 inhibited AR and AR-V7 expression. In contrast, Compounds 1, 2, 8, 17, 29, 34, and 35 showed no effect on the levels of expression of the AR and AR variants. Further studies showed that Compounds 7, 30, and 31 inhibited AR and AR-V7 expression in a dose-dependent manner (FIG. 2B).

### Small Molecule Modulators Inhibited AR-V7-Mediated Transcriptional Activity

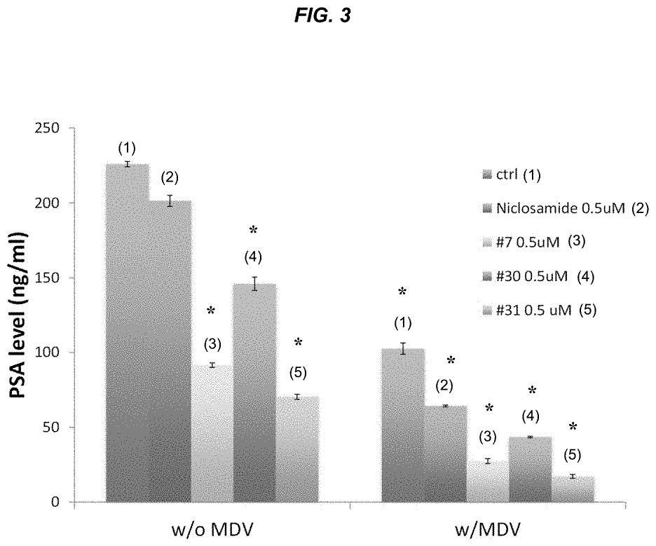

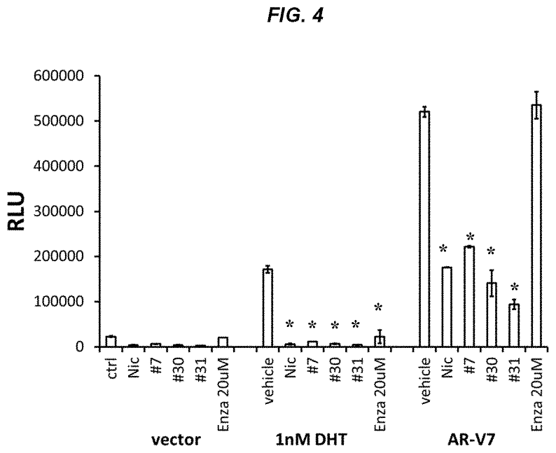

To determine the effects of these compounds on AR-V7 transcriptional activity, the levels of PSA protein expression were measured in C4-2 prostate cancer cells that overexpress AR-V7 (C4-2-V7) that were treated with niclosamide analogs. As shown in FIG. 3, Compounds 7, 30, and 31 inhibited PSA expression, with Compound 31 showing the strongest effect. Furthermore, Compounds 7, 30, and 31 further enhanced enzalutamide-mediated PSA inhibition (FIG. 3). In addition, the effects on AR transcriptional activity were determined using PSA-luc as a reporter. LNCaP cells were cotransfected with PSA-luc together with or without AR-V7, and treated with the compounds. As shown in FIG. 4, enzalutamide and various niclosamide analogs inhibited androgen (DHT)-induced PSA-luc activity in the absence of AR-V7. However, only Compounds 7, 30, and 31 but not enzalutamide inhibited AR-V7 mediated PSA-luc activity. These data show that Compounds 7, 30, and 31 but not enzalutamide inhibited AR-V7-mediated transcriptional activity.

### Small Molecule Modulators Blocked Mutant AR Transcriptional Activity

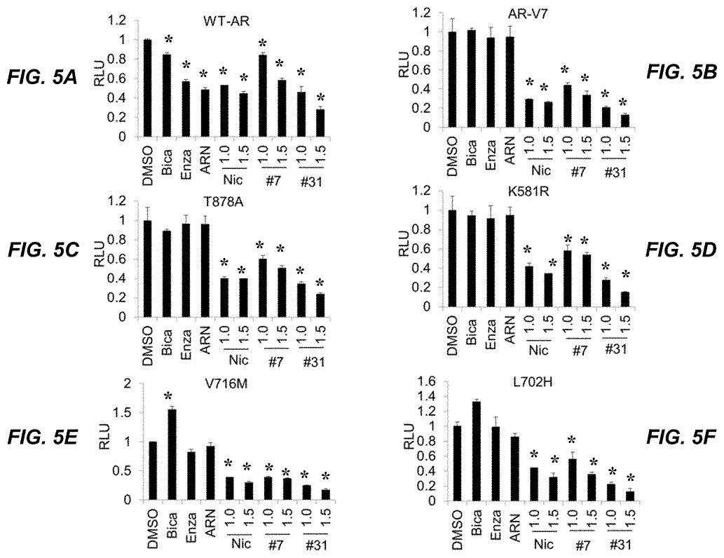

The emergence of mutations in AR has been shown to drive resistance to antiandrogen therapies. The effects of antiandrogens and niclosamide analogs on mutant ARs were studied in transactivation assays in HEK 293 cells that were transiently transfected with expression vectors encoding the corresponding mutant AR and an androgen-responsive luciferase reporter gene construct. Bicalutamide (Bica), enzalutamide (Enza), ARN509 (ARN), niclosamide (Nic), and Compounds 7 and 31 blocked wild-type AR transactivation. The T878A, K581R, L702H and V716M AR mutations switched bicalutamide from antagonist to agonist, whereas enzalutamide and ARN509 had no effect in blocking the transcriptional activity of these mutant ARs. However, niclosamide and Compounds 7 and 31 significantly blocked the transactivation of all tested mutant ARs and AR-V7 (FIG. 5).

Niclosamide Analogs Inhibited Prostate Cancer Cell Growth and Induced Cell Apoptosis in vitro

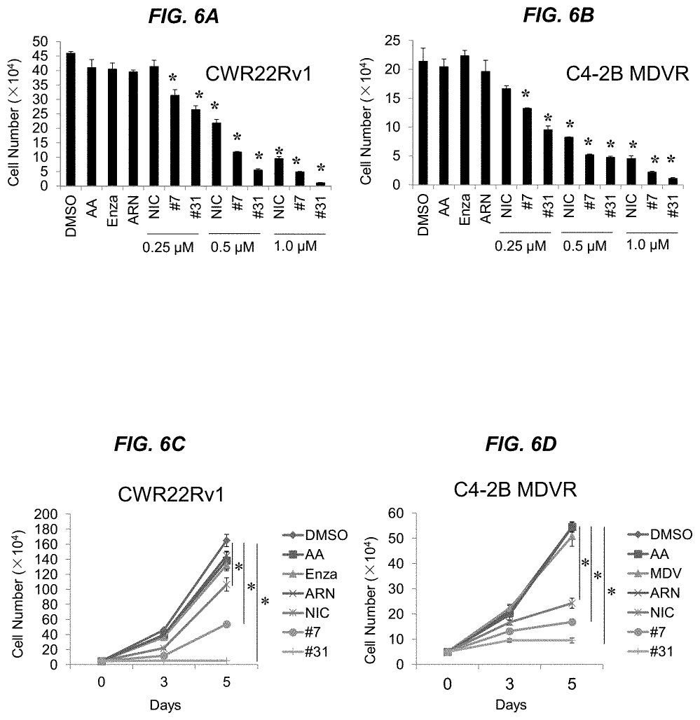

To examine the effects of these identified small molecular modulators on the growth of resistant prostate cancer cells, CWR22Rv1 and C4-2B MDVR cells were treated with either abiraterone, enzalutamide, ARN509, or increasing doses of niclosamide, Compound 7, or Compound 31 for 48 hours, after which time cell numbers were counted. As shown in FIG. 6, abiraterone (AA), enzalutamide (Enza) and ARN509 (ARN) did not inhibit the cell growth in either cell line. However, niclosamide (NIC) and Compounds 7 and 31 were able to inhibit cell growth in a dose-dependent manner. The bottom panels show the time-dependent growth effects of abiraterone, enzalutamide, ARN509, niclosamide, and Compounds 7 and 31.

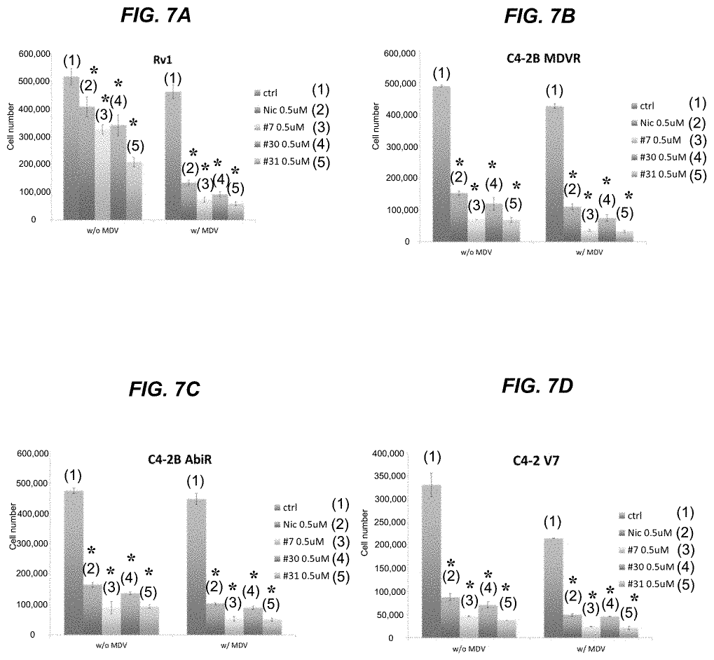

To further examine whether these compounds affected prostate cancer cell growth, CWR22Rv1, C4-2B MDVR, C4-2B AbiR, and C4-2-V7 cells were treated with DMSO or 0.5 μM niclosamide or one of the niclosamide analogs for 48 hours, after which time cell numbers were determined. As shown in FIG. 7, 0.5 μM niclosamide and niclosamide analogs significantly inhibited cell growth in prostate cancer cells. In addition, a combination of enzalutamide (MDV) with either niclosamide or the analogs Compounds 7, 30, and 31 further inhibited cell proliferation. To further examine the anti-cancer effects of these compounds, an ELISA cell death assay was performed. As shown in FIG. 8, niclosamide significantly induced cell apoptosis in prostate cancer cells, and Compounds 7, 30, and 31 further enhanced the induction of apoptosis. A combination of enzalutamide (MDV) with these compounds further increased apoptotic cell death. Collectively, these results show that Compounds 7, 30, and 31 inhibited prostate cancer cell growth and induced cell apoptosis, and further enhanced the activity of enzalutamide in resistant prostate cancer cells.

Identification of Niclosamide Analogs that Synergized with Antiandrogen Drugs

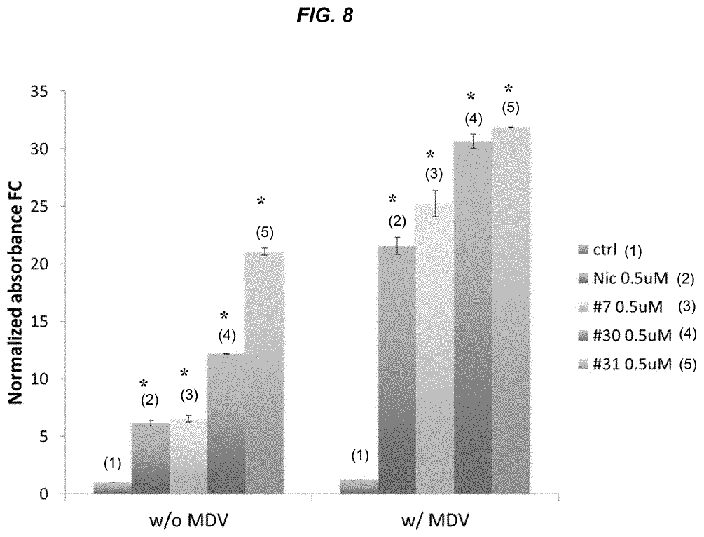

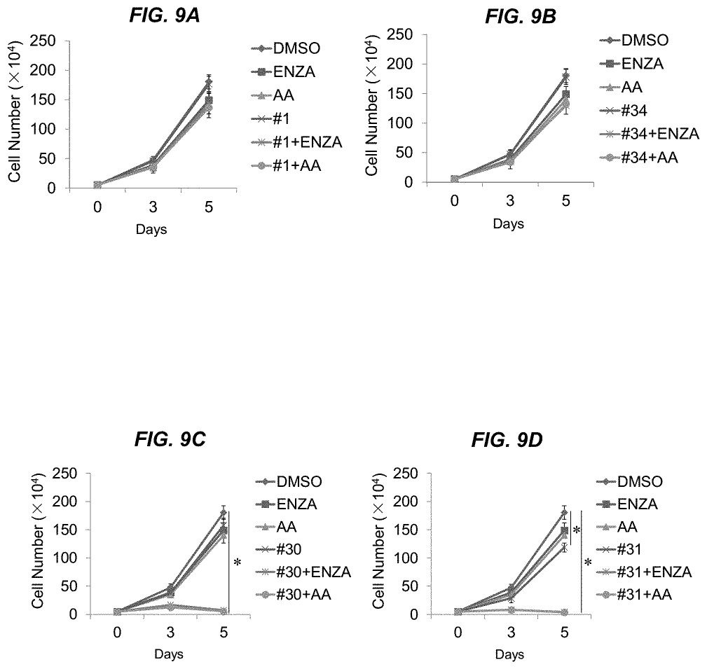

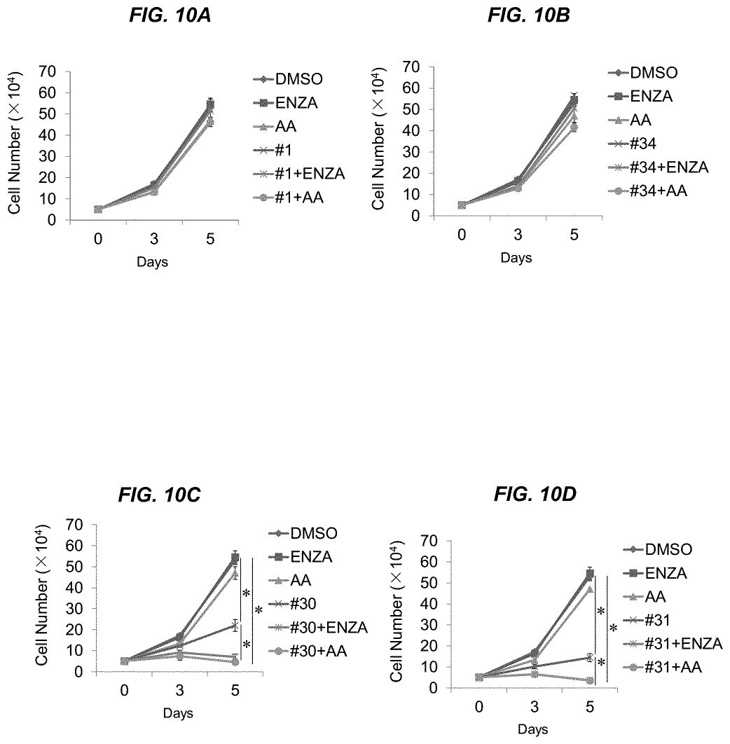

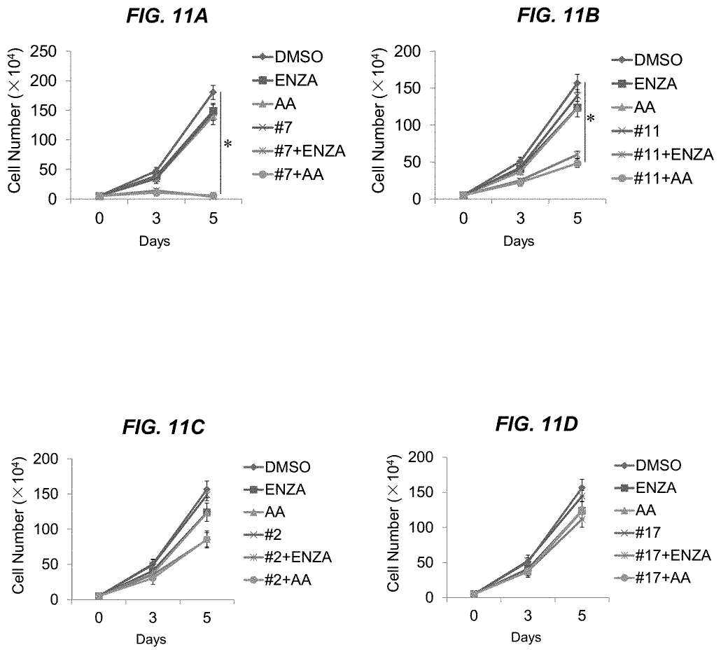

Since AR-V7 is critically involved in driving resistance to antiandrogens such as enzalutamide and abiraterone, it was hypothesized that the compounds with the ability to inhibit AR-V7 would synergize with antiandrogens and improve their therapeutic activity in resistant prostate cancer. To test this hypothesis, the effects of niclosamide analogs on cell growth in combination with antiandrogens such as enzalutamide and abiraterone were determined. Compounds 30 and 31 (both of which inhibit AR-V7 expression) were compared to Compounds 1 and 34 (neither of which inhibit AR-V7 expression) for their effect on the growth of resistant prostate cancer cells. As shown in FIG. 9, CWR22Ry1 cells were resistant to both enzalutamide and abiraterone. Neither of Compounds 1 or 34 alone were able to inhibit the cellular growth of CWR22Ry1 cells, nor the was the combination of these compounds with enzalutamide or abiraterone able to inhibit the growth of CWR22Ry1 cells. In contrast, a combination of either Compound 30 or 31 with enzalutamide or abiraterone significantly inhibited the growth of CWR22Ry1 cells (FIG. 9). Similar results were observed with enzalutamide in resistant C4-2BMDVR cells, in which Compounds 30 and 31 but neither Compound 1 nor Compound 34 were able to synergize with enzalutamide or abiraterone (FIG. 10). These results show that Compounds 30 and 31 can be specifically utilized to combine with antiandrogen drugs (e.g., enzalutamide, abiraterone) to overcome resistance to antiandrogen therapy. Compounds 7 and 11 were also tested (both of which inhibit AR-V7 expression) versus Compounds 2 and 17 (neither of which inhibit AR-V7 expression) in combination with either enzalutamide or abiraterone in enzalutamide/abiraterone resistant CWR22Ry1 cells. As shown in FIG. 11, neither Compounds 2 nor Compound 17 alone were able to inhibit the growth of CWR22Rv1 cells, nor was a combination of either of these compounds with enzalutamide or abiraterone able to inhibit the growth of CWR22Rv1 cells. In contrast, combinations of either Compound 7 or 11 with enzalutamide or abiraterone were able to significantly inhibit the growth of CWR22Rv1 cells. Collectively, it was demonstrated that compounds that had the ability to inhibit AR-V7 expression (e.g., Compounds 7, 11, 30, and 31) could synergize with enzalutamide or abiraterone, while compounds that failed to inhibit AR-V7 expression (e.g., Compounds 1, 2, 17, and 34) were not able to synergize with either enzalutamide or abiraterone.

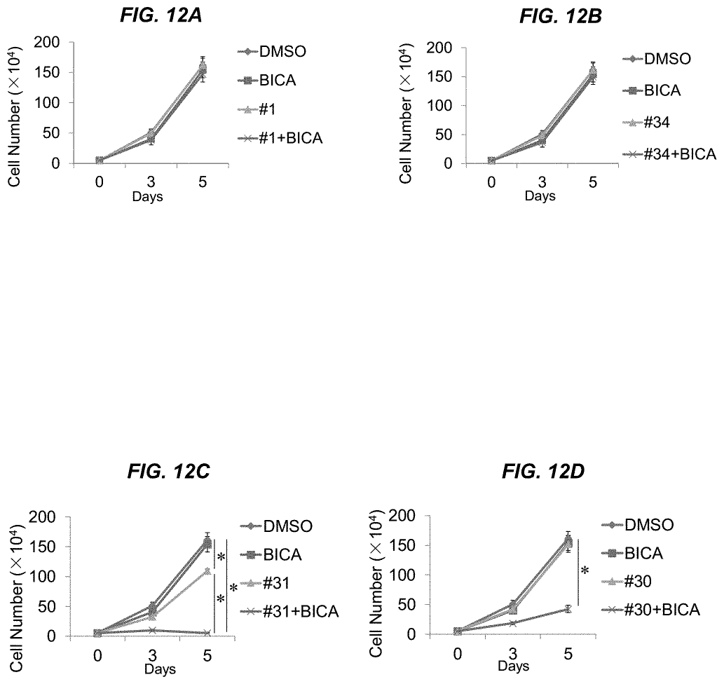

Next it was determined whether the selective compounds that were able to synergize with enzalutamide and abiraterone could also synergize with bicalutamide. None of Compounds 1, 7, 30, 31, or 34 alone were able to inhibit CWR22Rv1 cell growth (FIG. 12). Combinations of Compounds 1 and 34 with bicalutamide were not able to inhibit the cell growth. However, combinations of Compound 7, 30, or 31 with bicalutamide were able to significantly inhibit the growth of CWR22Rv1 cells (FIG. 12). These results show that selective compounds can be combined with bicalutamide to treat advanced resistant prostate cancer cells.

Niclosamide Analogs Inhibited Prostate Cancer Tumor Growth in vivo

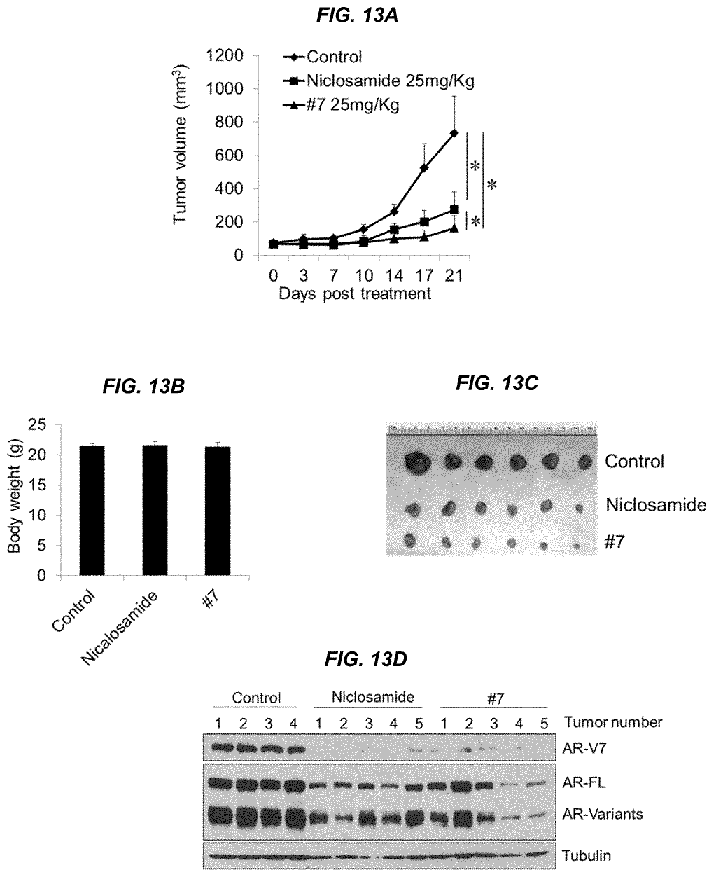

To determine whether niclosamide analogs could inhibit enzalutamide/abiraterone resistant tumor cell growth in vivo, CWR22Rv1 tumor xenografts were treated with Compound 7. As shown in FIGS. 13A-13C, Compound 7 significantly inhibited the tumor growth without inhibition of body weight. Protein lysates were isolated and analyzed for AR and AR-V7 expression by Western blot. As shown in FIG. 13D, both AR and AR-V7 protein expression was significantly inhibited by the treatment with Compound 7 as compared to the controls. These results show that Compound 7 inhibited enzalutamide/abiraterone resistant tumor growth by suppression of AR and AR-V7 expression.

### Summary

These experiments identified several niclosamide analogs that functioned as small molecule modulators of AR and AR-V7 and which inhibited resistant prostate cancer tumor growth and induced apoptosis. Furthermore, Compounds 30 and 31 were identified as being able to synergize with enzalutamide, abiraterone, and bicalutamide and improve their therapeutic activity for advanced prostate cancer.

### REFERENCES

1. Ferlay J, Steliarova-Foucher E, Lortet-Tieulent J, Rosso S, Coebergh J W, Comber H, et al. Cancer incidence and mortality patterns in Europe: estimates for 40 countries in 2012. European journal of cancer 2013;49(6):1374-403.
2. Siegel R L, Miller K D, Jemal A. Cancer statistics, 2015. CA: a cancer journal for clinicians 2015;65(1):5-29.
3. Harris W P, Mostaghel E A, Nelson P S, Montgomery B. Androgen deprivation therapy: progress in understanding mechanisms of resistance and optimizing androgen depletion. Nature clinical practice Urology 2009;6(2):76-85.
4. Cookson M S, Roth B J, Dahm P, Engstrom C, Freedland S J, Hussain M, et al. Castration-resistant prostate cancer: AUA Guideline. The Journal of urology 2013;190(2):429-38.
5. Saad F, Hotte S J. Guidelines for the management of castrate-resistant prostate cancer. Canadian Urological Association journal=Journal de l'Association des urologues du Canada 2010;4(6):380-4.
6. Dehm S M, Schmidt L J, Heemers H V, Vessella R L, Tindall D J. Splicing of a novel androgen receptor exon generates a constitutively active androgen receptor that mediates prostate cancer therapy resistance. Cancer Res 2008;68(13):5469-77.
7. Chang K H, Ercole C E, Sharifi N. Androgen metabolism in prostate cancer: from molecular mechanisms to clinical consequences. British journal of cancer 2014;111(7):1249-54.
8. Chang K H, Li R, Papari-Zareei M, Watumull L, Zhao Y D, Auchus R J, et al. Dihydrotestosterone synthesis bypasses testosterone to drive castration-resistant prostate cancer. Proceedings of the National Academy of Sciences of the United States of America 2011;108(33):13728-33.
9. Shtivelman E, Beer T M, Evans C P. Molecular pathways and targets in prostate cancer. Oncotarget 2014;5(17):7217-59.
10. Steketee K, Timmerman L, Ziel-van der Made A C, Doesburg P, Brinkmann A O, Trapman J. Broadened ligand responsiveness of androgen receptor mutants obtained by random amino acid substitution of H874 and mutation hot spot T877 in prostate cancer. International journal of cancer Journal international du cancer 2002;100(3):309-17.
11. de Bono J S, Logothetis C J, Molina A, Fizazi K, North S, Chu L, et al. Abiraterone and increased survival in metastatic prostate cancer. The New England journal of medicine 2011 ;364(21): 1995-2005.
12. Scher H I, Fizazi K, Saad F, Taplin M E, Sternberg C N, Miller K, et al. Increased survival with enzalutamide in prostate cancer after chemotherapy. The New England journal of medicine 2012;367(13):1187-97.
13. Scher H I, Beer T M, Higano C S, Anand A, Taplin M E, Efstathiou E, et al. Antitumour activity of MDV3100 in castration-resistant prostate cancer: a phase 1-2 study. Lancet 2010;375(9724):1437-46.
14. Kim W, Ryan C J. Androgen receptor directed therapies in castration-resistant metastatic prostate cancer. Curr Treat Options Oncol 2012;13(2):189-200.
15. Nadiminty N, Tummala R, Liu C, Yang J, Lou W, Evans C P, et al. NF-kappaB2/p52 induces resistance to Enzalutamide in Prostate Cancer: Role of androgen receptor and its variants. Molecular cancer therapeutics 2013;12:1629-37.
16. Joseph J D, Lu N, Qian J, Sensintaffar J, Shao G, Brigham D, et al. A clinically relevant androgen receptor mutation confers resistance to 2nd generation antiandrogens enzalutamide and ARN-509. Cancer discovery 2013;3(9):1020-9.
17. Korpal M, Korn J M, Gao X, Rakiec D P, Ruddy D A, Doshi S, et al. An F876L Mutation in Androgen Receptor Confers Genetic and Phenotypic Resistance to MDV3100 (Enzalutamide). Cancer discovery 2013;3(9):1030-43.
18. Nyquist M D, Li Y, Hwang T H, Manlove L S, Vessella R L, Silverstein K A, et al. TALEN-engineered AR gene rearrangements reveal endocrine uncoupling of androgen receptor in prostate cancer. Proc Natl Acad Sci U S A 2013;110(43):17492-7.
19. Nadiminty N, Tummala R, Liu C, Lou W, Evans C P, Gao A C. NF-kappaB2/p52:c-Myc:hnRNPA1 Pathway Regulates Expression of Androgen Receptor Splice Variants and Enzalutamide Sensitivity in Prostate Cancer. Molecular cancer therapeutics 2015;14(8):1884-95.
20. Li Y, Alsagabi M, Fan D, Bova G S, Tewfik A H, Dehm S M. Intragenic rearrangement and altered RNA splicing of the androgen receptor in a cell-based model of prostate cancer progression. Cancer research 2011;71(6):2108-17.
21. Dehm S M, Tindall D J. Alternatively spliced androgen receptor variants. Endocrine-related cancer 2011;18(5):R183-96.
22. Guo Z, Yang X, Sun F, Jiang R, Linn D E, Chen H, et al. A novel androgen receptor splice variant is up-regulated during prostate cancer progression and promotes androgen depletion-resistant growth. Cancer Res 2009;69(6):2305-13.
23. Hu R, Dunn T A, Wei S, Isharwal S, Veltri R W, Humphreys E, et al. Ligand-independent androgen receptor variants derived from splicing of cryptic exons signify hormone-refractory prostate cancer. Cancer Res 2009;69(1):16-22.
24. Sun S, Sprenger C C, Vessella R L, Haugk K, Soriano K, Mostaghel E A, et al. Castration resistance in human prostate cancer is conferred by a frequently occurring androgen receptor splice variant. The Journal of clinical investigation 2010;120(8):2715-30.
25. Yang X, Guo Z, Sun F, Li W, Alfano A, Shimelis H, et al. Novel membrane-associated androgen receptor splice variant potentiates proliferative and survival responses in prostate cancer cells. The Journal of biological chemistry 2011;286(41):36152-60.
26. Antonarakis E S, Lu C, Wang H, Luber B, Nakazawa M, Roeser J C, et al. AR-V7 and resistance to enzalutamide and abiraterone in prostate cancer. N Engl J Med 2014;371(11):1028-38.
27. Liu C, Lou W, Zhu Y, Nadiminty N, Schwartz C T, Evans C P, et al. Niclosamide Inhibits Androgen Receptor Variants Expression and Overcomes Enzalutamide Resistance in Castration-Resistant Prostate Cancer. Clinical Cancer Research 2014;20(12):3198-210.

### Example 2

### Inhibition of Full-Length Androgen Receptor and Androgen Receptor Variants by Niclosamide Analogs and Antiandrogen Drugs

This example describes a series of experiments that were performed to further characterize the ability of niclosamide analogs and antiandrogen drugs to inhibit the expression and activity of full-length androgen receptor (AR-FL) and various androgen receptor (AR) variants (ARVs).

### Small Molecule Modulators Inhibit Full-Length Andro₂en Receptor (AR-FL)-Mediated and Androgen Receptor Variant (ARV)-Mediated Transcriptional Activity

The effects of niclosamide analogs and antiandrogen drugs on the transcriptional activity of AR-FL and several ARVs (i.e., AR-V1, AR-V3, AR-V7, AR-V9, and AR-V12) were evaluated using PSA-luc as a reporter. C4-2B cells were cotransfected with pcDNA, with or without AR-V1, AR-V3, AR-V7, AR-V9 or AR-V12, and with PSA-E/P-luciferase plasmids in FBS condition. Following treatment with DMSO, 10 μM abiraterone (Abi), 20 μM enzalutamide (Enza), 20 μM apalutamide (ARN), 1 μM niclosamide (Nic), 1 μM Compound 7, or 1 μM Compound 31 overnight, the PSA luciferase activity was examined.

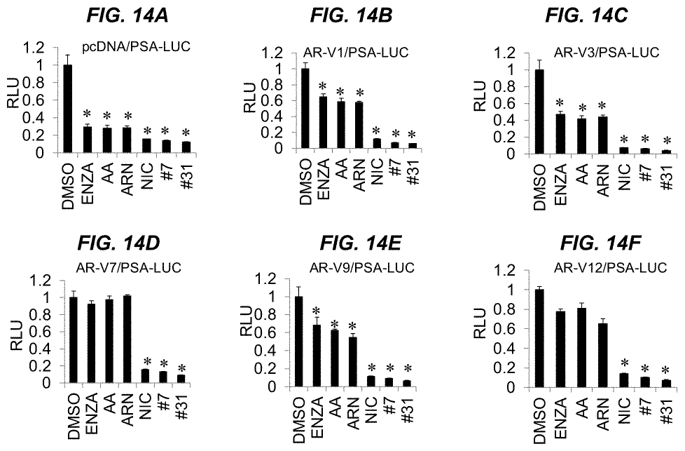

As shown in FIG. 14, niclosamide, Compound 7, and Compound 31 inhibited ARV-mediated PSA-luc activity. In particular, these data show that niclosamide and its analogs inhibited both AR-FL-mediated and ARV-mediated transcriptional activity.

### Small Molecule Modulators Degrade AR-V7 Protein Expression

To determine if the small molecule modulators affect AR-V7 protein expression via protein degradation, CWR22Rv1 and C4-2B MDVR cells were treated with cycloheximide (CHX) and the half-life of AR-V7 protein was measured.

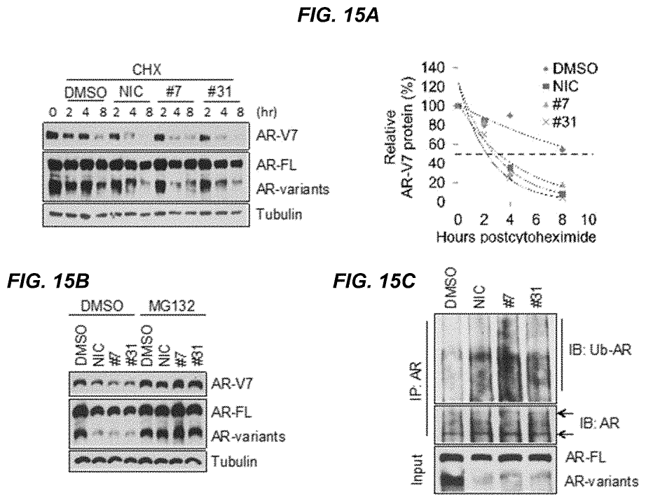

For the data shown in FIG. 15A, CWR22Rv1 cells were treated with 50 μg/mL cycloheximide (CHX) with or without niclosamide (Nic), Compound 7, or Compound 31. After 0, 2, 4, and 8 hours, whole cell lysate was collected and subjected to Western blot. The half-life of AR-V7 was then calculated.

For the data shown in FIG. 15B, CWR22Rv1 cells were treated with niclosamide, Compound 7, or Compound 31 with or without the proteasome inhibitor MG132. Subsequently, the whole cell lysate was collected and subjected to Western blot.

For the data shown in FIG. 15C, CWR22Rv1 cells were treated with Nic, Compound 7, or Compound 31. Subsequently, whole cell lysate was immunoprecipitated with an AR antibody and blotted with ubiquitin and AR antibodies.

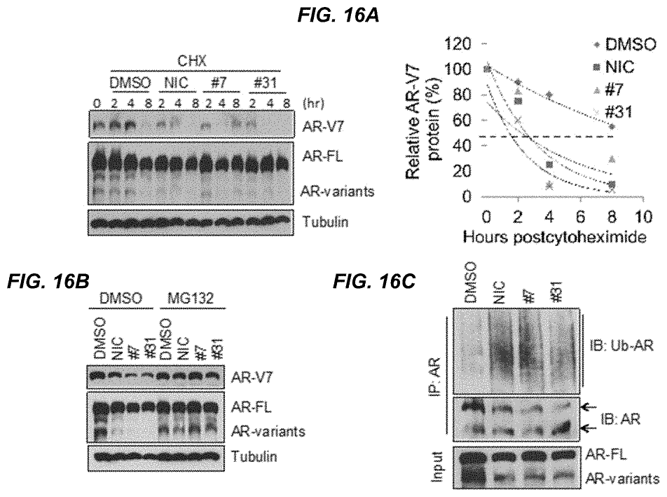

For the data shown in FIG. 16A, C4-2B MDVR cells were treated with 50 μg/mL cycloheximide with or without niclosamide, Compound 7, or Compound 31. After 0, 2, 4, and 8 hours, whole cell lysate was collected and subjected to Western blot. Half-life of AR-V7 was then calculated.

For the data shown in FIG. 16B, C4-2B MDVR cells were treated with niclosamide, Compound 7, or Compound 31, with or without MG132. Whole cell lysate was subsequently collected and subjected to Western blot.

For the data shown in FIG. 16C, C4-2B MDVR cells were treated with niclosamide, Compound 7, or Compound 31. Whole cell lysate was immunoprecipitated with an AR antibody and blot with ubiquitin and AR antibodies.

As shown in FIGS. 15 and 16, niclosamide and its analogs Compound 7 and Compound 31 degraded AR variants via the proteasome-ubiquitination system.

### Small Molecule Modulators Enhance Abiraterone and Apalutamide (ARN509) Treatment in Resistant Prostate Cancer

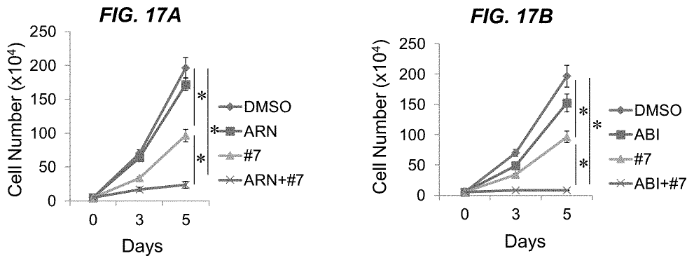

To determine if niclosamide and its analogs enhance abiraterone and apalutamide treatment, CWR22Rv1 cells were treated with a combination of either abiraterone or apalutamide in the presence or absence of Compound 7. For the data shown in FIG. 17A, CWR22Rv1 cells were treated with DMSO, 20 μM apalutamide (ARN), 0.5 μM Compound 7, or a combination of apalutamide and Compound 7. The cell growth was determined at 3,5 days. For the data shown in FIG. 17B, CWR22Rv1 cells were treated with DMSO, 5 μM abiraterone (ABI), 0.5 μM Compound 7, or a combination of abiraterone and Compound 7. The cell growth was determined at 3,5 days.

As shown in FIG. 17, Compound 7 significantly enhanced abiraterone and apalutamide treatment.

### Small Molecule Modulators Showed Better Bioavailability Than Niclosamide in Plasma After Oral Administration in Rats

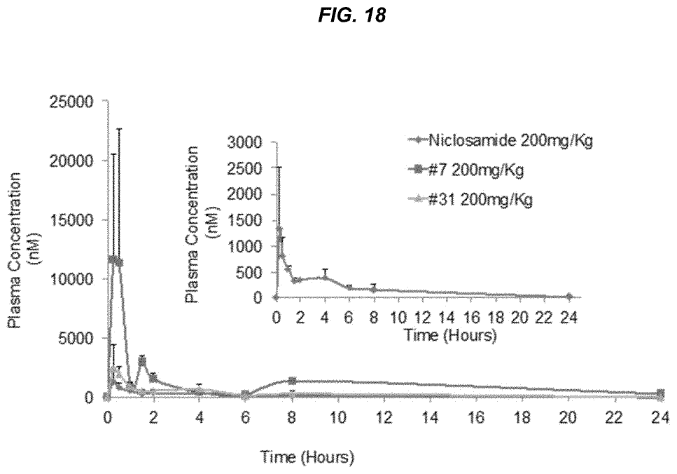

To compare the bioavailability of Compound 7 and Compound 31 to niclosamide, male Sprague-Dawley (SD) rats having body weights of about 300 grams to about 350 grams were administered niclosamide, Compound 7, or Compound 31 orally (p.o.) at a dose of 200 mg/kg. Blood samples were obtained at the time points indicated in FIG. 18 (0 minutes, 15 minutes, 30 minutes, 1 hour, 1.5 hours, 2 hours, 4 hours, 6 hours, 8 hours, and 24 hours) and the plasma was isolated for LC-MS analysis. As shown in FIG. 18, Cmax values for Compound 7 and Compound 31 reached 9038 nM and 2685 nM, respectively, compared to the Cmax of niclosamide of 1556 nM. The results of pharmacokinetic (PK) analysis of Compound 7, Compound 31, and niclosamide are shown in Table 1. These data show that Compound 7 and Compound 31 exhibited much better bioavailability than niclosamide.

TABLE 1
Non-compartment PK analysis of small molecule AR inhibitors in rat plasma

| Pharmacokinetic | Niclosamide | Compound 7 | Compound 31 |
|---|---|---|---|
| parameter | (200 mg/kg) | (200 mg/kg) | (200 mg/kg) |
| HL_Lambda_z (min.) | 328.09 +/− 173.05 | 580.54 +/− 86.4 | 276.51 +/− 92.17 |
| Tₘₐₓ (min.) | 25.00 +/− 8.66 | 22.5 +/− 10.61 | 25 +/− 8.66 |
| Cₘₐₓ (nM) | 1556.93 +/− 974.31 | 9038.31 +/− 8015.51 | 2685.82 +/− 1796.25 |
| AUClast | 248249.95 +/− 91856.79 | 1439633.38 +/− 89486.89 | 382230.2 +/− 220077.03 |
| AUCINF_obs | 1703577.65 +/− 89354.18 | 256881.89 +/− 167532.07 | 388512.79 +/− 217228.83 |
| AUC_% Extrap_obs | 3.67 +/− 3.73 | 15.35 +/− 3.08 | 2.3 +/− 1.85 |

Small Molecule Modulators Inhibit Prostate Cancer Tumor Growth in vivo via Oral Administration

To determine if niclosamide and its analog Compound 7 inhibit tumor growth via oral administration, mice bearing prostate cancer patient-derived xenograft (PDX) model (LuCaP 35CR) tumors were treated with niclosamide or Compound 7 p.o.

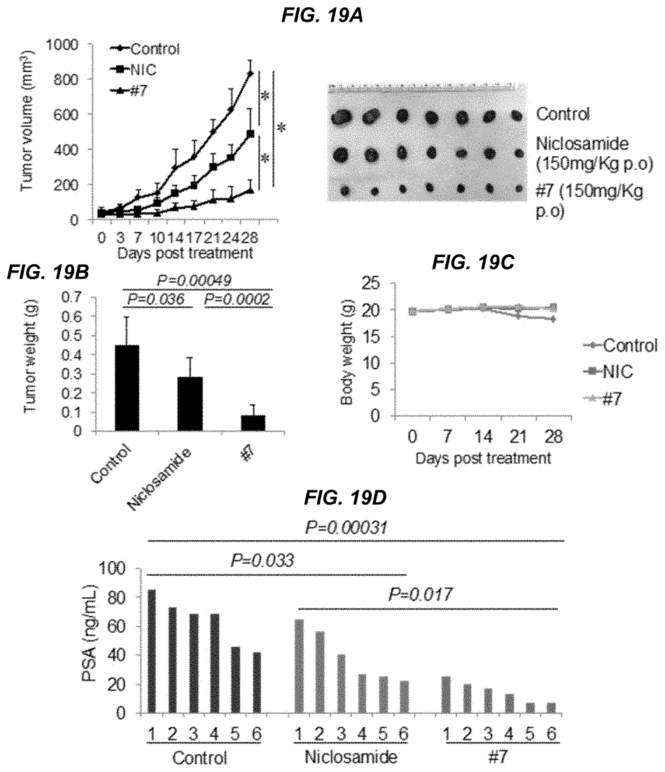

For the data shown in FIG. 19A, a LuCaP 35CR PDX model was established in male castrated SCID mice. When tumor volumes reached around 50-100 mm³, mice were randomly divided to three groups and treated with oral administration of control (0.5% weight/volume (w/v) Methocel A4M), niclosamide (Nic; 150 mg/kg), or Compound 7 (150 mg/kg) for 4 weeks, during which time tumor growth was monitored. Tumor and body weights are shown in FIGS. 19B and 19C, respectively. PSA levels in mouse serum were determined and are shown in FIG. 19D.

As shown in FIG. 19, Compound 7 significantly inhibited tumor growth without inhibition of body weight. PSA analysis in sera obtained from the mice showed that PSA protein expression was inhibited by Compound 7.

### Small Molecule Modulators Inhibit the Growth of Breast Cancer Cells

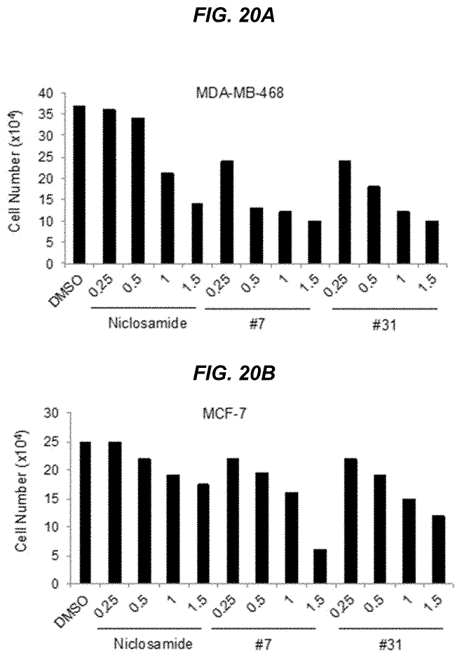

To determine if niclosamide and its analogs Compound 7 and Compound 31 inhibit the growth of breast cancer cells, MDA-MB-468 and MCF-7 cells were treated with niclosamide, Compound 7, or Compound 31 for three days and cell growth was determined. As shown in FIG. 20, both analogs inhibited breast cancer cell growth.

In summary, niclosamide and its analogs Compound 7 and Compound 31 inhibited protein expression of AR-FL and AR variants, as well as AR-FL-mediated and ARV-mediated transcriptional activity. However, the analogs exhibited superior bioavailability compared to niclosamide and were more effective at inhibiting tumor cell growth. In particular, Compound 7 reduced tumor volume in a PDX model and serum PSA levels to a greater extent than niclosamide, and greatly synergized with antiandrogen drugs to inhibit tumor cell growth.

### V. Exemplary Embodiments

Exemplary embodiments provided in accordance with the presently disclosed subject matter include, but are not limited to, the claims and the following embodiments:
1. A composition comprising an antiandrogen drug and a compound according to Formula (I):

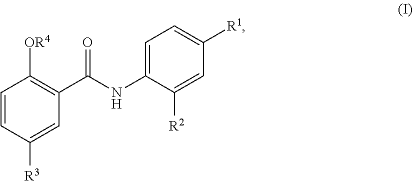

wherein:
R¹ is selected from the group consisting of X, CX₃, NO₂, OH, and alkoxy;
R² is selected from the group consisting of H, X, CX₃, NO₂, OH, and alkoxy;
R³ is selected from the group consisting of X, CX₃, NO₂, OH, and alkoxy; and
R⁴ is selected from the group consisting of H and C(O)R⁵,
wherein R⁵ is selected from the group consisting of H, optionally substituted C₁₋₁₈ alkyl, optionally substituted C₂₋₁₈ alkenyl, and optionally substituted C₂₋₁₈ alkynyl; and
wherein each X is an independently selected halogen.
2. The composition of embodiment 1, wherein R¹ is CX₃ or NO₂.
3. The composition of embodiment 1 or 2, wherein R² is H or X.
4. The composition of any one of embodiments 1 to 3, wherein R³ is X.
5. The composition of any one of embodiments 1 to 4, wherein R⁵ is C₂ alkyl or C₂ alkenyl.
6. The composition of any one of embodiments 1 to 5, wherein X is independently selected from the group consisting of F and Cl.
7. The composition of any one of embodiments 1 to 6, wherein the compound of Formula (I) is selected from the group consisting of

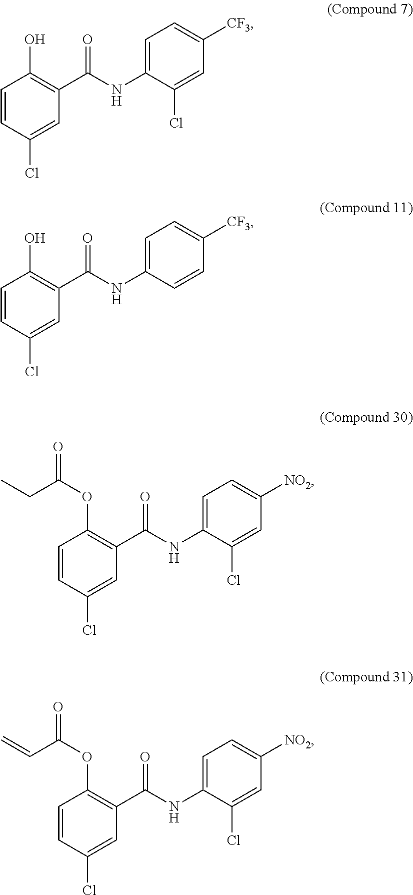 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.
8. The composition of any one of embodiments 1 to 7, wherein the antiandrogen drug is selected from the group consisting of a non-steroidal androgen receptor antagonist, a CYP17A1 inhibitor, and a combination thereof.
9. The composition of embodiment 8, wherein the antiandrogen drug is selected from the group consisting of bicalutamide, apalutamide, enzalutamide, abiraterone acetate, and a combination thereof.
10. The composition of any one of embodiments 1 to 9, wherein the composition inhibits the expression and/or activity of an androgen receptor or a variant thereof.
11. The composition of embodiment 10, wherein the androgen receptor variant is selected from the group consisting of a splice variant, a mutant variant, and a combination thereof.
12 . The composition of embodiment 11, wherein the splice variant is an AR-V1, AR-V3, AR-V7, AR-V9, and/or AR-V12 splice variant.
13. The composition of embodiment 12, wherein the splice variant is an AR-V7 splice variant.
14. The composition of embodiment 11, wherein the mutant variant comprises one or more mutations selected from the group consisting of K581R, L702H, T878A, V716M, and a combination thereof relative to the amino acid sequence set forth in SEQ ID NO: 1.
15. The composition of any one of embodiments 1 to 14, wherein the composition is an effective inhibitor of cancer cell proliferation.
16. The composition of embodiment 15, wherein the cancer cell is a prostate cancer cell or a breast cancer cell.
17. The composition of embodiment 15 or 16, wherein the cancer cell is selected from the group consisting of an androgen-independent cancer cell, a metastatic cancer cell, a castrate-resistant cancer cell, a castration recurrent cancer cell, a hormone-resistant cancer cell, a metastatic castrate-resistant cancer cell, and a combination thereof.
18. The composition of any one of embodiments 1 to 17, further comprising a pharmaceutically acceptable carrier.
19. A method for preventing or treating cancer in a subject, the method comprising administering to the subject a therapeutically effective amount of a composition comprising an antiandrogen drug and a compound according to Formula (I):

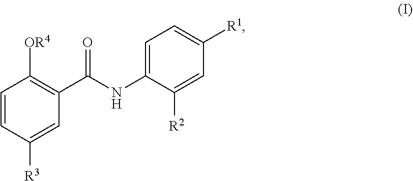

wherein:
R¹ is selected from the group consisting of X, CX₃, NO₂, OH, and alkoxy;
R² is selected from the group consisting of H, X, CX₃, NO₂, OH, and alkoxy;
R³ is selected from the group consisting of X, CX₃, NO₂, OH, and alkoxy; and
R⁴ is selected from the group consisting of H and C(O)R⁵,
wherein R⁵ is selected from the group consisting of H, optionally substituted C₁₋₁₈ alkyl, optionally substituted C₂₋₁₈ alkenyl, and optionally substituted C₂₋₁₈ alkynyl; and
wherein each X is an independently selected halogen.
20. The method of embodiment 19, wherein R¹ is CX₃ or NO₂.
21. The method of embodiment 19 or 20, wherein R² is H or X.
22. The method of any one of embodiments 19 to 21, wherein R³ is X.
23. The method of any one of embodiments 19 to 22, wherein R⁵ is C₂ alkyl or C₂ alkenyl.
24. The method of any one of embodiments 19 to 23, wherein X is independently selected from the group consisting of F and Cl.
25. The method of any one of embodiments 19 to 24, wherein the compound of Formula (I) is selected from the group consisting of

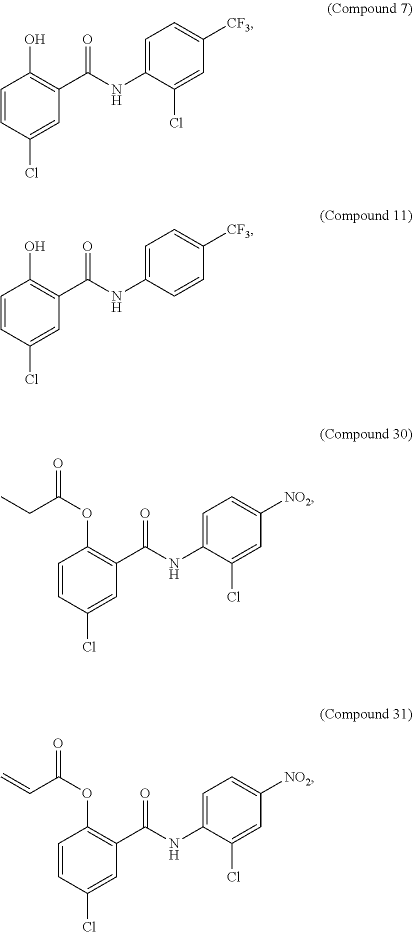 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.
26. The method of any one of embodiments 19 to 25, wherein the antiandrogen drug is selected from the group consisting of a non-steroidal androgen receptor antagonist, a CYP17A1 inhibitor, and a combination thereof.
27. The composition of embodiment 26, wherein the antiandrogen drug is selected from the group consisting of bicalutamide, apalutamide, enzalutamide, abiraterone acetate, and a combination thereof.
28. The method of any one of embodiments 19 to 27, wherein the expression and/or activity of an androgen receptor or a variant thereof is inhibited.
29. The method of embodiment 28, wherein the androgen receptor variant is selected from the group consisting of a splice variant, a mutant variant, and a combination thereof.
30. The method of embodiment 29, wherein the splice variant is an AR-V1, AR-V3, AR-V7, AR-V9, and/or AR-V12 splice variant.
31. The method of embodiment 30, wherein the splice variant is an AR-V7 splice variant.
32. The method of embodiment 29, wherein the mutant variant comprises one or more mutations selected from the group consisting of K581R, L702H, T878A, V716M, and a combination thereof relative to the amino acid sequence set forth in SEQ ID NO: 1.
33. The method of any one of embodiments 19 to 32, wherein the cancer is prostate cancer or breast cancer.
34. The method of any one of embodiments 19 to 33, wherein the cancer is selected from the group consisting of an androgen-independent cancer, a metastatic cancer, a castrate-resistant cancer, a castration recurrent cancer, a hormone-resistant cancer, a metastatic castrate-resistant cancer, and a combination thereof.
35. The method of embodiment 34, wherein the androgen independence, castrate resistance, or hormone resistance of the cancer is decreased or reversed.
36. The method of any one of embodiments 19 to 35, wherein the composition further comprises a pharmaceutically acceptable carrier.
37. The method of any one of embodiments 19 to 36, wherein the antiandrogen drug and the compound of Formula (I) are given concomitantly.
38. The method of any one of embodiments 19 to 36, wherein the antiandrogen drug and the compound of Formula (I) are given sequentially.
39. The method of any one of embodiments 19 to 38, wherein the subject does not have cancer.
40. The method of any one of embodiments 19 to 38, wherein treating the subject results in an improvement in one or more symptoms of the cancer.
41. The method of any one of embodiments 19 to 40, wherein a test sample is obtained from the subject before and/or after the antiandrogen drug and the compound of Formula (I) are administered to the subject.
42. The method of embodiment 41, wherein the test sample comprises tissue, blood, or a combination thereof.
43. The method of embodiment 42, wherein the test tissue sample comprises cancer tissue.
44. The method of any one of embodiments 41 to 43, wherein the level of one or more biomarkers is determined in the test sample.
45. The method of embodiment 44, wherein the one or more biomarkers comprises prostate-specific antigen (PSA).
46. The method of embodiment 44 or 45, wherein the level of the one or more biomarkers in the test sample is compared to the level of the one or more biomarkers in a reference sample.
47. The method of embodiment 46, wherein the reference sample is normal blood or tissue obtained from the same subject before and/or after the antiandrogen drug and the compound of Formula (I) are administered to the subject.
48. The method of embodiment 46, wherein the reference sample is obtained from a different subject or a population of subjects.
49. The method of embodiment 46 or 48, wherein the level of PSA in the test sample is higher than the level of PSA in the reference sample, and wherein the test sample is obtained before the antiandrogen drug and the compound of Formula (I) are administered to the subject.
50. The method of any one of embodiments 45 to 49, wherein administering the antiandrogen drug and the compound of Formula (I) to the subject results in a decrease in the level of PSA in a test sample obtained from the subject after administration compared to a test sample obtained from the subject before administration.
51. A method for inhibiting the expression and/or activity of an androgen receptor in a cell, the method comprising contacting the androgen receptor or the cell with a therapeutically effective amount of a composition comprising an antiandrogen drug and a compound according to Formula (I):

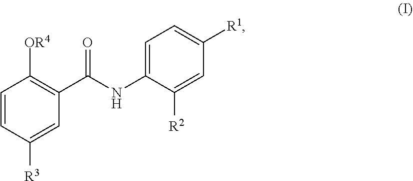

wherein:
R¹ is selected from the group consisting of X, CX₃, NO₂, OH, and alkoxy;
R² is selected from the group consisting of H, X, CX₃, NO₂, OH, and alkoxy;
R³ is selected from the group consisting of X, CX₃, NO₂, OH, and alkoxy; and
R⁴ is selected from the group consisting of H and C(O)R⁵,
wherein R⁵ is selected from the group consisting of H, optionally substituted C₁₋₁₈ alkyl, optionally substituted C₂₋₁₈ alkenyl, and optionally substituted C₂₋₁₈ alkynyl; and
wherein each X is an independently selected halogen.
52. The method of embodiment 51, wherein R¹ is CX₃ or NO₂.
53. The method of embodiment 51 or 52, wherein R² is H or X.
54. The method of any one of embodiments 51 to 53, wherein R³ is X.
55. The method of any one of embodiments 51 to 54, wherein R⁵ is C₂ alkyl or C₂ alkenyl.
56. The method of any one of embodiments 51 to 55, wherein X is independently selected from the group consisting of F and Cl.
57. The method of any one of embodiments 51 to 56, wherein the compound of Formula (I) is selected from the group consisting of

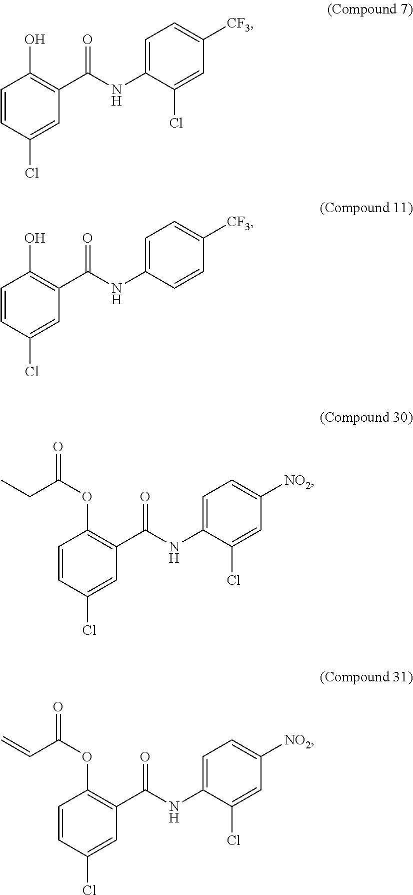 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.
58. The method of any one of embodiments 51 to 57, wherein the antiandrogen drug is selected from the group consisting of a non-steroidal androgen receptor antagonist, a CYP17A1 inhibitor, and a combination thereof.
59. The method of embodiment 58, wherein the antiandrogen drug is selected from the group consisting of bicalutamide, apalutamide, enzalutamide, abiraterone acetate, and a combination thereof.
60. The method of any one of embodiments 51 to 59, wherein androgen receptor transactivation is inhibited.
61. The method of any one of embodiments 51 to 60, wherein androgen receptor expression is inhibited.
62. The method of any one of embodiments 51 to 61, wherein androgen receptor-mediated transcriptional activity is inhibited.
63. The method of any one of embodiments 51 to 62, wherein the expression and/or activity of an androgen receptor variant is inhibited.
64. The method of embodiment 63, wherein recruitment of the androgen receptor variant to a prostate-specific antigen (PSA) promoter is inhibited.
65. The method of embodiment 63 or 64, wherein the androgen receptor variant is selected from the group consisting of a splice variant, a mutant variant, and a combination thereof.
66. The method of embodiment 65, wherein the splice variant is an AR-V1, AR-V3, AR-V7, AR-V9, and/or AR-V12 splice variant.
67. The method of embodiment 66, wherein the splice variant is an AR-V7 splice variant.
68. The method of embodiment 65, wherein the mutant variant comprises one or more mutations selected from the group consisting of K581R, L702H, T878A, V716M, and a combination thereof relative to the amino acid sequence set forth in SEQ ID NO: 1.
69. The method of any one of embodiments 51 to 68, wherein the cell is a cancer cell.
70. The method of embodiment 69, wherein the cancer cell is a metastatic cancer cell.
71. The method of embodiment 69 or 70, wherein the cancer cell is a prostate cancer cell or a breast cancer cell.
72. The method of any one of embodiments 69 to 71, wherein the cancer cell is selected from the group consisting of an androgen-independent cancer cell, a castrate-resistant cancer cell, a hormone-resistant cancer cell, and a combination thereof.
73. The method of embodiment 72, wherein the androgen independence, castrate resistance, and/or hormone resistance of the cancer cell is reduced, decreased, or reversed.
74. The method of any one of embodiments 69 to 73, wherein the cancer cell is resensitized to the antiandrogen drug.
75. The method of any one of embodiments 69 to 73, wherein resistance of the cancer cell to the antiandrogen drug is reduced, decreased, or reversed.
76. The method of any one of embodiments 69 to 75, wherein the invasive ability of the cancer cell and/or the ability of the cancer cell to migrate is inhibited.
77. The method of any one of embodiments 69 to 76, wherein the ability of the cancer cell to grow and/or form a colony is inhibited.
78. The method of any one of embodiments 51 to 77, wherein the composition further comprises a pharmaceutically acceptable carrier.
79. A kit for preventing or treating cancer in a subject comprising an antiandrogen drug and a compound according to Formula (I):
wherein:

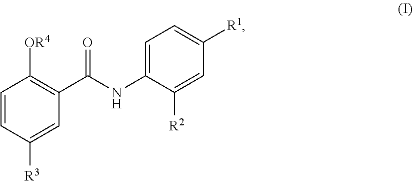

R¹ is selected from the group consisting of X, CX₃, NO₂, OH, and alkoxy;
R² is selected from the group consisting of H, X, CX₃, NO₂, OH, and alkoxy;
R³ is selected from the group consisting of X, CX₃, NO₂, OH, and alkoxy; and
R⁴ is selected from the group consisting of H and C(O)R⁵,
wherein R⁵ is selected from the group consisting of H, optionally substituted C₁₋₁₈ alkyl, optionally substituted C₂₋₁₈ alkenyl, and optionally substituted C₂₋₁₈ alkynyl; and
wherein each X is an independently selected halogen.
80. The kit of embodiment 79, wherein R¹ is CX₃ or NO₂.
81. The kit of embodiment 79 or 80, wherein R² is H or X.
82. The kit of any one of embodiments 79 to 81, wherein R³ is X.
83. The kit of any one of embodiments 79 to 82, wherein R⁵ is C₂ alkyl or C₂ alkenyl.
84. The kit of any one of embodiments 79 to 83, wherein X is independently selected from the group consisting of F and Cl.
85. The kit of any one of embodiments 79 to 84, wherein the compound of Formula (I) is selected from the group consisting of

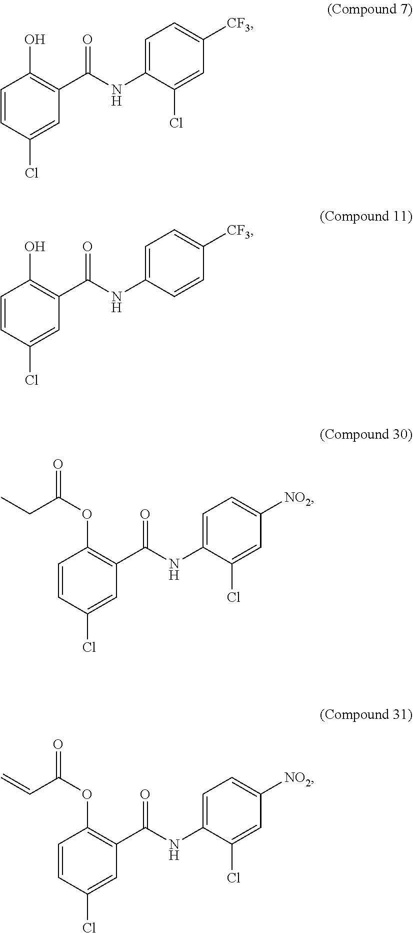 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]

and a combination thereof.
86. The kit of any one of embodiments 79 to 85, wherein the antiandrogen drug is selected from the group consisting of a non-steroidal androgen receptor antagonist, a CYP17A1 inhibitor, and a combination thereof.
87. The kit of embodiment 86, wherein the antiandrogen drug is selected from the group consisting of bicalutamide, apalutamide, enzalutamide, abiraterone acetate, and a combination thereof.
88. The kit of any one of embodiments 79 to 87, further comprising a pharmaceutically acceptable carrier.
89. The kit of any one of embodiments 79 to 88, wherein the cancer is prostate cancer or breast cancer.
90. The kit of any one of embodiments 79 to 89, wherein the cancer is selected from the group consisting of an androgen-independent cancer, a metastatic cancer, a castrate-resistant cancer, a castration recurrent cancer, a hormone-resistant cancer, a metastatic castrate-resistant cancer, and a combination thereof.
91. The kit of any one of embodiments 79 to 90, further comprising instructions for use.
92. The kit of any one of embodiments 79 to 91, further comprising paraphernalia and/or one or more reagents for administering the antiandrogen drug and/or the compound of Formula (I) to the subject.
93. The kit of any one of embodiments 79 to 92, further comprising paraphernalia and/or one or more reagents for obtaining a sample from the subject.
94. The kit of embodiment 93, further comprising paraphernalia and/or one or more reagents for determining the level of one or more biomarkers in the sample.
95. The kit of embodiment 94, wherein the one or more biomarkers comprises prostate-specific antigen (PSA).
96. The kit of any one of embodiments 79 to 95, further comprising negative and/or positive control samples.
97. A composition comprising an antiandrogen drug and a compound according to Formula (II):
wherein:

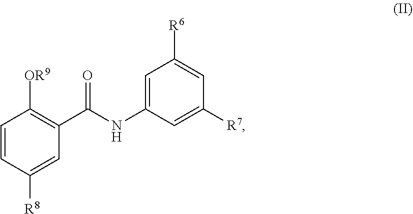

R⁶ and R⁷ are independently selected from the group consisting of H, X, CX₃, NO₂, OH, and alkoxy;
R⁸ is selected from the group consisting of X, CX₃, NO₂, OH, and alkoxy; and
R⁹ is selected from the group consisting of H and C(O)R^{th},
wherein R¹⁹ is selected from the group consisting of H, optionally substituted C₁₋₁₈ alkyl, optionally substituted C₂₋₁₈ alkenyl, and optionally substituted C₂₋₁₈ alkynyl; and wherein each X is an independently selected halogen.
98. The composition of embodiment 97, wherein R⁶ and/or R⁷ are CX₃.
99. The composition of embodiment 97 or 98, wherein R⁸ is X.
100. The composition of any one of embodiments 97 to 99, wherein R⁹ is H.
101. The composition of any one of embodiments 97 to 100, wherein X is independently selected from the group consisting of F and Cl.
102. The composition of any one of embodiments 97 to 101, wherein the compound of Formula (II) is

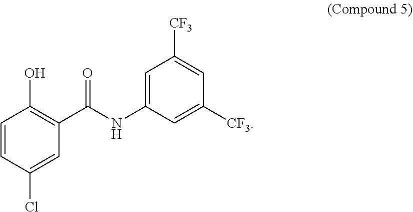 [SMILES: O=C(Nc1cc(C(F)(F)F)cc(C(F)(F)F)c1)c1cc(Cl)ccc1O]

103. The composition of any one of embodiments 97 to 102, wherein the antiandrogen drug is selected from the group consisting of a non-steroidal androgen receptor antagonist, a CYP17A1 inhibitor, and a combination thereof.
104. The composition of embodiment 103, wherein the antiandrogen drug is selected from the group consisting of bicalutamide, apalutamide, enzalutamide, abiraterone acetate, and a combination thereof.
105. The composition of any one of embodiments 97 to 104, wherein the composition inhibits the expression and/or activity of an androgen receptor or a variant thereof.
106. The composition of embodiment 105, wherein the androgen receptor variant is selected from the group consisting of a splice variant, a mutant variant, and a combination thereof.
107. The composition of embodiment 106, wherein the splice variant is an AR-V1, AR-V3, AR-V7, AR-V9, and/or AR-V12 splice variant.
108. The composition of embodiment 107, wherein the splice variant is an AR-V7 splice variant.
109. The composition of embodiment 106, wherein the mutant variant comprises one or more mutations selected from the group consisting of K581R, L702H, T878A, V716M, and a combination thereof relative to the amino acid sequence set forth in SEQ ID NO: 1.
110. The composition of any one of embodiments 97 to 109, wherein the composition is an effective inhibitor of cancer cell proliferation.
111. The composition of embodiment 110, wherein the cancer cell is a prostate cancer cell or a breast cancer cell.
112. The composition of embodiment 110 or 111, wherein the cancer cell is selected from the group consisting of an androgen-independent cancer cell, a metastatic cancer cell, a castrate-resistant cancer cell, a castration recurrent cancer cell, a hormone-resistant cancer cell, a metastatic castrate-resistant cancer cell, and a combination thereof.
113. The composition of any one of embodiments 97 to 112, further comprising a pharmaceutically acceptable carrier.
114. A method for preventing or treating cancer in a subject, the method comprising administering to the subject a therapeutically effective amount of the composition of any one of embodiments 97 to 113.
115. A method for inhibiting the expression and/or activity of an androgen receptor in a cell, the method comprising contacting the androgen receptor or the cell with a therapeutically effective amount of the composition of any one of embodiments 97 to 113.
116. A kit for preventing or treating cancer in a subject comprising the composition of any one of embodiments 97 to 113.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

Informal Sequence Listing

| SEQ | | Des- |
|---|---|---|
| ID NO: | Sequence | cription |
| 1 | MEVQLGLGRVYPRPPSKTYRGAFQNLFQSVREVIQNPGPRHP | Human |
| | EAASAAPPGASLLLLQQQQQQQQQQQQQQQQQQQQQQQET | androgen |
| | SPRQQQQQQGEDGSPQAHRRGPTGYLVLDEEQQPSQPQSAL | receptor |
| | ECHPERGCVPEPGAAVAASKGLPQQLPAPPDEDDSAAPSTLS | isoform 1 |
| | LLGPTFPGLSSCSADLKDILSEASTMQLLQQQQQEAVSEGSSS | |
| | GRAREASGAPTSSKDNYLGGTSTISDNAKELCKAVSVSMGL | |
| | GVEALEHLSPGEQLRGDCMYAPLLGVPPAVRPTPCAPLAEC | |
| | KGSLLDDSAGKSTEDTAEYSPFKGGYTKGLEGESLGCSGSAA | |
| | AGSSGTLELPSTLSLYKSGALDEAAAYQSRDYYNFPLALAGP | |
| | PPPPPPPHPHARIKLENPLDYGSAWAAAAAQCRYGDLASLH | |
| | GAGAAGPGSGSPSAAASSSWHTLFTAEEGQLYGPCGGGGGG | |
| | GGGGGGGGGGGGGGGGGEAGAVAPYGYTRPPQGLAGQES | |
| | DFTAPDVWYPGGMVSRVPYPSPTCVKSEMGPWMDSYSGPY | |
| | GDMRLETARDHVLPIDYYFPPQKTCLICGDEASGCHYGALTC | |
| | GSCKVFFKRAAEGKQKYLCASRNDCTIDKFRRKNCPSCRLR | |
| | KCYEAGMTLGARKLKKLGNLKLQEEGEASSTTSPTEETTQK | |
| | LTVSHIEGYECQPIFLNVLEAIEPGVVCAGHDNNQPDSFAALL | |
| | SSLNELGERQLVHVVKWAKALPGFRNLHVDDQMAVIQYSW | |
| | MGLMVFAMGWRSFTNVNSRMLYFAPDLVFNEYRMHKSRM | |
| | YSQCVRMRHLSQEFGWLQITPQEFLCMKALLLFSIIPVDGLK | |
| | NQKFFDELRMNYIKELDRIIACKRKNPTSCSRRFYQLTKLLDS | |
| | VQPIARELHQFTFDLLIKSHMVSVDFPEMMAEIISVQVPKILS | |
| | GKVKPIYFHTQ | |
| 2 | MEVQLGLGRVYPRPPSKTYRGAFQNLFQSVREVIQNPGPRHP | AR-V9 |
| | EAASAAPPGASLLLQQQQQQQQQQQQQQQQQQQQQQQQQ | sequence |
| | QQQQETSPRQQQQQQGEDGSPQAHRRGPTGYLVLDEEQQPS | |
| | QPQSALECHPERGCVPEPGAAVAASKGLPQQLPAPPDEDDSA | |
| | APSTLSLLGPTFPGLSSCSADLKDILSEASTMQLLQQQQQEAV | |
| | SEGSSSGRAREASGAPTSSKDNYLGGTSTISDNAKELCKAVS | |
| | VSMGLGVEALEHLSPGEQLRGDCMYAPLLGVPPAVRPTPCA | |
| | PLAECKGSLLDDSAGKSTEDTAEYSPFKGGYTKGLEGESLGC | |
| | SGSAAAGSSGTLELPSTLSLYKSGALDEAAAYQSRDYYNFPL | |
| | ALAGPPPPPPPPHPHARIKLENPLDYGSAWAAAAAQCRYGD | |
| | LASLHGAGAAGPGSGSPSAAASSSWHTLFTAEEGQLYGPCG | |
| | GGGGGGGGGGGGGGGGGGEAGAVAPYGYTRPPQGLAGQE | |
| | SDFTAPDVWYPGGMVSRVPYPSPTCVKSEMGPWMDSYSGP | |
| | YGDMRLETARDHVLPIDYYFPPQKTCLICGDEASGCHYGAL | |
| | TCGSCKVFFKRAAEGKQKYLCASRNDCTIDKFRRKNCPSCR | |
| | LRKCYEAGMTLGDNLPEQAAFWRHLHIFWDHVVKK | |

1. A composition comprising an antiandrogen drug and a compound according to Formula (I): 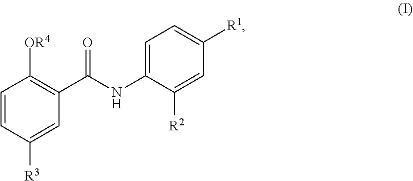
wherein:
R¹ is selected from the group consisting of X, CX₃, NO₂, OH, and alkoxy;
R² is selected from the group consisting of H, X, CX₃, NO₂, OH, and alkoxy;
R³ is selected from the group consisting of X, CX₃, NO₂, OH, and alkoxy; and
R⁴ is selected from the group consisting of H and C(O)R⁵,
wherein R⁵ is selected from the group consisting of H, optionally substituted C₁₋₁₈ alkyl, optionally substituted C₂₋₁₈ alkenyl, and optionally substituted C₂₋₁₈ alkynyl; and
wherein each X is an independently selected halogen.

2. The composition of claim 1, wherein le is CX₃ or NO₂.

3. The composition of claim 1, wherein R² is H or X.

4. The composition of claim 1, wherein R³ is X.

5. The composition of claim 1, wherein R⁵ is C₂ alkyl or C₂ alkenyl.

6. The composition of claim 1, wherein X is independently selected from the group consisting of F and Cl.

7. The composition of claim 1, wherein the compound of Formula (I) is selected from the group consisting of 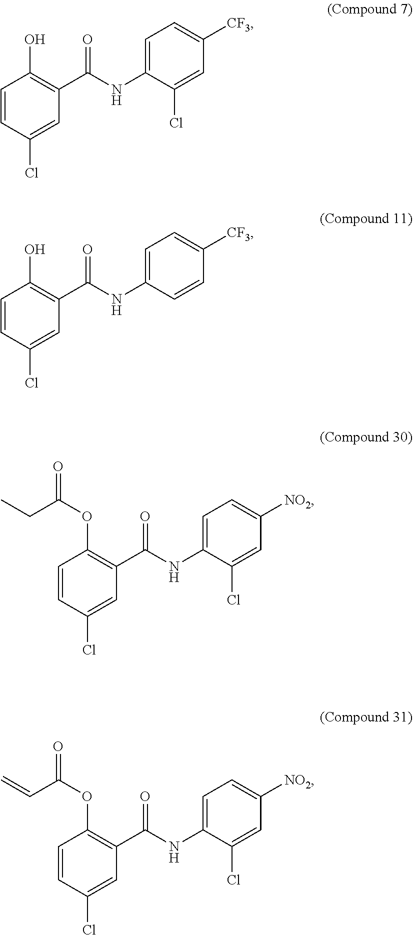 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]
and a combination thereof.

8. The composition claim 1, wherein the antiandrogen drug is selected from the group consisting of a non-steroidal androgen receptor antagonist, a CYP17A1 inhibitor, and a combination thereof.

9. The composition of claim 8, wherein the antiandrogen drug is selected from the group consisting of bicalutamide, apalutamide, enzalutamide, abiraterone acetate, and a combination thereof.

10. The composition of claim 1, wherein the composition inhibits the expression and/or activity of an androgen receptor or a variant thereof.

11-12. (canceled)

13. The composition of claim 1, further comprising a pharmaceutically acceptable carrier.

14. A method for preventing or treating cancer in a subject, the method comprising administering to the subject a therapeutically effective amount of an antiandrogen drug and a compound according to Formula (I): 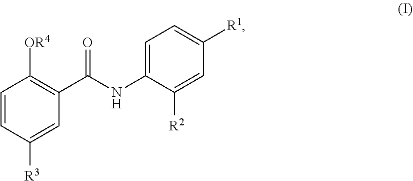
wherein:
R¹ is selected from the group consisting of X, CX₃, NO₂, OH, and alkoxy;
R² is selected from the group consisting of H, X, CX₃, NO₂, OH, and alkoxy;
R³ is selected from the group consisting of X, CX₃, NO₂, OH, and alkoxy; and
R⁴ is selected from the group consisting of H and C(O)R⁵,
wherein R⁵ is selected from the group consisting of H, optionally substituted C₁₋₁₈ alkyl, optionally substituted C₂₋₁₈ alkenyl, and optionally substituted C₂₋₁₈ alkynyl; and
wherein each X is an independently selected halogen.

15. The method of claim 14, wherein R¹ is CX₃ or NO₂.

16. The method of claim 14, wherein R² is H or X.

17. The method of claim 14, wherein R³ is X.

18. The method of claim 14, wherein R⁵ is C₂ alkyl or C₂ alkenyl.

19. The method of claim 14, wherein X is independently selected from the group consisting of F and Cl.

20. The method of claim 14, wherein the compound of Formula (I) is selected from the group consisting of 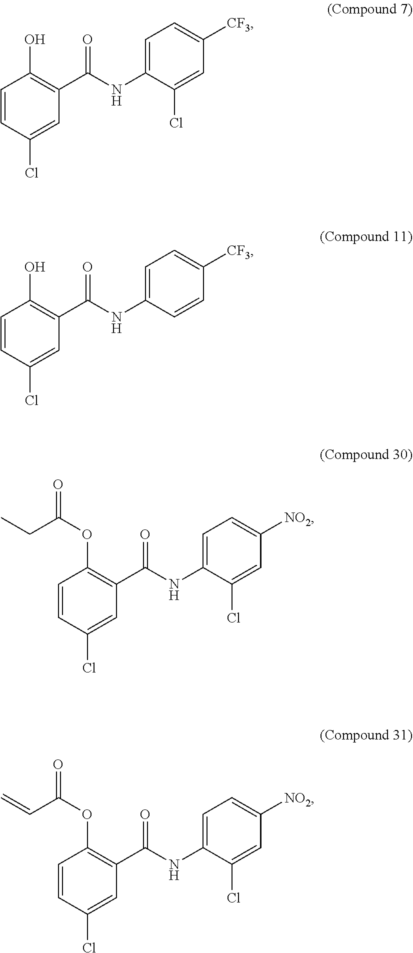 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]
and a combination thereof.

21. The method of claim 14, wherein the antiandrogen drug is selected from the group consisting of a non-steroidal androgen receptor antagonist, a CYP17A1 inhibitor, and a combination thereof.

22. The composition of claim 21, wherein the antiandrogen drug is selected from the group consisting of bicalutamide, apalutamide, enzalutamide, abiraterone acetate, and a combination thereof.

23. The method of claim 14, wherein the expression and/or activity of an androgen receptor or a variant thereof is inhibited.

24-25. (canceled)

26. The method of claim 14, wherein the cancer is prostate cancer or breast cancer.

27. (canceled)

28. (canceled)

29. The method of claim 14, wherein the antiandrogen drug and the compound according to Formula (I) are in a composition that further comprises a pharmaceutically acceptable carrier.

30. The method of claim 14, wherein the antiandrogen drug and the compound of Formula (I) are given concomitantly.

31. The method of claim 14, wherein the antiandrogen drug and the compound of Formula (I) are given sequentially.

32-33. (canceled)

34. A method for inhibiting the expression and/or activity of an androgen receptor in a cell, the method comprising contacting the androgen receptor or the cell with a therapeutically effective amount of an antiandrogen drug and a compound according to Formula (I): 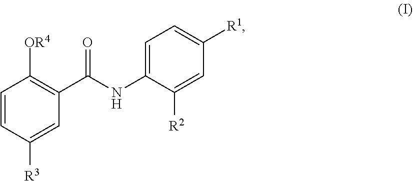
wherein:
R¹ is selected from the group consisting of X, CX₃, NO₂, OH, and alkoxy;
R² is selected from the group consisting of H, X, CX₃, NO₂, OH, and alkoxy;
R³ is selected from the group consisting of X, CX₃, NO₂, OH, and alkoxy; and
R⁴ is selected from the group consisting of H and C(O)R⁵,
wherein R⁵ is selected from the group consisting of H, optionally substituted C₁₋₁₈ alkyl, optionally substituted C₂₋₁₈ alkenyl, and optionally substituted C₂₋₁₈ alkynyl; and
wherein each X is an independently selected halogen.

35. The method of claim 34, wherein R¹ is CX₃ or NO₂.

36. The method of claim 34, wherein R² is H or X.

37. The method of claim 34, wherein R³ is X.

38. The method of claim 34, wherein R⁵ is C₂ alkyl or C₂ alkenyl.

39. The method of claim 34, wherein X is independently selected from the group consisting of F and Cl.

40. The method of claim 34, wherein the compound of Formula (I) is selected from the group consisting of 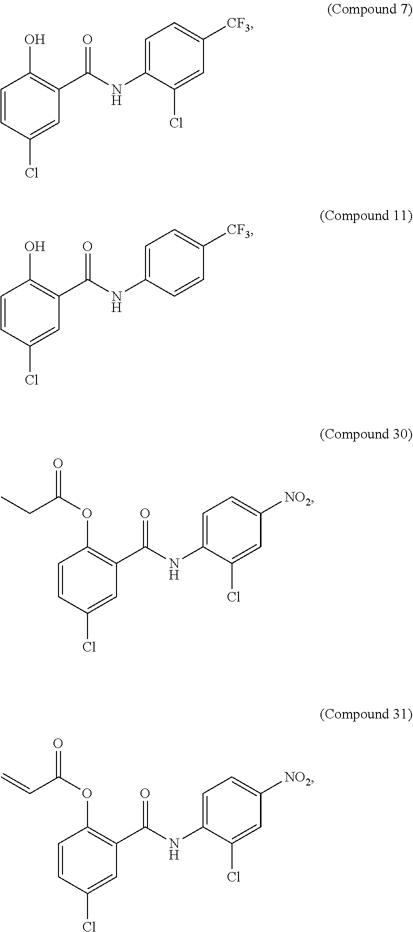 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]
and a combination thereof.

41. The method of claim 34, wherein the antiandrogen drug is selected from the group consisting of a non-steroidal androgen receptor antagonist, a CYP17A1 inhibitor, and a combination thereof.

42. The method of claim 41, wherein the antiandrogen drug is selected from the group consisting of bicalutamide, apalutamide, enzalutamide, abiraterone acetate, and a combination thereof.

43. The method of claim 34, wherein androgen receptor transactivation, androgen receptor expression, androgen receptor-mediated transcriptional activity, or the expression and/or activity of an androgen receptor variant is inhibited.

61-77. (canceled)

78. The method of claim 34, wherein the antiandrogen drug and the compound according to Formula (I) are in a composition that further comprises a pharmaceutically acceptable carrier.

79. A kit for preventing or treating cancer in a subject comprising an antiandrogen drug and a compound according to Formula (I): 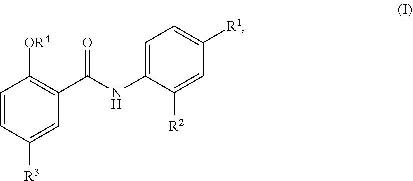
wherein:
R¹ is selected from the group consisting of X, CX₃, NO₂, OH, and alkoxy;
R² is selected from the group consisting of H, X, CX₃, NO₂, OH, and alkoxy;
R³ is selected from the group consisting of X, CX₃, NO₂, OH, and alkoxy; and
R⁴ is selected from the group consisting of H and C(O)R⁵,
wherein R⁵ is selected from the group consisting of H, optionally substituted C₁₋₁₈ alkyl, optionally substituted C₂₋₁₈ alkenyl, and optionally substituted C₂₋₁₈ alkynyl; and
wherein each X is an independently selected halogen.

80. The kit of claim 79, wherein R¹ is CX₃ or NO₂.

81. The kit of claim 79, wherein R² is H or X.

82. The kit of claim 79, wherein R³ is X.

83. The kit of claim 79, wherein R⁵ is C₂ alkyl or C₂ alkenyl.

84. The kit of claim 79, wherein X is independently selected from the group consisting of F and Cl.

85. The kit of claim 79, wherein the compound of Formula (I) is selected from the group consisting of 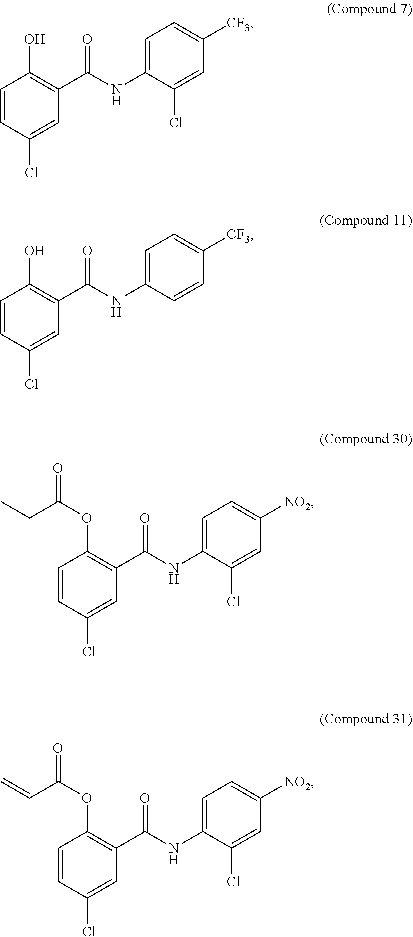 [SMILES: O=C(Nc1ccc(C(F)(F)F)cc1Cl)c1cc(Cl)ccc1O ; O=C(Nc1ccc(C(F)(F)F)cc1)c1cc(Cl)ccc1O ; CCC(=O)Oc1ccc(Cl)cc1C(=O)Nc1ccc([N+](=O)[O-])cc1Cl ; C=CC(=O)Oc1ccc(Cl)cc1C(=O)Nc1ccc([N+](=O)[O-])cc1Cl]
and a combination thereof.

86. The kit of claim 79, wherein the antiandrogen drug is selected from the group consisting of a non-steroidal androgen receptor antagonist, a CYP17A1 inhibitor, and a combination thereof.

87. The kit of claim 86, wherein the antiandrogen drug is selected from the group consisting of bicalutamide, apalutamide, enzalutamide, abiraterone acetate, and a combination thereof.

88. The kit of claim 79, further comprising a pharmaceutically acceptable carrier.

89. The kit of claim 79, wherein the cancer is prostate cancer or breast cancer.

90-116. (canceled)


SEQUENCE LISTING (2 sequences)

SEQ ID NO: 1 - protein, 920 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 2 - protein, 652 aa, Unknown
  androgen receptor variant; genus unknown
MEVQLGLGRVYPRPPSKTYRGAFQNLFQSVREVIQNPGPRHPEAASAAPPGASLLLQQQQQQQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGDNLPEQAAFWRHLHIFWDHVVKK